(19) United States
(12) Patent Application Publication
(10) Pub. No.: US 2014/0148378 A1
(43) Pub. Date: May 29, 2014

(54) ANTIBACTERIAL PEPTIDES

(71) Applicant: THE REGENTS OF THE UNIVERSITY OF CALIFORNIA, (US)

(72) Inventors: Carlito B. LEBRILLA, Davis, CA (US); Andrés GUERRERO, Davis, CA (US); David C. DALLAS, Davis, CA (US); J. Bruce GERMAN, Davis, CA (US); Nora KHALDI, Dublin (IE)

(73) Assignee: THE REGENTS OF THE UNIVERSITY OF CALIFORNIA, Oakland, CA (US)

(21) Appl. No.: 13/781,760

(22) Filed: Mar. 1, 2013

Related U.S. Application Data
(60) Provisional application No. 61/730,302, filed on Nov. 27, 2012.

(51) Int. Cl. C07K 14/00; C07K 7/08; C07K 7/06

(52) U.S. Cl. CPC C07K 14/00; C07K 7/06; C07K 7/08
USPC 514/2.4; 530/330; 530/324; 530/325; 530/326; 530/327; 530/328; 530/329

(57) ABSTRACT

The present invention relates to antibacterial peptides and analogs thereof, e.g., originating from, derived from, isolated and/or purified from mammalian milk, that reduce, inhibit and/or prevent the growth or proliferation of a bacterial organism.

### CROSS-REFERENCE TO RELATED APPLICATIONS

This application claims the benefit under 35 U.S.C. §119(e) of U.S. Provisional Application No. 61/730,302, filed on Nov. 27, 2012, which is hereby incorporated herein by reference in its entirety for all purposes.

### FIELD

The present invention relates to antibacterial peptides and analogs thereof, e.g., originating from, derived from, isolated and/or purified from mammalian milk, that reduce, inhibit and/or prevent the growth or proliferation of a bacterial organism.

### BACKGROUND

Human milk contains active proteases, namely plasmin (Warner, et al., J Am Chem Soc (1945) 67(4):529-532; Okamoto, et al., Thromb Haemostasis (1981) 45(2):121; Korycha-Dahl, et al., J Dairy Sci (1983) 66(4):704-711; Astrup and Sterndorff, in “A Fibrinolytic System in Human Milk,” Royal Society of Medicine: (1953) 605-608), trypsin (Borulf, et al., Acta Paediatrica (1987) 76(1):11-15), elastase (Borulf, et al., supra), cathepsin D (V\check{e}tvicka, et al., Biochemistry and Molecular Biology International (1993) 30(5):921) and kallikrein (Palmer, et al., Proteomics (2006) 6(7):2208-2216). However, anti-proteases, namely, α-1-antitryspin and α1-antichymotrypsin, are also present in milk (Lindberg, et al., Pediatr. Res. (1982) 16(6):479-483; Lindberg, Pediatr. Res. (1979) 13(9):969-972; McGilligan, et al., Pediatr. Res. (1987) 22(3):268-270). The presence of proteases and anti-proteases in breast milk suggests that a balance of proteolytic degradation in the mammary gland is important for the infant's health (Dallas, et al., J Nutr Disorders Ther (2012) 2(112): 2161-0509.1000112).

Mother's milk evolved over more than 200 million years to nourish and protect the neonate (Oftedal, Journal of Mammary Gland Biology and Neoplasia (2002) 7(3):225-252). A large number of milk peptides produced by in vitro digestion have been found to be bioactive (Dallas, et al., supra). Bioactivities of milk peptides include immunomodulation (Migliore-Samour, et al., J. Dairy Res. (1989) 56(3):357-362; Jorgensen, et al., Journal of Peptide Science (2010) 16(1):21-30), opioid-like activity (Kampa, et al., Biochem J (1996) 319,(Pt 3):903; Brantl, et al., Eur. J. Pharmacol. (1984) 106(1):213-214), antimicrobial action (Liepke, et al., Journal of Chromatography (2001) 752(2):369-377; Aniansson, et al., Microb. Pathog. (1990) 8(5): 315-323; Stromqvist, et al., J Pediatr Gastr Nutr (1995) 21(3):288-296) and probiotic action (Liepke, et al., Eur. J. Biochem. (2002) 269(2):712-718; Bezkorovainy, et al., Am Soc Nutrition (1979) 32:1428-1432; Azuma, et al., Agricultural and Biological Chemistry (1984) 48(8):2159-2162). These peptide fragments often exist within milk proteins that, when intact, are not bioactive (Schanbacher, et al., Livestock Production Science (1997) 50(1-2):105-123). Specific proteolysis releases these encrypted bioactive fragments. Some proteolytic events heighten functions of intact milk proteins; for example, digestion of human lactoferrin by gastric pepsin produces the peptide fragment lactoferricin that has 10-100 times stronger bactericidal effects than the parent protein (Bellamy, et al., Biochim Biophys Acta (1992) 1121(1-2):130-136).

Different approaches to identify naturally-occurring peptides in human milk have been tested. Ferranti et al. found—via matrix-assisted laser desorption ionization (MALDI) and electrospray mass spectrometry (ESI-MS)—93 β-casein peptides, 4 asl-casein peptides and 13 κ-casein peptides in milk from mothers giving birth either preterm or at term (Ferranti, et al., J. Dairy Res. (2004) 71(1):74-87). The cleavage positions of the peptides found in that paper suggested that plasmin is the main enzyme involved in the hydrolysis of proteins of human milk. Armaforte et al. confirmed the presence of low-molecular weight fragments (but not the exact sequences) of β- and αs1-casein in human milk by 2D-SDS-PAGE followed by trypsin digestion of gel spots and mass spectrometry (Armaforte, et al., Int Dairy J (2010) 20(10):715-723). Christensen et al. found seven naturally-occurring peptide fragments of osteopontin, another common milk protein, in intact human milk via immunoaffinity extraction and mass spectrometry (Christensen, et al., Journal of Biological Chemistry (2010) 285(11):7929-37). However, all these studies are focused on the hydrolytic products of specific milk proteins and lack a complete description of the complete protein-released peptidome.

The lactating mammary gland is at constant risk of mastitis in part due to the conditions of the mammary gland and immune system of a lactating mother. This inflammatory syndrome is destructive and can result in blocked milk ducts, abscesses and septicemia and accounts for approximately 25% of women's decisions to wean their infants.

### SUMMARY

In one aspect, a composition comprising or consisting essentially of one or more peptides isolated and/or purified from mammalian milk is provided; the peptides in the composition reduce, inhibit and/or prevent the growth or proliferation of a bacterial organism. In some embodiments, the isolated and/or purified peptides have a molecular weight in the range of about 0.4 kDa to about 5.8 kDa, e.g., about 0.5-5.0 kDa, about 0.6-4.5 kDa, about 0.7-4.0 kDa, about 0.8-3.5 kDa, e.g., have a molecular weight that is at least about 0.4 kDa, 0.5 kDa, 0.6 kDa, 0.7 kDa, 0.8 kDa and up to about 3.5 kDa, 4.0 kDa, 4.5 kDa, 5.0 kDa, 5.5 kDa or about 5.8 kDa. In varying embodiments, the peptides have from about 5 to about 55 amino acid residues, e.g., from about 6 to about 50 amino acid residues, from about 7 to about 45 amino acid residues, from about 8 to about 40 amino acid residues, from about 9 to about 35 amino acid residues or from about 10 to about 20 residues, e.g., about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50, 51, 52, 53, 54, 55 amino acid residues. In varying embodiments, the composition does not comprise non-protein and/or non-peptide components from mammalian milk.

In a further aspect, isolated and/or purified peptides that reduce, inhibit and/or prevent the growth or proliferation of a bacterial organism are provided. In some embodiments, an antibacterial peptide comprising from 5 to 55 amino acid residues, e.g., from about 6 to about 50 amino acid residues, from about 7 to about 45 amino acid residues, from about 8 to about 40 amino acid residues, from about 9 to about 35 amino acid residues is provided. In some embodiments, the peptide comprises or consists essentially of a subsequence of a protein selected from the group consisting of: polymeric immunoglobulin receptor (PIGR); beta-casein (CASB); alpha-S1-casein (CASA1); butyrophilin subfamily 1 member A1 (BT1A1); osteopontin (OSTP); mucin-1 (MUC1); perilipin-2 (PLIN2); neural Wiskott-Aldrich syndrome protein (WASL); cancer susceptibility candidate gene 3 protein (CASC3); inositol polyphosphate phosphatase-like 1 (SHIP2); protein diaphanous homolog 1 (DIAP1); ceruloplasmin (CERU); haptoglobin (HPT); complement C3 (CO3); pro-epidermal growth factor (EGF); protein disulfide-isomerase (PDIA1); kappa-casein (CASK); thrombospondin-1 (TSP1); heat shock protein HSP 90-beta (HS90B); complement C4-A (CO4A); receptor-type tyrosine-protein phosphatase alpha (PTPRA); bile salt-activated lipase (CEL); lactoperoxidase (PERL); macrophage mannose receptor 1 (MRC1); tenascin (TENA); xanthine dehydrogenase/oxidase (XDH); paxillin (PAXI); fatty acid synthase (FAS); centromere protein F (CENPF); afadin (AFAD); heterogeneous nuclear ribonucleoprotein K (HNRPK); disks large homolog 4 (DLG4); arginase-2, mitochondrial (ARGI2); tyrosine-protein phosphatase non-receptor type 13 (PTN13); E3 ubiquitin-protein ligase CBL-B (CBLB); protein scribble homolog (SCRIB); dedicator of cytokinesis protein 1 (DOCK1); telomeric repeat-binding factor 2 (TERF2); inverted formin-2 (INF2); programmed cell death protein 4 (PDCD4); E3 ubiquitin-protein ligase UBR4 (UBR4); NMDA receptor-regulated protein 2 (NARG2); 1a-related protein 1 (LARP1); prostate androgen-regulated mucin-like protein 1 (PARM1); ubiquitin carboxyl-terminal hydrolase 51 (UBP51); chromatin complexes subunit BAP18 (BAP18); Armadillo repeat-containing protein 10 (ARM10); misshapen-like kinase 1 (MINK1); protein enabled homolog (ENAH); biorientation of chromosomes in cell division protein 1-like 1 (BD1L1); short transient receptor potential channel 4-associated protein (TP4AP); ankyrin repeat and SAM domain-containing protein 1A (ANS1A); mitogen-activated protein kinase kinase kinase kinase 1 (M4K1); GDP-fucose transporter 1 (FUCT1); E3 ubiquitin-protein ligase UHRF1 (UHRF1); mucin-4 (MUC-4); matrix metalloproteinase-19 (MMP19); serine/threonine-protein kinase 33 (STK33); TRIO and F-actin-binding protein (TARA); apoptotic chromatin condensation inducer in the nucleus (ACINU); UPF0760 protein C2orf29 (CB029); zinc finger protein PLAGL1 (PLAL1); cofilin-2 (COF2); sialic acid-binding Ig-like lectin 9 (SIGL9); protein VPRBP (VPRBP); myosin-4 (MYH4); endoplasmic reticulum mannosyl-oligosaccharide 1,2-alpha-mannosidase (MAN1B1); and cDNA F1157167, highly similar to Etoposide-induced protein 2.4; and the peptide reduces, inhibits or prevents the growth or proliferation of a bacterial organism. In varying embodiments, the peptides are formed by in vivo cleavage by a protease endogenous to mammalian milk, e.g., endogenous to milk from a mammalian species from which the peptides were isolated and/or purified.

In some embodiments, the peptide comprises or consists essentially of a peptide sequence from those listed in Table 1 (e.g., SEQ ID NOs: 1-535) or Table 3. In some embodiments, the peptide comprises and is no longer than a peptide sequence from those listed in Table 1 (e.g., SEQ ID NOs: 1-535) or Table 3.

In some embodiments, the peptide comprises or consists essentially of a subsequence of polymeric immunoglobulin receptor (PIGR) within amino acid positions 550 to 650. In some embodiments, the peptide comprises or consists essentially of a subsequence of polymeric immunoglobulin receptor (PIGR) within amino acid positions selected from 552-571, 577-597 and 598-648. In some embodiments, the PIGR subsequence or peptide comprises from about 9 to about 40 amino acid residues, e.g., from about 9 to about 35 amino acid residues, e.g., from about 9 to about 30 amino acid residues, e.g., about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acid residues. In some embodiments, the PIGR subsequence or peptide comprises or consists essentially of an amino acid sequence selected from the group consisting of AVADTRDQAD; VADTRDQADGSRAS; and DSGSSEEQG. In some embodiments, the PIGR subsequence or peptide comprises or consists essentially of a peptide selected from the group consisting of

YGETAAVYVAVEERKAAGSR; KADAAPDEKVLDSGFREIENK;
AAPDEKVLDSGFREIENK; ADAAPDEKVLDSGFREIENK;
DAAPDEKVLDSGFREIENK; AIQDPRLFAEEKAVADTR;
AIQDPRLFAEEKAVADTRDQADGS; DPRLFAEEKAVADTR;
LFAEEKAVADTRDQADGSR;
LFAEEKAVADTRDQADGSRASVDSGSSEEQGGSSRA; FAEEKAVADTRDQADGSR;
FAEEKAVADTRDQADGSRASVDSGSSEEQGGSSRA; AEEKAVADTRDQADGSR;
EEKAVADTRDQADG; EEKAVADTRDQADGSR; EKAVADTRDQADG;
AVADTRDQAD; AVADTRDQADG; AVADTRDQADGS; AVADTRDQADGSRAS;
AVADTRDQADGSRASVD; AVADTRDQADGSRASVDSG;
AVADTRDQADGSRASVDSGSSEEQG; AVADTRDQADGSRASVDSGSSEEQGG;
AVADTRDQADGSRASVDSGSSEEQGGSS;
AVADTRDQADGSRASVDSGSSEEQGGSSRA;
AVADTRDQADGSRASVDSGSSEEQGGSSRAL;
AVADTRDQADGSRASVDSGSSEEQGGSSRALVST;
AVADTRDQADGSRASVDSGSSEEQGGSSRALVSTLVP;
AVADTRDQADGSRASVDSGSSEEQGGSSRALVSTLVPLG; VADTRDQADGSRAS;
VADTRDQADGSRASVDSGSSEEQGGSS;
VADTRDQADGSRASVDSGSSEEQGGSSRA;
ADTRDQADGSRASVDSGSSEEQGGSSRA; TRDQADGSRASVDSGSSEEQGGSSRA;
DQADGSRASVDSGSSEEQGGSS; DQADGSRASVDSGSSEEQGGSSR;
DQADGSRASVDSGSSEEQGGSSRA; DQADGSRASVDSGSSEEQGGSSRAL;
DQADGSRASVDSGSSEEQGGSSRALVS; DQADGSRASVDSGSSEEQGGSSRALVST;
DQADGSRASVDSGSSEEQGGSSRALVSTLVP;
DQADGSRASVDSGSSEEQGGSSRALVSTLVPL;
DQADGSRASVDSGSSEEQGGSSRALVSTLVPLG;
QADGSRASVDSGSSEEQGGSSRA; DGSRASVDSGSSEEQGGSSR;
DGSRASVDSGSSEEQGGSSRA; ASVDSGSSEEQGGSSRALVSTLVP;
ASVDSGSSEEQGGSSRALVSTLVPLG; SVDSGSSEEQGGSSRA;
SVDSGSSEEQGGSSRALVST; SVDSGSSEEQGGSSRALVSTLVP;
SVDSGSSEEQGGSSRALVSTLVPL; SVDSGSSEEQGGSSRALVSTLVPLG;
VDSGSSEEQGGSSRA; VDSGSSEEQGGSSRALVSTLVP;
VDSGSSEEQGGSSRALVSTLVPLG; DSGSSEEQGGSSRAL; DSGSSEEQGGSSRALV;
DSGSSEEQGGSSRALVST; DSGSSEEQGGSSRALVSTLVP;
DSGSSEEQGGSSRALVSTLVPL; DSGSSEEQGGSSRALVSTLVPLG; AND
GSSEEQGGSSRALV.

In some embodiments, the peptide comprises or consists essentially of a subsequence of beta-casein (CASB) within amino acid positions selected from 16-58, 70-79 and 80-161, and 170-226. In some embodiments, CASB subsequence or peptide comprises from about 6 to about 40 amino acid residues, e.g., from about 6 to about 35 amino acid residues, from about 6 to about 30 amino acid residues, e.g., about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acid residues. In some embodiments, the CASB subsequence or peptide comprises or consists essentially of an amino acid sequence selected from the group consisting of RETIESL; SEESITE; DEHQDKI; PVPQPEI; FDPQIPK; TDLENL; VPQPIP; VLPIPQ; NQELLLNPT; PTHQIYP; QPLAPVH; and HNPISV. In some embodiments, the CASB peptide is selected from the group consisting of RETIESL; RETIESLSS; RETIESLSSSEE; RETIESLSSSEESI; RETIESLSSSEESITE; RETIESLSSSEESITEY; RETIESLSSSEESITEYK; RETIESLSSSEESITEYKQ; RETIESLSSSEESITEYKQK; RETIESLSSSEESITEYKQKVE; RETIESLSSSEESITEYKQKVEK; RETIESLSSSEESITEYKQKVEKV; RETIESLSSSEESITEYKQKVEKVK; RETIESLSSSEESITEYKQKVEKVKHE; RETIESLSSSEESITEYKQKVEKVKHEDQQQG; ETIESLSSSEE; ETIESLSSSEESITE; ETIESLSSSEESITEY; ETIESLSSSEESITEYK; ETIESLSSSEESITEYKQ; ETIESLSSSEESITEYKQK; ETIESLSSSEESITEYKQKVEK; TIESLSSSEESITE; TIESLSSSEESITEY; TIESLSSSEESITEYK; TIESLSSSEESITEYKQKVEK; IESLSSSEESITEYK; ESLSSSEESITE; ESLSSSEESITEYK; SLSSSEESITE; SLSSSEESITEYK; SLSSSEESITEYKQKVEK; LSSSEESITEYK; LSSSEESITEYKQKVEK; SSEESITE; SSEESITEY; SSEESITEYK; SSSEESITE; SSSEESITEYK; SSSEESITEYKQKVE; SSSEESITEYKQKVEK; SEESITE; SEESITEYK; SEESITEYKQKVE; EESITEYKQKV; EESITEYK; ESITEYK; TEYKQKVE; TEYKQKVEKVKHED; QKVEKVK; QKVEKVKHED; QKVEKVKHEDQQQGEDEHQD; QKVEKVKHEDQQQGEDEHQDK; KVEKVKHEDQQQG; KVEKVKHEDQQQGEDEHQDK; VEKVKHEDQQQGEDEHQDK; VEKVKHEDQQQGEDEHQDKIYPS; VKHEDQQQGEDEHQ; VKHEDQQQGEDEHQD; VKHEDQQQGEDEHQDK; VKHEDQQQGEDEHQDKIYP; VKHEDQQQGEDEHQDKIYPS; KHEDQQQGEDEHQD; HEDQQQGEDEHQDK; HEDQQQGEDEHQDKIYP; HEDQQQGEDEHQDKIYPS; DQQQGEDEHQDKIYP; EKVKHEDQQQGEDEHQDK; GEDEHQDK; GEDEHQDKIYPS; DEHQDKI; DEHQDKIYP; VEPIPYGFLPQ; NILPLAQPAVVLPVPQPEIMEVPK; PLAQPAVVLPVPQPEI; AQPAVVLPVPQPEIMEVPK; AQPAVVLPVPQPEIMEVPKAK; AQPAVVLPVPQPEIMEVPKAKDTVYT; AQPAVVLPVPQPEIMEVPKAKDTVYTK; AQPAVVLPVPQPEIMEVPKAKDTVYTKG; QPAVVLPVPQPEI; QPAVVLPVPQPEIM; QPAVVLPVPQPEIMEVPK; QPAVVLPVPQPEIMEVPKA; QPAVVLPVPQPEIMEVPKAK; QPAVVLPVPQPEIMEVPKAKDTVYT; QPAVVLPVPQPEIMEVPKAKDTVYTK; PAVVLPVPQPEI; PAVVLPVPQPEIME; PAVVLPVPQPEIMEVPKAK; PAVVLPVPQPEIMEVPKAKDTVYTKGR; VVLPVPQPEIME; VVLPVPQPEIMEVPK; VVLPVPQPEIMEVPKA; VVLPVPQPEIMEVPKAK; VVLPVPQPEIMEVPKAKDT; VVLPVPQPEIMEVPKAKDTVYT; VVLPVPQPEIMEVPKAKDTVYTK; VVLPVPQPEIMEVPKAKDTVYTKG; VVLPVPQPEIMEVPKAKDTVYTKGR; VLPVPQPEI; VLPVPQPEIM; VLPVPQPEIME; VLPVPQPEIMEVPK; LPVPQPEI; LPVPQPEIM; LPVPQPEIME; LPVPQPEIMEVPK; LPVPQPEIMEVPKA; PVPQPEI; EIMEVPK; EIMEVPKAKDTVYT; MEVPKAKDTVYTKGR; EVPKAKDT; EVPKAKDTVYT; EVPKAKDTVYTK; EVPKAKDTVYTKG; VPKAKDTVYT; VPKAKDTVYTKG; AKDTVYTKGRVMPVLK; KDTVYTKGRVMPVL; KDTVYTKGRVMPVLK; DTVYTKGR; DTVYTKGRV; DTVYTKGRVMPVL; DTVYTKGRVMPVLKGRVMPVLK; GRVMPVLKSPT; GRVMPVLKSPTIP; GRVMPVLKSPTIPFFDPQIPK; GRVMPVLKSPTIPFFDPQIPKLTD; VMPVLKSPTIP; SPTIPFF; SPTIPFFD; SPTIPFFDPQIPK; SPTIPFFDPQIPKL; SPTIPFFDPQIPKLTD; PTIPFFDPQIPKLTD; FFDPQIPK; FDPQIPK; FDPQIPKL; FDPQIPKLT; FDPQIPKLTD; DPQIPKL; DPQIPKLTDLE; DPQIPKLTDLENLHLPLP; PQIPKLTD; PQIPKLTDLENL; TDLENLH; TDLENLHLP; TDLENLHLPLP; DLENLHLP; DLENLHLPLP; LENLHLPLP; LENLHLPLPLLQ; ENLHLPLPLL; ENLHLPLPLLQ; NLHLPLP; HLPLPLL; LLQPLMQQVPQPIPQT; LLQPLMQQVPQPIPQTL; PLMQQVPQPIPQTL; LMQQVPQPIPQT; QQVPQPIP; QVPQPIPQ; QVPQPIPQTL; VPQPIP; VPQPIPQ; SVPQPKVLPIPQQVVPYPQR; SVPQPKVLPIPQQVVPYPQRAVPVQ; SVPQPKVLPIPQQVVPYPQRAVPVQA; VPQPKVLPIPQQV; VLPIPQ; VLPIPQQV; VLPIPQQVVP; VLPIPQQVVPYP; VLPIPQQVVPYPQ; VLPIPQQVVPYPQR; VLPIPQQVVPYPQRA; VLPIPQQVVPYPQRAVPVQ; VLPIPQQVVPYPQRAVPVQA; VLPIPQQVVPYPQRAVPVQAL; LPIPQQVVPYP; LPIPQQVVPYPQRAVP; LPIPQQVVPYPQRAVPVQ; LPIPQQVVPYPQRAVPVQA; PIPQQVVPYPQRAV; PIPQQVVPYPQRAVPVQ; IPQQVVPYPQRAVPVQA; VVPYPQRAVPVQ; VVPYPQRAVPVQA; VPYPQRAVPVQA; AVPVQALLLNQELLLNPTHQIYPVTQPLAPVHNPISV; ALLLNQELLLNPTHQIYPVT; ALLLNQELLLNPTHQIYPVTQPLAPVHNPISV; LLLNQELLLNPTHQIYPVTQ; LLLNQELLLNPTHQIYPVTQPLAPVHNPISV; LLNQELLLNPTHQ; LLNQELLLNPTHQIYPVT; LLNQELLLNPTHQIYPVTQ; LLNQELLLNPTHQIYPVTQPLAPVHNPISV; LNQELLLNPT; LNQELLLNPTHQ; LNQELLLNPTHQIYPVT; LNQELLLNPTHQIYPVTQPLAPVHNPISV; NQELLLNPT; NQELLLNPTHQIYP; NQELLLNPTHQIYPVT; NQELLLNPTHQIYPVTQ; NQELLLNPTHQIYPVTQPLAPVH; NQELLLNPTHQIYPVTQPLAPVHNPISV; QELLLNPTHQIYP; QELLLNPTHQIYPVT; QELLLNPTHQIYPVTQPLAPVHNPISV; ELLLNPTHQIYP; ELLLNPTHQIYPVT; ELLLNPTHQIYPVT; ELLLNPTHQIYPVTQPLAPVHNPISV; LLLNPTHQIYP; LLLNPTHQIYPVT; LLLNPTHQIYPVTQ; LLLNPTHQIYPVTQPLAP; LLLNPTHQIYPVTQPLAPVH; LLLNPTHQIYPVTQPLAPVHNPISV; LLNPTHQIYP; LLNPTHQIYPVTQPLAPVH; LLNPTHQIYPVTQPLAPVHNPIS; LLNPTHQIYPVTQPLAPVHNPISV; LNPTHQIYPVTQ; LNPTHQIYPVTQPLAPVHNPISV; NPTHQIYPVTQ; NPTHQIYPVTQPLAPVHNPISV; PTHQIYPVTQ; PTHQIYPVTQPLAPVHNPISV; THQIYPVTQPLAPVHNPISV; HQIYPVTQPLAPVHNPISV; QIYPVTQPLAPVHNPISV; IYPVTQPLAPVHNPISV; YPVTQPLAPVH; YPVTQPLAPVHNPISV; PVTQPLAPVHNPISV; VTQPLAPVHNPISV; TQPLAPVH; TQPLAPVHNPISV; QPLAPVH; QPLAPVHNPISV; PLAPVHNPISV; APVHNPISV; PVHNPISV; and HNPISV. In varying embodiments, the CASB subsequence or peptide subsequence or peptide comprises and is no longer than an amino acid sequence selected from the group consisting of GRVMPVLKSPTIPFFDPQIPK; PTIPFFDPQIPKLTD; SPTIPFFDPQIPK; SPTIPFFDPQIPKL; SPTIPFFDPQIPKLTD; FDPQIPK; GRVMPVLKSPTIPFFDPQIPKLTD; AVPVQALLLNQELLLNPTHQIYPVTQPLAPVHNPISV; ALLLNQELLLNPTHQIYPVTQPLAPVHNPISV; ELLLNPTHQIYPVTQPLAPVHNPISV; ELLLNPTHQIYPVT; ELLLNPTHQIYPVTQ; HQIYPVTQPLAPVHNPISV; LLLNPTHQIYPVT; LLLNPTHQIYPVTQ; LLLNPTHQIYPVTQPLAP; LLLNPTHQIYPVTQPLAPVH; LLLNPTHQIYPVTQPLAPVHNPISV; LLLNQELLLNPTHQIYPVTQPLAPVHNPISV; LLNPTHQIYPVTQPLAPVH; LLNPTHQIYPVTQPLAPVHNPIS; LLNPTHQIYPVTQPLAPVHNPISV; LLNQELLLNPTHQIYPVT; LLNQELLLNPTHQIYPVTQ; LLNQELLLNPTHQIYPVTQPLAPVHNPISV; LLNQELLLNPTHQ; LNPTHQIYPVTQ; LNPTHQIYPVTQPLAPVHNPISV; LNQELLLNPT; LNQELLLNPTHQ; LNQELLLNPTHQIYPVTQPLAPVHNPISV; NQELLLNPT; NQELLLNPTHQIYP; NQELLLNPTHQIYPVT; NQELLLNPTHQIYPVTQ; NQELLLNPTHQIYPVTQPLAPVH; NQELLLNPTHQIYPVTQPLAPVHNPISV; QELLLNPTHQIYP; QELLLNPTHQIYPVT; QELLLNPTHQIYPVTQPLAPVHNPISV; YPVTQPLAPVH; YPVTQPLAPVHNPISV; NPTHQIYPVTQ; NPTHQIYPVTQPLAPVHNPISV; PLAPVHNPISV; PTHQIYPVTQPLAPVHNPISV; PVHNPISV; PVTQPLAPVHNPISV; QPLAPVHNPISV; THQIYPVTQPLAPVHNPISV; TQPLAPVHNPISV; VTQPLAPVHNPISV; APVHNPISV; QIYPVTQPLAPVHNPISV; IYPVTQPLAPVHNPISV; LNQELLLNPTHQIYPVT; QPLAPVH; LLLNPTHQIYPVT; LLLNPTHQIYPVTQPLAP; LLLNPTHQIYP; ELLLNPTHQIYPVT; and LLNQELLLNPTHQIYPVTQ. In varying embodiments, the CASB subsequence or peptide does not comprise a sequence selected from the group consisting of QPTIPFFDPQIPK (SEQ ID NO:505) and QELLLNPTHQYPVTQPLAPVHNPISV (SEQ ID NO:506).

In some embodiments, the peptide comprises or consists essentially of a subsequence of butyrophilin subfamily 1 member A1 (BT1A1) within amino acid positions selected from 27-40, 79-108, 415-418 and 477-526. In some embodiments, the BT1A1 subsequence or peptide comprises from 6 to 35 amino acid residues, e.g., from about 6 to about 30 amino acid residues, from about 6 to about 25 amino acid residues, e.g., about 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acid residues. In some embodiments, the BT1A1 subsequence or peptide comprises or consists essentially of an amino acid sequence selected from the group consisting of DVIGPP; GREQEAEQMPEYR; TLVQDGIAK; KEIPLSPMGED; IPLSPMGEDS; and SKLIPTQPSQG. In some embodiments, the BT1A1 peptide is selected from the group consisting of APFDVIGPPEPILA; DVIGPP; DGREQEAEQMPEY; DGREQEAEQMPEYR; DGREQEAEQMPEYRG; DGREQEAEQMPEYRGR; GREQEAEQMPEYR; GREQEAEQMPEYRGR; GRATLVQDGIAK; GRATLVQDGIAKGRVA; TLVQDGIAK; TLVQDGIAKGRVA; LPLAGP; DGPERVTVIANAQDLS; QDLSKEIPLSPMGEDSAPRDADTLH; KEIPLSPMGED; KEIPLSPMGEDSAPR; KEIPLSPMGEDSAPRDADT; KEIPLSPMGEDSAPRDADTLH; KEIPLSPMGEDSAPRDADTLHS; KEIPLSPMGEDSAPRDADTLHSK; KEIPLSPMGEDSAPRDADTLHSKLIPTQPSQ; KEIPLSPMGEDSAPRDADTLHSKLIPTQPSQGAP; EIPLSPMGEDSAPR; EIPLSPMGEDSAPRDADTLH; IPLSPMGEDS; IPLSPMGEDSAPR; IPLSPMGEDSAPRDADTLH; SPMGEDSAPRDADTLH; EDSAPRDADTLH; APRDADTLHSKLIPTQPSQGAP; ADTLHSKLIPTQPSQGAP; SKLIPTQPSQG; and SKLIPTQPSQGAP.

In some embodiments, the peptide comprises or consists essentially of a subsequence of alpha-S1-casein (CASA1) within amino acid positions selected from 16-68, 70-79 and 175-183. In some embodiments, the CASA1 subsequence or peptide comprises from 7 to 35 amino acid residues, e.g., from about 7 to about 30 amino acid residues, from about 7 to about 25 amino acid residues, e.g., about 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acid residues. In some embodiments, the CASA1 subsequence or peptide comprises or consists essentially of an amino acid sequence selected from the group consisting of RPKLPLR; RLQNPSE; NPSESSEPIP and NILREKQTDE. In some embodiments, the CASA1 peptide is selected from the group consisting of RPKLPLR; RPKLPLRYPE; RPKLPLRYPERLQ; RPKLPLRYPERLQNPSESSEPIPLESREEYMNGMN; RLQNPSE; RLQNPSESSEPIP; RLQNPSESSEPIPLE; RLQNPSESSEPIPLESR; RLQNPSESSEPIPLESREEYMNGM; RLQNPSESSEPIPLESREEYMNGMN; RLQNPSESSEPIPLESREEYMNGMNR; LQNPSESSEPIPLE; LQNPSESSEPIPLESR; LQNPSESSEPIPLESREEYMNGMN; NPSESSEPIP; NPSESSEPIPLES; NPSESSEPIPLESREEYMNGMN; MNRQRNILR; QRNILREKQTDEIKDTR; NILREKQTDE; NILREKQTDEIKDTR; EKQTDEIKDTR; NYEKNNVML; and YEKNNVML.

In some embodiments, the peptide comprises or consists essentially of a subsequence of osteopontin (OSTP) within amino acid positions selected from 17-25, 34-42, 155-216, 155-168, 169-203, 206-216, 232-246, and 303-314. In some embodiments, the OSTP subsequence or peptide comprises from 6 to 35 amino acid residues, e.g., from about 6 to about 30 amino acid residues, from about 6 to about 25 amino acid residues, e.g., about 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acid residues. In some embodiments, the OSTP subsequence or peptide comprises or consists essentially of an amino acid sequence selected from the group consisting of IPVKQADS; GDSVVYGLR; EDITSH; and IPVAQD. In some embodiments, the OSTP peptide is selected from the group consisting of IPVKQADS; IPVKQADSG; NKYPDAVAT; TYDGRGDSVVYGLR; GDSVVYGLR; SKSKKFRRPDIQYPDATD; SKSKKFRRPDIQYPDATDEDITSH; SKSKKFRRPDIQYPDATDEDITSHMESEELNGAYK; RPDIQYPDATD; RPDIQYPDATDEDIT; RPDIQYPDATDEDITSH; RPDIQYPDATDEDITSHMESEELNGAYK; RRPDIQYPDATDEDIT; RRPDIQYPDATDEDITSH; RRPDIQYPDATDEDITSHMESEELNGAYK; DIQYPDATDEDITSH; DIQYPDATDEDITSHMESEELNGAYK; YPDATDEDITSH; ATDEDITSH; ATDEDITSHMESEELNGAYK; EDITSHM; EDITSHME; EDITSHMESEELNGAYK; ESEELNGAYK; SEELNGAYK; AIPVAQDLNAPS; AIPVAQDLNAPSD; IPVAQD; IPVAQDLNAPS; DDQSAETHSHKQSRLY; DQSAETHSHKQSRLY; RISHELDSASSEVN; ISHELDSASSEVN; SHELDSASSEVN; and HELDSASSEVN.

In some embodiments, the peptide comprises or consists essentially of a subsequence of perilipin-2 (PLIN2) within amino acid positions selected from 66-77, 137-145, 171-181, and 417-437. In some embodiments, the PLIN2 subsequence or peptide comprises from 6 to 25 amino acid residues, e.g., from about 6 to about 20 amino acid residues, from about 6 to about 15 amino acid residues, e.g., about 6, 7, 8, 9, 10, 11, 12, 13, 14, 15, 16, 17, 18, 19, 20, 21, 22, 23, 24 or 25 amino acid residues. In some embodiments, the PLIN2 subsequence or peptide comprises or consists essentially of an amino acid sequence selected from the group consisting of LPIIQKLEPQ and EMDKSSQETQRSEHKTH. In some embodiments, the PLIN2 peptide is selected from the group consisting of LPIIQKLEPQ; LPIIQKLEPQIA; VMDKTKGAV; LVSSGVENALT; DQGAEMDKSSQETQRSEHKTH; AEMDKSSQETQRSEHKTH; and EMDKSSQETQRSEHKTH.

In embodiments, the peptide comprises or consists essentially of a subsequence of mucin-1 (MUC1) within amino acid positions selected from 1223-1255. In some embodiments, the MUC1 subsequence or peptide comprises from 10 to 35 amino acid residues, e.g., from about 10 to about 30 amino acid residues, from about 10 to about 25 amino acid residues, e.g., about 10, 11, 12, 13, 14, 15, 16, 17, 18, 19, 20, 21, 22, 23, 24 or 25 amino acid residues. In some embodiments, the MUC 1 subsequence or peptide comprises or consists essentially of an amino acid sequence selected from the group consisting of SPYEKVSAGNGGSS and TNPAVAATSANL. In some embodiments, the MUC1 peptide is selected from the group consisting of STDRSPYEKVSAGNGGSSLSY; TDRSPYEKVSAGNGGSSLS; TDRSPYEKVSAGNGGSSLSY; TDRSPYEKVSAGNGGSSLSYTNPAVAATSANL; DRSPYEKVSAGNGGSSLS; SPYEKVSAGNGGSS; SPYEKVSAGNGGSSL; SPYEKVSAGNGGSSLS; and TNPAVAATSANL.

In some embodiments, the peptide comprises or consists essentially of a subsequence of kappa-casein (CASK) within amino acid positions selected from 79-109 and 172-180. In some embodiments, the CASK subsequence or peptide comprises from 7 to 20 amino acid residues, e.g., from about from about 7 to about 15 amino acid residues, from about 7 to about 15 amino acid residues, e.g., about 6, 7, 8, 9, 10, 11, 12, 13, 14, 15, 16, 17, 18, 19 or 20 amino acid residues. In some embodiments, the CASK peptide is selected from the group consisting of TYYANPAVVRPHA, TYYANPAVVRPHAQIP, TYYANPAVVRPHAQIPQR, TYYANPAVVRPHAQIPQRQY, YANPAVVRPHAQIPQR, ANPAVVRPHAQIPQRQY, LPNSHPPT, LPNSHPPTV, LPNSHPPTVVR, HPPTVVR and TTTVAVTPP. In varying embodiments, the CASK subsequence or peptide comprises and is no longer than an amino acid sequence selected from the group consisting of LPNSHPPTVVR; TYYANPAVVRPHA; TYYANPAVVRPHAQIP; ANPAVVRPHAQIPQRQY; LPNSHPPTV; HPPTVVR; LPNSHPPT; TYYANPAVVRPHAQIPQR; TYYANPAVVRPHAQIPQRQY and YANPAVVRPHAQIPQR. In varying embodiments, the CASK subsequence or peptide does not comprise YQRRPAIAINNPYVPRTYYANPAVVRPHAQIPQRQYLPNSHPPTVVRRPNLHPSF (SEQ ID NO:504).

In some embodiments, the peptide comprises one or more modifications selected from the group consisting of:

i) oxidation or dioxidation of one or more methionine (M) residues;

ii) deamination of one or more glutamine (Q) residues; and/or

iii) phosphorylation of one or more serine (S), threonine (T) or tyrosine (Y) residues.

In some embodiments, the peptide comprises one or more modifications selected from the group consisting of:

i) one or more of the amino acid residues are D-amino acids;

ii) the peptide comprises protecting groups at one or both of the N-terminus or the C terminus; iii) the peptide is fully or partially retro-inverso; and/or

iv) the peptide is circularized.

In some embodiments, the peptide further comprises from 1 to 5 flanking amino acid residues at the amino and/or carboxyl termini. In some embodiments, the peptide further comprises a cysteine residue at the amino terminus and a cysteine residue at the carboxyl terminus.

In some embodiments, the peptide reduces, inhibits or prevents the growth or proliferation of a bacterial organism selected from the group consisting of Escherichia coli, Staphylococcus aureus, Streptococcus agalactiae, Streptococcus uberis, Serratia marcescens and Coagulase-negative staphylococcus (CNS).

In a further aspect, polypeptides comprising two or more peptides, e.g., 2, 3, 4, 5, 6, 7, 8, 9, 10, 20, 30, 40, 50, 60, 70, 80, 90, 100, 150, 200, 250, 300, 350, 400, 450, 500, 503 peptides, described above and herein, are provided. In some embodiments, the two or more peptides are conjugated. In some embodiments, the polypeptide is a fusion protein comprising of two or more peptides, as described above and herein.

In a related aspect, the invention provides compositions comprising one or more peptides or one or more polypeptides, e.g., 1, 2, 3, 4, 5, 6, 7, 8, 9, 10, 20, 30, 40, 50, 60, 70, 80, 90, 100, 150, 200, 250, 300, 350, 400, 450, 500, 503 peptides, described herein (e.g., of Table 1; e.g., SEQ ID NOs:1-535 or Table 3), and a pharmaceutically acceptable carrier. Embodiments of the peptides and polypeptides are as described above and herein. In some embodiments, the composition is formulated for topical administration. In some embodiments, the composition is formulated for oral administration. In some embodiments, the composition is formulated for intra-ductal administration or for administration directly into the mammary gland. In some embodiments, the composition is formulated for administration to the site of infection.

In another aspect, the invention provides methods of reducing, inhibiting or preventing the growth or proliferation of a bacterial organism, comprising contacting the bacterial organism with one or more peptides or one or more polypeptide, as described above and herein. In varying embodiments, the bacterial organism selected from the group consisting of Escherichia coli and Staphylococcus aureus. The methods can be performed in vivo or in vitro. In some embodiments, the bacterial organism is in a host subject. In some embodiments, the host subject is a human. In some embodiments, the host subject has a bacterial infection treatable by topical administration of the peptide(s) or polypeptide(s). In some embodiments, the host subject has a bacterial infection of the oral cavity. In some embodiments, the host subject has a bacterial infection of the mammary gland and/or the mammary duct. In some embodiments, the host subject has a bacterial infection of the skin.

In a further aspect, the invention provides methods for reducing, preventing, inhibiting and/or mitigating a bacterial infection of the mammary gland in a lactating mammal, comprising administering to a mammary gland of the lactating mammal a therapeutically effective amount of at least one peptide or a mixture of peptides, as described herein, or a polypeptide comprising one or more antibacterial peptides, described herein, or a composition comprising one or more antibacterial peptides, described herein. In varying embodiments, the lactating mammal is a human. In some embodiments, the peptide, polypeptide or composition is administered orally, topically or into the mammary duct.

In another aspect, the invention provides methods for reducing, preventing, inhibiting and/or mitigating a bacterial infection in the oral cavity of a nursing mammal, comprising administering to the oral cavity of the nursing mammal a therapeutically effective amount of at least one peptide or a mixture of peptides, as described herein, or a polypeptide comprising one or more antibacterial peptides, described herein, or a composition comprising one or more antibacterial peptides, described herein. In varying embodiments, the nursing mammal is a human. In some embodiments, the peptide, polypeptide or composition is administered orally or topically.

### DEFINITIONS

The term “contacting” includes reference to placement in direct physical association.

As used herein, “polypeptide”, “peptide” and “protein” are used interchangeably and include reference to a polymer of amino acid residues. As used herein, the term “peptide” is used in its broadest sense to refer to conventional peptides (i.e. short polypeptides containing L or D-amino acids), as well as peptide equivalents, peptide analogs and peptidomimetics that retain the desired functional activity. Peptide equivalents can differ from conventional peptides by the replacement of one or more amino acids with related organic acids (such as PABA), amino acids or the like, or the substitution or modification of side chains or functional groups. The terms apply to amino acid polymers in which one or more amino acid residue is an artificial chemical analogue of a corresponding naturally occurring amino acid, as well as to naturally occurring amino acid polymers. The terms also apply to polymers containing conservative amino acid substitutions such that the protein remains functional.

The terms “peptide equivalents”, “peptide analogs”, “peptide mimetics”, and “peptidomimetics” are used interchangeably unless specified otherwise. Peptide analogs are commonly used in the pharmaceutical industry as non-peptide drugs with properties analogous to those of the template peptides. (Fauchere, J. (1986) Adv. Drug Res. 15: 29; Veber and Freidinger (1985) TINS p. 392; and Evans et al. (1987) J. Med. Chem 30: 1229). Peptide analogs are usually developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biological or pharmacological activity), such as naturally-occurring receptor-binding polypeptide, but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends Pharm Sci (1980) pp. 463-468 (general review); Hudson, D. et al., Int J Pept Prot Res (1979) 14:177-185 (—CH₂NH—, CH₂CH₂—); Spatola, A. F. et al., Life Sci (1986) 38:1243-1249 (—CH₂S); Hann, M. M., J Chem Soc Perkin Trans I (1982) 307-314 (—CH—CH—, cis and trans); Almquist, R. G. et al., J Med Chem (1980) 23:1392-1398 (—COCH₂—); Jennings-White, C. et al., Tetrahedron Lett (1982) 23:2533 (—COCH₂—); Szelke, M. et al., European Appln. EP 45665 (1982) CA: 97:39405 (1982) (—CH(OH)CH₂—); Holladay, M. W. et al., Tetrahedron Lett (1983) 24:4401-4404 (—C(OH)CH₂—); and Hruby, V. J., Life Sci (1982) 31:189-199 (—CH₂S—). Portions or all of the peptide backbone can also be replaced by conformationally constrained cyclic alkyl or aryl substituents to restrict mobility of the functional amino acid sidechains specified herein as described in the following references: 1. Bondinell et al. Design of a potent and orally active nonpeptide platelet fibrinogen receptor (GPIIb/IIIa) antagonist. Bioorg Med Chem 2:897 (1994). 2. Keenan et al. Discovery of potent nonpeptide vitronectin receptor (alpha v beta 3) antagonists. J Med Chem 40:2289 (1997). 3. Samanen et al. Potent, selective, orally active 3-oxo-1,4-benzodiazepine GPIIb/IIIa integrin antagonists. J Med Chem 39:4867 (1996).

The peptides of this invention may be produced by recognized methods, such as recombinant and synthetic methods that are well known in the art. Recombinant techniques are generally described in Sambrook, et al., Molecular Cloning: A Laboratory Manual, (3rd ed.) Vols. 1-3, Cold Spring Harbor Laboratory, (2001). Techniques for the synthesis of peptides are well known and include those described in Merrifield, J. Amer. Chem. Soc. 85:2149-2456 (1963), Atherton, et al., Solid Phase Peptide Synthesis: A Practical Approach, IRL Press (1989), and Merrifield, Science 232:341-347 (1986).

The term “residue” or “amino acid residue” or “amino acid” includes reference to an amino acid that is incorporated into a protein, polypeptide, or peptide (collectively “peptide”). The amino acid can be a naturally occurring amino acid and, unless otherwise limited, can encompass known analogs of natural amino acids that can function in a similar manner as naturally occurring amino acids.

The amino acids and analogs referred to herein are described by shorthand designations as follows in Table A:

TABLE A
Amino Acid Nomenclature

| Name | 3-letter | 1-letter |
|---|---|---|
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Homoserine | Hse | — |
| Isoleucine | Ile | I |
| Leucine | Leu | L |
| Lysine | Lys | K |
| Methionine | Met | M |
| Methionine sulfoxide | Met (O) | — |
| Methionine | Met (S—Me) | — |
| methylsulfonium | | |
| Norleucine | Nle | — |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution”, when describing a protein refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well known in the art. The following six groups in Table B each contain amino acids that are conservative substitutions for one another:

TABLE B

1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).
See also, Creighton, Proteins: Structures and Molecular Properties, W. H. Freeman and Company, New York (2nd Ed., 1992).

The terms “identical” or percent “identity,” and variants thereof in the context of two or more polypeptide sequences, refer to two or more sequences or subsequences that are the same. Sequences are “substantially identical” if they have a specified percentage of amino acid residues or nucleotides that are the same (i.e., at least 60% identity, optionally at least 65%, 70%, 75%, 80%, 85%, 90%, 95%, 96%, 97%, 98%, or 99% identity to a reference sequence (e.g., the peptides of Table 1; SEQ ID NOs: 1-535; or Table 3) over a specified region (or the whole reference sequence when not specified)), when compared and aligned for maximum correspondence over a comparison window, or designated region as measured using one of the following sequence comparison algorithms or by manual alignment and visual inspection. The present invention provides polypeptides substantially identical to the polypeptides listed in Table 1 (e.g., SEQ ID NOs: 1-535) or Table 3. Optionally, the identity exists over a region that is at least about 5, 10, 15, 20, 25, 30, 35, 40, 45, 50, 55, 60 amino acids in length, or over the full-length of the sequence.

The terms “similarity,” or “percent similarity,” and variants thereof in the context of two or more polypeptide sequences, refer to two or more sequences or subsequences that have a specified percentage of amino acid residues that are either the same or similar to a reference sequence (e.g., SEQ ID NOs: 1-535) as defined in the conservative amino acid substitutions defined above (i.e., 60%, optionally 65%, 70%, 75%, 80%, 85%, 90%, 95%, 96%, 97%, 98% or 99% similar over a specified region), when compared and aligned for maximum correspondence over a comparison window, or designated region as measured using one of the following sequence comparison algorithms or by manual alignment and visual inspection. Sequences having less than 100% similarity but that have at least one of the specified percentages are said to be “substantially similar.” Optionally, this identity exists over a region that is at least about 5, 10, 15, 20, 25, 30, 35, 40, 45, 50, 55, 60 amino acids in length, or over the full-length of the sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

The term “comparison window”, and variants thereof, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can also be conducted by the local homology algorithm of Smith and Waterman Add. APL. Math. 2:482 (1981), by the homology alignment algorithm of Needle man and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), Karlin and Altschul Proc. Natl. Acad. Sci. (U.S.A.) 87:2264-2268 (1990), or by manual alignment and visual inspection (see, e.g., Ausubel et al., Current Protocols in Molecular Biology (1995 supplement)).

Examples of an algorithm that is suitable for determining percent sequence identity and sequence similarity include the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Standard BLAST algorithm parameters have an expected threshold of 10 (according to the stochastic model of Karlin and Altschul (PNAS, 87:2264-2268 (1990)); a word size of 28; reward and penalty of 1/−2 (a ratio of 0.5, or 1/−2, is used for sequences that are 95% conserved); and a linear GAP cost.

As used herein, the term “retro-inverso peptide” refers to a peptide that typically comprises the same amino acid sequence as a peptide having L-amino acids, but whose sequence is comprised partially or entirely of D-amino acids, thus having a reversed stereochemistry from a peptide which is synthesized using L-amino acids. By constructing a peptide using the D-amino acids in inverse order (i.e. the sequences are denoted from left to right, from C-terminal to N-terminal amino acid as opposed to from N-terminal to C-terminal as written or denoted in the case of L-amino acids; see infra), one obtains a retro-inverso peptide that restores the same stereochemistry for the side chains as the parent L-amino acid peptide. Use of retro-inverso peptide sequences minimizes enzymatic degradation and, therefore, extends biological half-life of the peptide moiety. Also, these sequences may favorably alter potential immunogenic properties of the analogous conjugates prepared from normal L-amino acid sequences. The retro-inverso sequences (as free peptides or conjugates) are particularly useful in those applications that require or prefer orally active agents (due to resistance to enzymolysis). For the purposes of the present invention, retro-inverso peptides are denoted by “ri”, and are written, from left to right, from the C-terminal to the N-terminal amino acid, e.g. the opposite of typical L-peptide notation. In one embodiment, the retro-inverso peptide of the present invention incorporates all D isomer amino acids. When the retro-inverso peptide incorporate all D isomer amino acids, it is termed a “D-reverse peptide”.

The terms “substantially pure,” or “isolated” when used to describe peptides or a mixture of peptides (e.g., one or more peptides of Table 1 (e.g., SEQ ID NOs:1-535) or Table 3, described herein), refers to a peptide separated from proteins or other contaminants with which they are naturally associated or with which they are associated, e.g., in mammalian milk. In one embodiment, a peptide or mixture of peptides makes up at least 50% of the total polypeptide content of the composition containing the peptide or mixture of peptides, and in one embodiment, at least 60%, in one embodiment, at least 75%, in one embodiment at least 90%, and in one embodiment, at least 95% of the total polypeptide content. The term “purified” denotes that a peptide or mixture of peptides (e.g., one or more peptides of Table 1 (e.g., SEQ ID NOs:1-535) or Table 3, described herein) gives rise to essentially one band in an electrophoretic gel. Particularly, it means that the peptide or mixture of peptides is at least 80%, 85% or 90% pure, more preferably at least 95% pure, and most preferably at least 99% pure.

The term “isolated,” and variants thereof when applied to a peptide or mixture of peptides (e.g., one or more peptides of Table 1 (e.g., SEQ ID NOs:1-535) or Table 3, described herein), denotes that the peptide or mixture of peptides is essentially free of other non-peptide components with which it is associated in the natural state (e.g., in mammalian milk). The peptide or mixture of peptides can be in either a dry or aqueous solution. Purity and homogeneity are typically determined using known techniques, such as polyacrylamide gel electrophoresis or high performance liquid chromatography. A peptide or mixture of peptides that is the predominant species present in a preparation is substantially purified.

The terms “conjugating,” “joining,” “bonding” or “linking” refer to making two polypeptides into one contiguous polypeptide molecule. In the context of the present invention, the terms include reference to joining an antibody moiety to an effector molecule (EM). The linkage can be either by chemical or recombinant means. Chemical means refers to a reaction between the antibody moiety and the effector molecule such that there is a covalent bond formed between the two molecules to form one molecule.

The term “in vivo” includes reference to inside the body of the organism from which the cell was obtained. “Ex vivo” and “in vitro” means outside the body of the organism from which the cell was obtained.

As used herein, “mammalian cells” includes reference to cells derived from mammals including humans, rats, mice, guinea pigs, chimpanzees, or macaques. The cells may be cultured in vivo or in vitro.

The terms “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other healthworker in a hospital, as an outpatient, or other clinical context. In certain embodiments the subject may not be under the care or prescription of a physician or other healthworker.

As used herein, “administering” refers to local and systemic administration, e.g., including enteral, parenteral, pulmonary, and topical/transdermal administration. Routes of administration for compounds (e.g., tropisetron, disulfuram, honokiol and/or nimetazepam) that find use in the methods described herein include, e.g., oral (per os (P.O.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arterial, intradermal, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

As used herein, the term “topical administration” refers to administration onto any accessible body surface of any mammalian species, preferably the human species, for example, the skin, the oral cavity or the outer surface of the eye. Suitable pharmaceutically-acceptable carriers for topical application include those suitable for use in liquids (including solutions and lotions), creams, gels, and the like. The composition can be packaged in a form suitable for metered application, such as in container equipped with a dropper.

The term “co-administer” refers to the simultaneous presence of two active agents in the blood of an individual. Active agents that are co-administered can be concurrently or sequentially delivered.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

The terms “effective amount” or “amount effective to” or “therapeutically effective amount” includes reference to a dosage of a therapeutic agent sufficient to produce a desired result, such as inhibiting, reducing or preventing bladder cancer cell growth or tumor growth; promoting bladder tumor reduction or elimination; or blocking, reducing, inhibiting or preventing bladder cancer growth, migration or metastasis. The term “effective amount” as used in relation to pharmaceutical compositions, typically refers to the amount of the active ingredient, e.g. the peptides of the invention, which are required to achieve the desired goal. For example, in therapeutic applications, an effective amount will be the amount required to be administered to a patient to result in treatment of the particular disorder for which treatment is sought (e.g., bladder cancer). The term “treatment of a disorder” denotes the reduction or elimination of symptoms of a particular disorder. Effective amounts will typically vary depending upon the nature of the disorder, the peptides used, the mode of administration, and the size and health of the patient. In one embodiment, the effective amount of the peptides of the invention ranges from 1 μg to 1 g of peptide for a 70 kg patient, and in one embodiment, from 1 μg to 10 mg. In one embodiment, the concentration of peptide (or peptide analog) administered ranges from 0.1 μM to 10 mM, and in one embodiment, from 5 μM to 1 mM, in one embodiment, from 5 μM to 100 μM, and in one embodiment from 5 μM to 40 μM.

As used herein, the terms “treating” and “treatment” refer to delaying the onset of, retarding or reversing the progress of, or alleviating or preventing either the disease or condition to which the term applies (e.g., bacterial infection), or one or more symptoms of such disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease (e.g., bacterial infection).

The terms “inhibiting,” “reducing,” “decreasing” with respect to bacterial growth or proliferation refers to inhibiting the growth, spread of a bacterial infection in a subject by a measurable amount using any method known in the art. The growth, progression or spread of a bacterial infection is inhibited, reduced or decreased if the bacterial cell burden is at least about 10%, 20%, 30%, 50%, 80%, or 100% reduced in comparison to the bacterial cell burden prior to administration of one or more peptides of Table 1 (e.g., SEQ ID NOs:1-535) or Table 3. In some embodiments, the growth, progression or spread of a bacterial infection is inhibited, reduced or decreased by at least about 1-fold, 2-fold, 3-fold, 4-fold, or more in comparison to the bacterial cell burden prior to administration of the one or more peptides of Table 1 (e.g., SEQ ID NOs:1-535) or Table 3.

As used herein the term “mastitis” refers to an inflammation of a mammary gland or an udder, caused by a physical injury, introduction of chemicals, viruses, fungus, parasites or, most commonly, bacterial invasion and their toxins. “Mastitis” is used to describe all forms of such inflammation, including subclinical and clinical mastitis, clinical mastitis including mild, severe and chronic mastitis.

In subclinical mastitis, no swelling of the breast or udder is detected nor is there observable abnormalities in the milk. Special screening tests, however, such as the California Mastitis Test (CMT), Wisconsin Mastitis Test (WMT) based on an estimation of somatic cell counts and the catalase test will show changes in the milk composition. This type of mastitis is commonly referred to as “hidden.”

Clinical mastitis can be mild or acute, and is characterized by the presence of leukocytes in the milk. Mild clinical mastitis involves changes in the milk appearance including presence of flakes or clots, watery milk or other unusual forms of the milk. Mild clinical mastitis may be accompanied by other symptoms including hot, sensitive or swollen breast or udder.

Severe clinical mastitis involves the symptoms of hot, sensitive, firm breast or udder that is quite painful to the lactating animal. The onset of severe clinical mastitis is sudden and the lactating animal may become ill showing signs of fever, rapid pulse, depression, weakness and loss of appetite. When the whole lactation system of the animal is affected, the condition is referred to as acute systemic mastitis. The severe symptoms may be also accompanied with cessation of milk production.

As used herein, the phrase “consisting essentially of” refers to the genera or species of active pharmaceutical agents recited in a method or composition, and further can include other agents that, on their own do not substantial activity for the recited indication or purpose. In some embodiments, the phrase “consisting essentially of” expressly excludes non-peptide components of mammalian milk. In some embodiments, the phrase “consisting essentially of” expressly excludes peptides or polypeptides containing and longer than the sequence of the recited peptide (e.g., longer peptides and/or the full-length polypeptide).

### BRIEF DESCRIPTION OF THE DRAWINGS

FIGS. 1A-C illustrate assays demonstrating inhibition of S. aureus growth performed in triplicate using different number of bacteria for the inoculation. A. Assay 1, 10⁶ initial bacteria. B. Assay 2, 10⁵ initial bacteria. C. Assay 3, 10⁴ initial bacteria.

FIGS. 2A-C illustrate assays demonstrating inhibition of E. coli growth performed in triplicate using different number of bacteria for the inoculation. A. Assay 1, 10⁶ initial bacteria. B. Assay 2, 10⁵ initial bacteria. C. Assay 3, 10⁴ initial bacteria.

FIG. 3 illustrates 1-dimensional SDS-PAGE on the various peptide extractions. Gel 1 was performed with 50 μg in each lane. Gel 2 was performed with 10 μg per lane.

FIG. 4 illustrates example extracted compound chromatograms from identified peptides. Polyimmunoglobulin receptor, PIGR; β-casein, B-CN; immunoglobulin gamma-1 chain C region, IgG; butyrophilin, BTN.

FIG. 5 illustrates example tandem mass spectrum of peptide RETIESLSSEESITEYK from B-CN identified by both X!Tandem and MS-GFDB.

FIG. 6 illustrates a Venn diagram of the number of unique peptides found in MS-GFDB and X!Tandem.

FIG. 7 illustrates unique peptides identified by protein of origin. PIGR: polymeric immunoglobulin receptor; BSAL: Bile salt-activated lipase; MMR1: Macrophage mannose receptor 1; MLK1: Misshapen-like kinase 1; SBIGL-9: Sialic acid-binding Ig-like lectin 9; PRB1P: Proteins represented by one peptide.

FIG. 8A-B illustrate peptides corresponding to amino acid residues 552-648 (e.g., 552-571, 577-597 and 598-648) of polymeric immunoglobulin receptor (PIGR) (Uniprot code sp|P01833|PIGR_HUMAN).

FIGS. 9A-F illustrate peptides corresponding to amino acid residues 16-58, 70-79, 80-161 and 170-226 of beta-casein (Uniprot code sp|P05814|CASB_HUMAN).

FIG. 10 illustrates peptides corresponding to amino acid residues 27-40, 79-108, 415-418 and 477-526 of butyrophilin subfamily 1 member A1 (sp|Q13410|BT1A1_HUMAN).

FIG. 11 illustrates peptides corresponding to amino acid residues 16-68 and 175-185 of alpha-S1-casein (Uniprot code sp|P47710|CASA1_HUMAN).

FIG. 12 illustrates peptides corresponding to amino acid residues 17-25, 34-42, 155-168, 169-203, 204-216, 232-246 and 303-314 of osteopontin (Uniprot code sp|P10451|OSTP_HUMAN).

FIG. 13 illustrates peptides corresponding to amino acid residues 66-77, 137-145, 171-181 and 417-437 of perilipin-2 (Uniprot code sp|Q99541|PLIN2_HUMAN).

FIG. 14 illustrates peptides corresponding to amino acid residues 1223-1255 of mucin-1 (Uniprot code sp|P15941|MUC1_HUMAN).

FIG. 15 illustrates peptides corresponding to amino acid residues 79-109 and 172-180 of kappa-casein (Uniprot code sp|P07498|CASK_HUMAN).

### DETAILED DESCRIPTION

1. Introduction

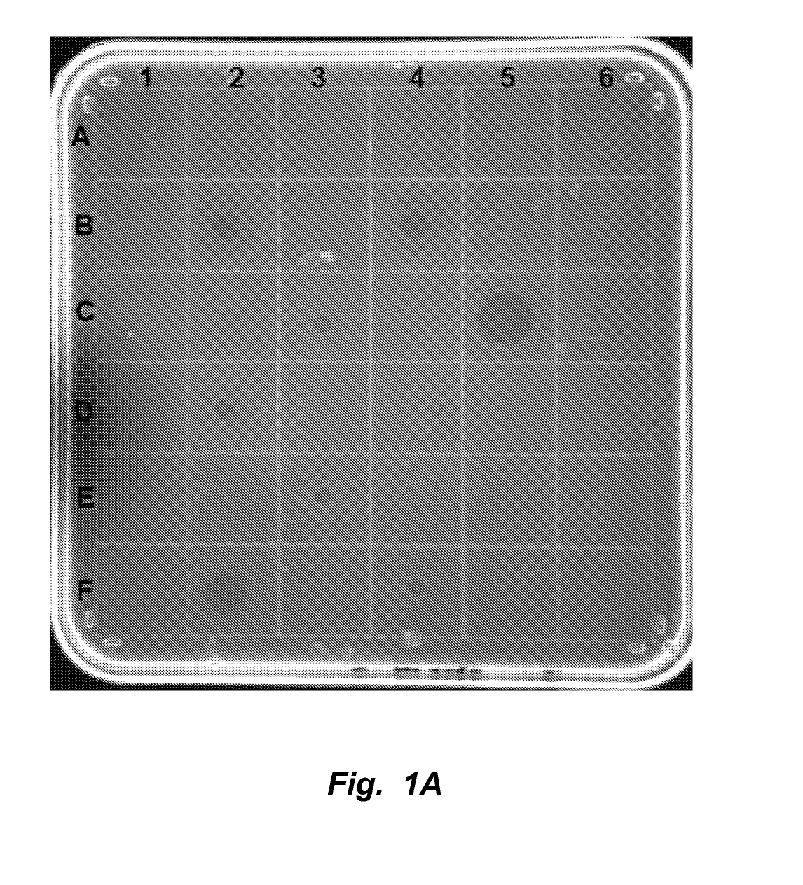

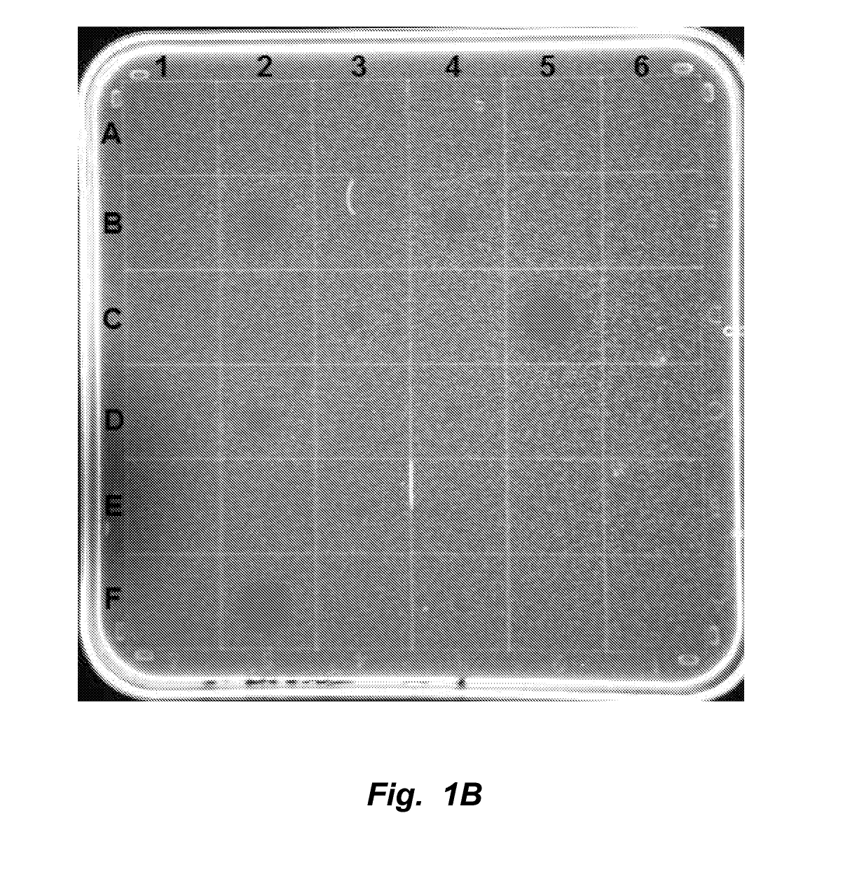

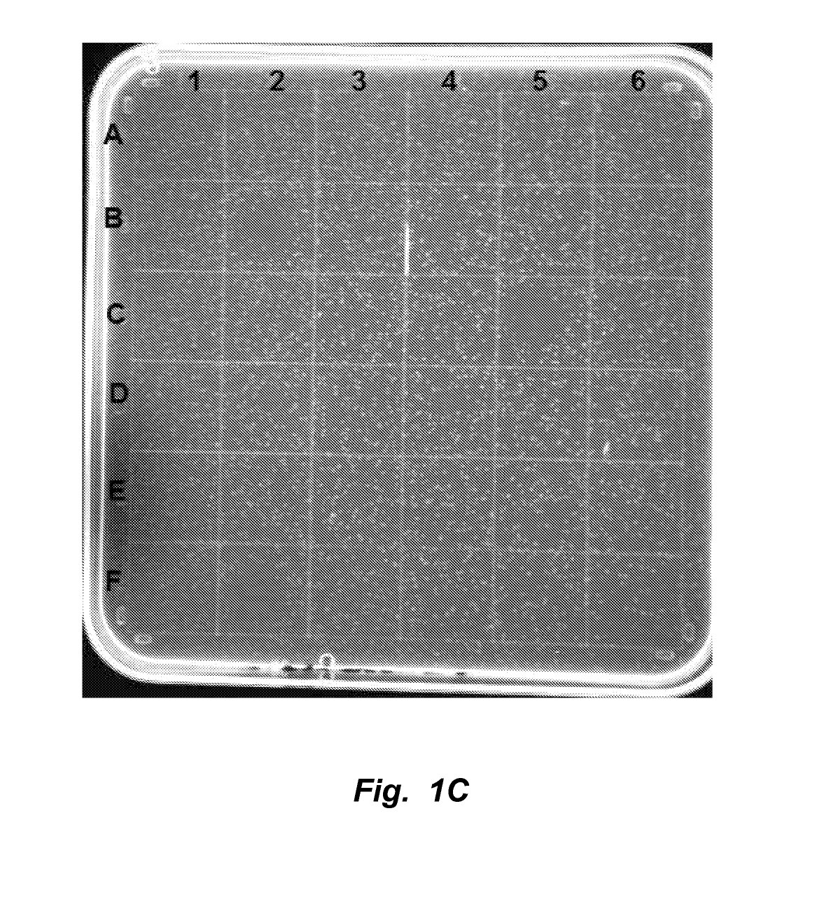

The present invention is based, in part, on the discovery of peptides in mammalian milk (e.g., human and bovine milk) with antibacterial activities. Peptides originally identified in mammalian milk have antibacterial functions. Antibacterial activity was shown against Escherichia coli and Staphylococcus aureus with microbial assays, as shown in Examples 1 and 2, and in FIGS. 1A-C and 2A-C. Mammalian milk peptides act in vivo to protect the nursing mother from infections including mastitis, a painful inflammation of the breast often caused by pathogenic bacteria such as S. aureus and E. coli.

Peptides were isolated from human milk by lipid removal by centrifugation, acid precipitation of proteins and oligosaccharide and salt removal via preparative reverse-phase chromatography. Peptides were then identified via nano-liquid-chromatography chip quadrupole time-of-flight tandem mass spectrometry (nanoLC-chip-Q-TOF).

Mammalian milk peptides, as well as homologs, analogs and mimetics thereof, find use to ameliorate and/or prevent bacterial infections, including epithelial and skin infections, infections of the oral cavity, and infections of the mammary gland. The peptides also can be used as a dietary supplement for normal and/or immunocompromised individuals. The peptides may also be used in combination with or in the place of chemical antibiotics, especially in the case of drug-resistant pathogens.

As a measure for preventing, reducing and/or treating various infections of epithelial surfaces, the described peptides are advantageous over traditional anti-microbial components, due to their inherent safety, unique selectivity and potential to complement other anti-microbial strategies. The safety is the result of their origin, they are secreted into mother's milk and in contrast to other anti-microbial components that disrupt other endogenous microbial ecosystems including the intestinal microbiome, peptides from milk do not adversely affect the development of a stable, protective gut flora, e.g., in an infant. Their efficacy is similarly the result of the evolution of lactation in the face of the threats to mammary tissue specifically. Because these peptides are present in mammalian milk their efficacy can complement other pharmaceuticals, including microbial and plant-derived pharmaceuticals.

2. Antibacterial Peptides

Peptides originating from, derived from, and/or purified or isolated from mammalian milk, and analogs thereof, which have antibacterial properties are provided (see, Table 1 (e.g., SEQ ID NOs:1-535) or Table 3, below). Generally, the peptides are subsequences of one or more mammalian milk proteins, including without limitation, e.g., polymeric immunoglobulin receptor (PIGR); beta-casein (CASB); alpha-S1-casein (CASA1); butyrophilin subfamily 1 member A1 (BT1A1); osteopontin (OSTP); mucin-1 (MUC1); perilipin-2 (PLIN2); neural Wiskott-Aldrich syndrome protein (WASL); cancer susceptibility candidate gene 3 protein (CASC3); inositol polyphosphate phosphatase-like 1 (SHIP2); protein diaphanous homolog 1 (DIAP1); ceruloplasmin (CERU); haptoglobin (HPT); complement C3 (CO3); pro-epidermal growth factor (EGF); protein disulfide-isomerase (PDIA1); kappa-casein (CASK); thrombospondin-1 (TSP1); heat shock protein HSP 90-beta (HS90B); complement C4-A (CO4A); receptor-type tyrosine-protein phosphatase alpha (PTPRA); bile salt-activated lipase (CEL); lactoperoxidase (PERL); macrophage mannose receptor 1 (MRC1); tenascin (TENA); xanthine dehydrogenase/oxidase (XDH); paxillin (PAXI); fatty acid synthase (FAS); centromere protein F (CENPF); afadin (AFAD); heterogeneous nuclear ribonucleoprotein K (HNRPK); disks large homolog 4 (DLG4); arginase-2, mitochondrial (ARGI2); tyrosine-protein phosphatase non-receptor type 13 (PTN13); E3 ubiquitin-protein ligase CBL-B (CBLB); protein scribble homolog (SCRIB); dedicator of cytokinesis protein 1 (DOCK1); telomeric repeat-binding factor 2 (TERF2); inverted formin-2 (INF2); programmed cell death protein 4 (PDCD4); E3 ubiquitin-protein ligase UBR4 (UBR4); NMDA receptor-regulated protein 2 (NARG2); 1a-related protein 1 (LARP1); prostate androgen-regulated mucin-like protein 1 (PARM1); ubiquitin carboxyl-terminal hydrolase 51 (UBP51); chromatin complexes subunit BAP18 (BAP18); Armadillo repeat-containing protein 10 (ARM10); misshapen-like kinase 1 (MINK1); protein enabled homolog (ENAH); biorientation of chromosomes in cell division protein 1-like 1 (BD1L1); short transient receptor potential channel 4-associated protein (TP4AP); ankyrin repeat and SAM domain-containing protein 1A (ANS1A); mitogen-activated protein kinase kinase kinase kinase 1 (M4K1); GDP-fucose transporter 1 (FUCT1); E3 ubiquitin-protein ligase UHRF1 (UHRF1); mucin-4 (MUC-4); matrix metalloproteinase-19 (MMP19); serine/threonine-protein kinase 33 (STK33); TR10 and F-actin-binding protein (TARA); apoptotic chromatin condensation inducer in the nucleus (ACINU); UPF0760 protein C2orf29 (CB029); zinc finger protein PLAGL1 (PLAL1); cofilin-2 (COF2); sialic acid-binding Ig-like lectin 9 (SIGL9); protein VPRBP (VPRBP); myosin-4 (MYH4); endoplasmic reticulum mannosyl-oligosaccharide 1,2-alpha-mannosidase (MAN1B1); and cDNA F1157167, highly similar to Etoposide-induced protein 2.4.

Effective amounts of the peptides can reduce, inhibit, delay and/or prevent the growth or proliferation of a bacterial organism (e.g., E. coli and/or S. aureus). In varying embodiments, the individual peptides are generally about 5 to about 55 amino acid residues in length, e.g., about 6 amino acids to about 50 amino acids residues in length. In varying embodiments, the individual peptides are no longer than 60 amino acids in length, e.g., no longer than 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6 or 5 amino acids in length. In varying embodiments, the peptides have from about 5 to about 55 amino acid residues, e.g., from about 6 to about 50 amino acid residues, from about 7 to about 45 amino acid residues, from about 8 to about 40 amino acid residues, from about 9 to about 35 amino acid residues. In some embodiments, the isolated and/or purified peptides have a molecular weight less than 15 kDa, e.g., less than about 10 kDa, 9 kDa, 8 kDa, 7 kDa or 6 kDa, e.g., in the range of about 0.4 kDa to about 5.8 kDa, e.g., about 0.5-5.0 kDa, about 0.6-4.5 kDa, about 0.7-4.0 kDa, about 0.8-3.5 kDa, e.g., have a molecular weight that is at least about 0.4 kDa, 0.5 kDa, 0.6 kDa, 0.7 kDa, 0.8 kDa and up to about 3.5 kDa, 4.0 kDa, 4.5 kDa, 5.0 kDa, 5.5 kDa or about 5.8 kDa.

In some embodiments, the peptide comprises one or more modifications selected from the group consisting of:

i) oxidation or dioxidation of one or more methionine (M) residues;

ii) deamination of one or more glutamine (Q) residues; and/or

iii) phosphorylation of one or more serine (S), threonine (T) or tyrosine (Y) residues.

In some embodiments, the peptide comprises one or more modifications selected from the group consisting of:

i) one or more of the amino acid residues are D-amino acids, for example, 2, 3, 4, 5, 6, 7, 8, 9, 10, 15, 20, 25, 30, 35, 40, 45, 50, 55 or all, of the amino acid residues are D-amino acids;

ii) the peptide comprises protecting groups at one or both of the N-terminus or the C terminus; iii) the peptide is fully or partially retro-inverso; and/or

iv) the peptide is circularized.

In varying embodiments, the peptide comprises 1 or more substituted, added or deleted amino acid residues, e.g., 2, 3, 4, 5, 6, 7, 8, 9, or 10 substituted, added or deleted amino acid residues. In varying embodiments, the peptide comprises 1 or more substituted, added or deleted amino acid residues such that the peptide has at least 60% amino acid sequence identity, e.g., at least 65%, 70%, 75%, 80%, 85%, 90%, 95%, 96%, 97%, 98%, or 99% amino acid sequence identity to a peptide of Table 1, e.g., a peptide of SEQ ID NOs: 1-535 or a peptide of Table 3.

In some embodiments, the peptides may have from 1 to 5 flanking L- or D-cysteine residues at the N-terminal and C-terminal ends, e.g., to allow for circularization and/or conjugation of the peptide. In some embodiments, cysteine residues can be added to the amino and carboxy terminus to allow for circularization. In varying embodiments, additional amino acid residues (e.g., X is any amino acid residue) can be added to either the amino and/or carboxyl terminus, for example, from 1-5 amino acid residues, for example, 1, 2, 3, 4 or 5 amino acid residues to either the amino and/or carboxyl terminus.

In some embodiments, the peptide comprises 2 or more repeats, for example, 3, 4, 5, 6 or more repeats. The repeats can be tandem, directly linked or linked via a spacer sequence (e.g., a flexible linker sequence, e.g., GGGGS).

In varying embodiments, one or more of the peptides of Table 1 (e.g., SEQ ID NOs: 1-535) or Table 3 are comprised in a polypeptide, e.g., as a fusion protein. The polypeptides can comprise antibacterial peptides, described herein, operably linked with heterologous amino acid sequences. In varying embodiments, the polypeptides comprise two or more antibacterial peptides, described herein. In some embodiments, the polypeptide is no longer than 300 amino acids in length, for example, no longer than 250, 200, 150, 100, 75, 50 or 25 amino acids in length. The peptides in a polypeptide can be tandem, directly linked or linked via a spacer sequence (e.g., a flexible linker sequence, e.g., GGGGS).

TABLE 1
Antibacterial Peptides Identified in Skim Human Milk at 95% Confidence Level

| SEQ ID | | Protein of origin | |
|---|---|---|---|
| NO: | Sequence | (uniprot code) | Protein Name |
| 1 | PPSRPSVAVPPPPP | sp\|O00401\|WASL_HUMAN | neural Wiskott- |
| | | | Aldrich syndrome |
| | | | protein |
| 2 | RQAPPPPPP | sp\|O00401\|WASL_HUMAN | neural Wiskott- |
| | | | Aldrich syndrome |
| | | | protein |
| 3 | SRPSVAVPPPPP | sp\|O00401\|WASL_HUMAN | neural Wiskott- |
| | | | Aldrich syndrome |
| | | | protein |
| 4 | PSPEADAPVLGSPEKEEAASEPPAAAPDA | sp\|O15234\|CASC3_HUMAN | cancer susceptibility |
| | | | candidate gene 3 |
| | | | protein |
| 5 | KTLDEVTVTIPHDI | sp\|O15357\|SHIP2_HUMAN | inositol |
| | | | polyphosphate |
| | | | phosphatase-like 1 |
| 6 | SLPGGTAIPPPPP | sp\|O60610\|DIAP1_HUMAN | protein diaphanous |
| | | | homolog 1 |
| 7 | GSYKKLVYRE | sp\|P00450\|CERU_HUMAN | ceruloplasmin |
| 8 | AGSAFA | sp\|P00738\|HPT_HUMAN | haptoglobin |
| 9 | ITHRIHWESAS | sp\|P01024\|CO3_HUMAN | complement C3 |
| 10 | VLYRIFTVN | sp\|P01024\|CO3_HUMAN | complement C3 |
| 11 | KLLSKNPKNPYEESSR | sp\|P01133\|EGF_HUMAN | pro-epidermal growth |
| | | | factor |
| 12 | AAPDEKVLDSGFREIENK | sp\|P01833\|PIGR_HUMAN | polymeric |
| | | | immunoglobulin |
| | | | receptor |
| 13 | ADAAPDEKVLDSGFREIENK | sp\|P01833\|PIGR_HUMAN | polymeric |
| | | | immunoglobulin |
| | | | receptor |
| 14 | ADTRDQADGSRASVDSGSSEEQGGSSRA | sp\|P01833\|PIGR_HUMAN | polymeric |
| | | | immunoglobulin |
| | | | receptor |
| 15 | AEEKAVADTRDQADGSR | sp\|P01833\|PIGR_HUMAN | polymeric |
| | | | immunoglobulin |
| | | | receptor |
| 16 | AIQDPRLFAEEKAVADTR | sp\|P01833\|PIGR_HUMAN | polymeric |
| | | | immunoglobulin |
| | | | receptor |
| 17 | AIQDPRLFAEEKAVADTRDQADGS | sp\|P01833\|PIGR_HUMAN | polymeric |
| | | | immunoglobulin |
| | | | receptor |
| 18 | ASVDSGSSEEQGGSSRALVSTLVP | sp\|P01833\|PIGR_HUMAN | polymeric |
| | | | immunoglobulin |
| | | | receptor |
| 19 | ASVDSGSSEEQGGSSRALVSTLVPLG | sp\|P01833\|PIGR_HUMAN | polymeric |
| | | | immunoglobulin |
| | | | receptor |
| 20 | AVADTRDQAD | sp\|P01833\|PIGR_HUMAN | polymeric |
| | | | immunoglobulin |
| | | | receptor |
| 21 | AVADTRDQADG | sp\|P01833\|PIGR_HUMAN | polymeric |
| | | | immunoglobulin |
| | | | receptor |
| 22 | AVADTRDQADGS | sp\|P01833\|PIGR_HUMAN | polymeric |
| | | | immunoglobulin |
| | | | receptor |
| 23 | AVADTRDQADGSRAS | sp\|P01833\|PIGR_HUMAN | polymeric |
| | | | immunoglobulin |
| | | | receptor |
| 24 | AVADTRDQADGSRASVD | sp\|P01833\|PIGR_HUMAN | polymeric |
| | | | immunoglobulin |
| | | | receptor |
| 25 | AVADTRDQADGSRASVDSG | sp\|P01833\|PIGR_HUMAN | polymeric |
| | | | immunoglobulin |
| | | | receptor |
| 26 | AVADTRDQADGSRASVDSGSSEEQG | sp\|P01833\|PIGR_HUMAN | polymeric |
| | | | immunoglobulin |
| | | | receptor |
| 27 | AVADTRDQADGSRASVDSGSSEEQGG | sp\|P01833\|PIGR_HUMAN | polymeric |
| | | | immunoglobulin |
| | | | receptor |
| 28 | AVADTRDQADGSRASVDSGSSEEQGGSS | sp\|P01833\|PIGR_HUMAN | polymeric |
| | | | immunoglobulin |
| | | | receptor |
| 29 | AVADTRDQADGSRASVDSGSSEEQGGSSRA | sp\|P01833\|PIGR_HUMAN | polymeric |
| | | | immunoglobulin |
| | | | receptor |
| 30 | AVADTRDQADGSRASVDSGSSEEQGGSSRAL | sp\|P01833\|PIGR_HUMAN | polymeric |
| | | | immunoglobulin |
| | | | receptor |
| 31 | AVADTRDQADGSRASVDSGSSEEQGGSSRALVST | sp\|P01833\|PIGR_HUMAN | polymeric |
| | | | immunoglobulin |
| | | | receptor |
| 32 | AVADTRDQADGSRASVDSGSSEEQGGSSRALVSTLVP | sp\|P01833\|PIGR_HUMAN | polymeric |
| | | | immunoglobulin |
| | | | receptor |
| 33 | AVADTRDQADGSRASVDSGSSEEQGGSSRALVSTLVPLG | sp\|P01833\|PIGR_HUMAN | polymeric |
| | | | immunoglobulin |
| | | | receptor |
| 34 | DAAPDEKVLDSGFREIENK | sp\|P01833\|PIGR_HUMAN | polymeric |
| | | | immunoglobulin |
| | | | receptor |
| 35 | DGSRASVDSGSSEEQGGSSR | sp\|P01833\|PIGR_HUMAN | polymeric |
| | | | immunoglobulin |
| | | | receptor |
| 36 | DGSRASVDSGSSEEQGGSSRA | sp\|P01833\|PIGR_HUMAN | polymeric |
| | | | immunoglobulin |
| | | | receptor |
| 37 | DPRLFAEEKAVADTR | sp\|P01833\|PIGR_HUMAN | polymeric |
| | | | immunoglobulin |
| | | | receptor |
| 38 | DQADGSRASVDSGSSEEQGGSS | sp\|P01833\|PIGR_HUMAN | polymeric |
| | | | immunoglobulin |
| | | | receptor |
| 39 | DQADGSRASVDSGSSEEQGGSSRA | sp\|P01833\|PIGR_HUMAN | polymeric |
| | | | immunoglobulin |
| | | | receptor |
| 40 | DQADGSRASVDSGSSEEQGGSSRAL | sp\|P01833\|PIGR_HUMAN | polymeric |
| | | | immunoglobulin |
| | | | receptor |
| 41 | DQADGSRASVDSGSSEEQGGSSRALVS | sp\|P01833\|PIGR_HUMAN | polymeric |
| | | | immunoglobulin |
| | | | receptor |
| 42 | DQADGSRASVDSGSSEEQGGSSRALVST | sp\|P01833\|PIGR_HUMAN | polymeric |
| | | | immunoglobulin |
| | | | receptor |
| 43 | DQADGSRASVDSGSSEEQGGSSRALVSTLVP | sp\|P01833\|PIGR_HUMAN | polymeric |
| | | | immunoglobulin |
| | | | receptor |
| 44 | DQADGSRASVDSGSSEEQGGSSRALVSTLVPL | sp\|P01833\|PIGR_HUMAN | polymeric |
| | | | immunoglobulin |
| | | | receptor |
| 45 | DQADGSRASVDSGSSEEQGGSSRALVSTLVPLG | sp\|P01833\|PIGR_HUMAN | polymeric |
| | | | immunoglobulin |
| | | | receptor |
| 46 | DSGSSEEQGGSSRAL | sp\|P01833\|PIGR_HUMAN | polymeric |
| | | | immunoglobulin |
| | | | receptor |
| 47 | DSGSSEEQGGSSRALV | sp\|P01833\|PIGR_HUMAN | polymeric |
| | | | immunoglobulin |
| | | | receptor |
| 48 | DSGSSEEQGGSSRALVST | sp\|P01833\|PIGR_HUMAN | polymeric |
| | | | immunoglobulin |
| | | | receptor |
| 49 | DSGSSEEQGGSSRALVSTLVP | sp\|P01833\|PIGR_HUMAN | polymeric |
| | | | immunoglobulin |
| | | | receptor |
| 50 | DSGSSEEQGGSSRALVSTLVPL | sp\|P01833\|PIGR_HUMAN | polymeric |
| | | | immunoglobulin |
| | | | receptor |
| 51 | DSGSSEEQGGSSRALVSTLVPLG | sp\|P01833\|PIGR_HUMAN | polymeric |
| | | | immunoglobulin |
| | | | receptor |
| 52 | EEKAVADTRDQADG | sp\|P01833\|PIGR_HUMAN | polymeric |
| | | | immunoglobulin |
| | | | receptor |
| 53 | EEKAVADTRDQADGSR | sp\|P01833\|PIGR_HUMAN | polymeric |
| | | | immunoglobulin |
| | | | receptor |
| 54 | EKAVADTRDQADG | sp\|P01833\|PIGR_HUMAN | polymeric |
| | | | immunoglobulin |
| | | | receptor |
| 55 | FAEEKAVADTRDQADGSR | sp\|P01833\|PIGR_HUMAN | polymeric |
| | | | immunoglobulin |
| | | | receptor |
| 56 | FAEEKAVADTRDQADGSRASVDSGSSEEQGGSSRA | sp\|P01833\|PIGR_HUMAN | polymeric |
| | | | immunoglobulin |
| | | | receptor |
| 57 | KADAAPDEKVLDSGFREIENK | sp\|P01833\|PIGR_HUMAN | polymeric |
| | | | immunoglobulin |
| | | | receptor |
| 58 | LFAEEKAVADTRDQADGSR | sp\|P01833\|PIGR_HUMAN | polymeric |
| | | | immunoglobulin |
| | | | receptor |
| 59 | LFAEEKAVADTRDQADGSRASVDSGSSEEQGGSSRA | sp\|P01833\|PIGR_HUMAN | polymeric |
| | | | immunoglobulin |
| | | | receptor |
| 60 | QADGSRASVDSGSSEEQGGSSRA | sp\|P01833\|PIGR_HUMAN | polymeric |
| | | | immunoglobulin |
| | | | receptor |
| 61 | SVDSGSSEEQGGSSRA | sp\|P01833\|PIGR_HUMAN | polymeric |
| | | | immunoglobulin |
| | | | receptor |
| 62 | SVDSGSSEEQGGSSRALVST | sp\|P01833\|PIGR_HUMAN | polymeric |
| | | | immunoglobulin |
| | | | receptor |
| 63 | SVDSGSSEEQGGSSRALVSTLVP | sp\|P01833\|PIGR_HUMAN | polymeric |
| | | | immunoglobulin |
| | | | receptor |
| 64 | SVDSGSSEEQGGSSRALVSTLVPL | sp\|P01833\|PIGR_HUMAN | polymeric |
| | | | immunoglobulin |
| | | | receptor |
| 65 | SVDSGSSEEQGGSSRALVSTLVPLG | sp\|P01833\|PIGR_HUMAN | polymeric |
| | | | immunoglobulin |
| | | | receptor |
| 66 | TRDQADGSRASVDSGSSEEQGGSSRA | sp\|P01833\|PIGR_HUMAN | polymeric |
| | | | immunoglobulin |
| | | | receptor |
| 67 | VADTRDQADGSRAS | sp\|P01833\|PIGR_HUMAN | polymeric |
| | | | immunoglobulin |
| | | | receptor |
| 68 | VADTRDQADGSRASVDSGSSEEQGGSS | sp\|P01833\|PIGR_HUMAN | polymeric |
| | | | immunoglobulin |
| | | | receptor |
| 69 | VADTRDQADGSRASVDSGSSEEQGGSSRA | sp\|P01833\|PIGR_HUMAN | polymeric |
| | | | immunoglobulin |
| | | | receptor |
| 70 | VDSGSSEEQGGSSRA | sp\|P01833\|PIGR_HUMAN | polymeric |
| | | | immunoglobulin |
| | | | receptor |
| 71 | VDSGSSEEQGGSSRALVSTLVP | sp\|P01833\|PIGR_HUMAN | polymeric |
| | | | immunoglobulin |
| | | | receptor |
| 72 | VDSGSSEEQGGSSRALVSTLVPLG | sp\|P01833\|PIGR_HUMAN | polymeric |
| | | | immunoglobulin |
| | | | receptor |
| 73 | YGETAAVYVAVEERKAAGSR | sp\|P01833\|PIGR_HUMAN | polymeric |
| | | | immunoglobulin |
| | | | receptor |
| 512 | DQADGSRASVDSGSSEEQGGSSR | sp\|P01833\|PIGR_HUMAN | polymeric |
| | | | immunoglobulin |
| | | | receptor |
| 513 | GSSEEQGGSSRALV | sp\|P01833\|PIGR_HUMAN | polymeric |
| | | | immunoglobulin |
| | | | receptor |
| 74 | AKDTVYTKGRVMPVLK | sp\|P05814\|CASB_HUMAN | beta-casein |
| 75 | ALLLNQELLLNPTHQIYPVT | sp\|P05814\|CASB_HUMAN | beta-casein |
| 76 | ALLLNQELLLNPTHQIYPVTQPLAPVHNPISV | sp\|P05814\|CASB_HUMAN | beta-casein |
| 77 | APVHNPISV | sp\|P05814\|CASB_HUMAN | beta-casein |
| 78 | AQPAVVLPVPQPEIMEVPK | sp\|P05814\|CASB_HUMAN | beta-casein |
| 79 | AQPAVVLPVPQPEIMEVPKAKDTVYT | sp\|P05814\|CASB_HUMAN | beta-casein |
| 80 | AQPAVVLPVPQPEIMEVPKAKDTVYTK | sp\|P05814\|CASB_HUMAN | beta-casein |
| 81 | AQPAVVLPVPQPEIMEVPKAKDTVYTKG | sp\|P05814\|CASB_HUMAN | beta-casein |
| 82 | AVPVQALLLNQELLLNPTHQIYPVTQPLAPVHNPISV | sp\|P05814\|CASB_HUMAN | beta-casein |
| 83 | DEHQDKI | sp\|P05814\|CASB_HUMAN | beta-casein |
| 84 | DEHQDKIYP | sp\|P05814\|CASB_HUMAN | beta-casein |
| 85 | DLENLHLP | sp\|P05814\|CASB_HUMAN | beta-casein |
| 86 | DLENLHLPLP | sp\|P05814\|CASB_HUMAN | beta-casein |
| 87 | DPQIPKL | sp\|P05814\|CASB_HUMAN | beta-casein |
| 88 | DPQIPKLTDLE | sp\|P05814\|CASB_HUMAN | beta-casein |
| 89 | DPQIPKLTDLENLHLPLP | sp\|P05814\|CASB_HUMAN | beta-casein |
| 90 | DTVYTKGR | sp\|P05814\|CASB_HUMAN | beta-casein |
| 91 | DTVYTKGRV | sp\|P05814\|CASB_HUMAN | beta-casein |
| 92 | DTVYTKGRVMPVL | sp\|P05814\|CASB_HUMAN | beta-casein |
| 93 | DTVYTKGRVMPVLK | sp\|P05814\|CASB_HUMAN | beta-casein |
| 94 | EESITEYK | sp\|P05814\|CASB_HUMAN | beta-casein |
| 95 | EIMEVPK | sp\|P05814\|CASB_HUMAN | beta-casein |
| 96 | EIMEVPKAKDTVYT | sp\|P05814\|CASB_HUMAN | beta-casein |
| 97 | EKVKHEDQQQGEDEHQDK | sp\|P05814\|CASB_HUMAN | beta-casein |
| 98 | ELLLNPTHQIYP | sp\|P05814\|CASB_HUMAN | beta-casein |
| 99 | ELLLNPTHQIYPVT | sp\|P05814\|CASB_HUMAN | beta-casein |
| 100 | ELLLNPTHQIYPVTQ | sp\|P05814\|CASB_HUMAN | beta-casein |
| 101 | ELLLNPTHQIYPVTQPLAPVHNPISV | sp\|P05814\|CASB_HUMAN | beta-casein |
| 102 | ENLHLPLPLL | sp\|P05814\|CASB_HUMAN | beta-casein |
| 103 | ENLHLPLPLLQ | sp\|P05814\|CASB_HUMAN | beta-casein |
| 104 | ESITEYK | sp\|P05814\|CASB_HUMAN | beta-casein |
| 105 | ESLSSSEESITE | sp\|P05814\|CASB_HUMAN | beta-casein |
| 106 | ESLSSSEESITEYK | sp\|P05814\|CASB_HUMAN | beta-casein |
| 107 | ETIESLSSSEE | sp\|P05814\|CASB_HUMAN | beta-casein |
| 108 | ETIESLSSSEESITE | sp\|P05814\|CASB_HUMAN | beta-casein |
| 109 | ETIESLSSSEESITEY | sp\|P05814\|CASB_HUMAN | beta-casein |
| 110 | ETIESLSSSEESITEYK | sp\|P05814\|CASB_HUMAN | beta-casein |
| 111 | ETIESLSSSEESITEYKQ | sp\|P05814\|CASB_HUMAN | beta-casein |
| 112 | ETIESLSSSEESITEYKQK | sp\|P05814\|CASB_HUMAN | beta-casein |
| 113 | ETIESLSSSEESITEYKQKVEK | sp\|P05814\|CASB_HUMAN | beta-casein |
| 114 | EVPKAKDT | sp\|P05814\|CASB_HUMAN | beta-casein |
| 115 | EVPKAKDTVYT | sp\|P05814\|CASB_HUMAN | beta-casein |
| 116 | EVPKAKDTVYTK | sp\|P05814\|CASB_HUMAN | beta-casein |
| 117 | FDPQIPK | sp\|P05814\|CASB_HUMAN | beta-casein |
| 118 | FDPQIPKL | sp\|P05814\|CASB_HUMAN | beta-casein |
| 119 | FDPQIPKLT | sp\|P05814\|CASB_HUMAN | beta-casein |
| 120 | FDPQIPKLTD | sp\|P05814\|CASB_HUMAN | beta-casein |
| 121 | FFDPQIPK | sp\|P05814\|CASB_HUMAN | beta-casein |
| 122 | GEDEHQDK | sp\|P05814\|CASB_HUMAN | beta-casein |
| 123 | GEDEHQDKIYPS | sp\|P05814\|CASB_HUMAN | beta-casein |
| 124 | GRVMPVLK | sp\|P05814\|CASB_HUMAN | beta-casein |
| 125 | GRVMPVLKSPT | sp\|P05814\|CASB_HUMAN | beta-casein |
| 126 | GRVMPVLKSPTIP | sp\|P05814\|CASB_HUMAN | beta-casein |
| 127 | GRVMPVLKSPTIPFFDPQIPK | sp\|P05814\|CASB_HUMAN | beta-casein |
| 128 | GRVMPVLKSPTIPFFDPQIPKLTD | sp\|P05814\|CASB_HUMAN | beta-casein |
| 129 | HEDQQQGEDEHQDK | sp\|P05814\|CASB_HUMAN | beta-casein |
| 130 | HEDQQQGEDEHQDKIYP | sp\|P05814\|CASB_HUMAN | beta-casein |
| 131 | HEDQQQGEDEHQDKIYPS | sp\|P05814\|CASB_HUMAN | beta-casein |
| 132 | HLPLPLL | sp\|P05814\|CASB_HUMAN | beta-casein |
| 133 | HNPISV | sp\|P05814\|CASB_HUMAN | beta-casein |
| 134 | HQIYPVTQPLAPVHNPISV | sp\|P05814\|CASB_HUMAN | beta-casein |
| 135 | IESLSSSEESITEYK | sp\|P05814\|CASB_HUMAN | beta-casein |
| 136 | IPQQVVPYPQRAVPVQA | sp\|P05814\|CASB_HUMAN | beta-casein |
| 137 | IYPVTQPLAPVHNPISV | sp\|P05814\|CASB_HUMAN | beta-casein |
| 138 | KDTVYTKGRVMPVL | sp\|P05814\|CASB_HUMAN | beta-casein |
| 139 | KDTVYTKGRVMPVLK | sp\|P05814\|CASB_HUMAN | beta-casein |
| 140 | KHEDQQQGEDEHQD | sp\|P05814\|CASB_HUMAN | beta-casein |
| 141 | KVEKVKHEDQQQG | sp\|P05814\|CASB_HUMAN | beta-casein |
| 142 | KVEKVKHEDQQQGEDEHQDK | sp\|P05814\|CASB_HUMAN | beta-casein |
| 143 | LENLHLPLP | sp\|P05814\|CASB_HUMAN | beta-casein |
| 144 | LENLHLPLPLLQ | sp\|P05814\|CASB_HUMAN | beta-casein |
| 145 | LLLNPTHQIYP | sp\|P05814\|CASB_HUMAN | beta-casein |
| 146 | LLLNPTHQIYPVT | sp\|P05814\|CASB_HUMAN | beta-casein |
| 147 | LLLNPTHQIYPVTQ | sp\|P05814\|CASB_HUMAN | beta-casein |
| 148 | LLLNPTHQIYPVTQPLAP | sp\|P05814\|CASB_HUMAN | beta-casein |
| 149 | LLLNPTHQIYPVTQPLAPVH | sp\|P05814\|CASB_HUMAN | beta-casein |
| 150 | LLLNPTHQIYPVTQPLAPVHNPISV | sp\|P05814\|CASB_HUMAN | beta-casein |
| 151 | LLLNQELLLNPTHQIYPVTQ | sp\|P05814\|CASB_HUMAN | beta-casein |
| 152 | LLLNQELLLNPTHQIYPVTQPLAPVHNPISV | sp\|P05814\|CASB_HUMAN | beta-casein |
| 153 | LLNPTHQIYP | sp\|P05814\|CASB_HUMAN | beta-casein |
| 154 | LLNPTHQIYPVTQPLAPVH | sp\|P05814\|CASB_HUMAN | beta-casein |
| 155 | LLNPTHQIYPVTQPLAPVHNPIS | sp\|P05814\|CASB_HUMAN | beta-casein |
| 156 | LLNPTHQIYPVTQPLAPVHNPISV | sp\|P05814\|CASB_HUMAN | beta-casein |
| 157 | LLNQELLLNPTHQ | sp\|P05814\|CASB_HUMAN | beta-casein |
| 158 | LLNQELLLNPTHQIYPVT | sp\|P05814\|CASB_HUMAN | beta-casein |
| 159 | LLNQELLLNPTHQIYPVTQ | sp\|P05814\|CASB_HUMAN | beta-casein |
| 160 | LLNQELLLNPTHQIYPVTQPLAPVHNPISV | sp\|P05814\|CASB_HUMAN | beta-casein |
| 161 | LLQPLMQQVPQPIPQT | sp\|P05814\|CASB_HUMAN | beta-casein |
| 162 | LLQPLMQQVPQPIPQTL | sp\|P05814\|CASB_HUMAN | beta-casein |
| 163 | LMQQVPQPIPQT | sp\|P05814\|CASB_HUMAN | beta-casein |
| 164 | LNPTHQIYPVTQ | sp\|P05814\|CASB_HUMAN | beta-casein |
| 165 | LNPTHQIYPVTQPLAPVHNPISV | sp\|P05814\|CASB_HUMAN | beta-casein |
| 166 | LNQELLLNPT | sp\|P05814\|CASB_HUMAN | beta-casein |
| 167 | LNQELLLNPTHQ | sp\|P05814\|CASB_HUMAN | beta-casein |
| 168 | LNQELLLNPTHQIYPVT | sp\|P05814\|CASB_HUMAN | beta-casein |
| 169 | LNQELLLNPTHQIYPVTQPLAPVHNPISV | sp\|P05814\|CASB_HUMAN | beta-casein |
| 170 | LPIPQQVVPYP | sp\|P05814\|CASB_HUMAN | beta-casein |
| 171 | LPIPQQVVPYPQRAVP | sp\|P05814\|CASB_HUMAN | beta-casein |
| 172 | LPIPQQVVPYPQRAVPVQ | sp\|P05814\|CASB_HUMAN | beta-casein |
| 173 | LPIPQQVVPYPQRAVPVQA | sp\|P05814\|CASB_HUMAN | beta-casein |
| 174 | LPVPQPEI | sp\|P05814\|CASB_HUMAN | beta-casein |
| 175 | LPVPQPEIM | sp\|P05814\|CASB_HUMAN | beta-casein |
| 176 | LPVPQPEIME | sp\|P05814\|CASB_HUMAN | beta-casein |
| 177 | LPVPQPEIMEVPK | sp\|P05814\|CASB_HUMAN | beta-casein |
| 178 | LSSSEESITEYK | sp\|P05814\|CASB_HUMAN | beta-casein |
| 179 | LSSSEESITEYKQKVEK | sp\|P05814\|CASB_HUMAN | beta-casein |
| 180 | MEVPKAKDTVYTKGR | sp\|P05814\|CASB_HUMAN | beta-casein |
| 181 | NILPLAQPAVVLPVPQPEIMEVPK | sp\|P05814\|CASB_HUMAN | beta-casein |
| 182 | NLHLPLP | sp\|P05814\|CASB_HUMAN | beta-casein |
| 183 | NPTHQIYPVTQ | sp\|P05814\|CASB_HUMAN | beta-casein |
| 184 | NPTHQIYPVTQPLAPVHNPISV | sp\|P05814\|CASB_HUMAN | beta-casein |
| 185 | NQELLLNPT | sp\|P05814\|CASB_HUMAN | beta-casein |
| 186 | NQELLLNPTHQIYP | sp\|P05814\|CASB_HUMAN | beta-casein |
| 187 | NQELLLNPTHQIYPVT | sp\|P05814\|CASB_HUMAN | beta-casein |
| 188 | NQELLLNPTHQIYPVTQ | sp\|P05814\|CASB_HUMAN | beta-casein |
| 189 | NQELLLNPTHQIYPVTQPLAPVH | sp\|P05814\|CASB_HUMAN | beta-casein |
| 190 | NQELLLNPTHQIYPVTQPLAPVHNPISV | sp\|P05814\|CASB_HUMAN | beta-casein |
| 191 | PAVVLPVPQPEI | sp\|P05814\|CASB_HUMAN | beta-casein |
| 192 | PAVVLPVPQPEIME | sp\|P05814\|CASB_HUMAN | beta-casein |
| 193 | PAVVLPVPQPEIMEVPKAK | sp\|P05814\|CASB_HUMAN | beta-casein |
| 194 | PAVVLPVPQPEIMEVPKAKDTVYTKGR | sp\|P05814\|CASB_HUMAN | beta-casein |
| 195 | PIPQQVVPYPQRAV | sp\|P05814\|CASB_HUMAN | beta-casein |
| 196 | PIPQQVVPYPQRAVPVQ | sp\|P05814\|CASB_HUMAN | beta-casein |
| 197 | PLAPVHNPISV | sp\|P05814\|CASB_HUMAN | beta-casein |
| 198 | PLAQPAVVLPVPQPEI | sp\|P05814\|CASB_HUMAN | beta-casein |
| 199 | PLMQQVPQPIPQTL | sp\|P05814\|CASB_HUMAN | beta-casein |
| 200 | PQIPKLTD | sp\|P05814\|CASB_HUMAN | beta-casein |
| 201 | PQIPKLTDLENL | sp\|P05814\|CASB_HUMAN | beta-casein |
| 202 | PTHQIYPVTQ | sp\|P05814\|CASB_HUMAN | beta-casein |
| 203 | PTHQIYPVTQPLAPVHNPISV | sp\|P05814\|CASB_HUMAN | beta-casein |
| 204 | PTIPFFDPQIPKLTD | sp\|P05814\|CASB_HUMAN | beta-casein |
| 205 | PVHNPISV | sp\|P05814\|CASB_HUMAN | beta-casein |
| 206 | PVPQPEI | sp\|P05814\|CASB_HUMAN | beta-casein |
| 207 | PVTQPLAPVHNPISV | sp\|P05814\|CASB_HUMAN | beta-casein |
| 208 | QELLLNPTHQIYP | sp\|P05814\|CASB_HUMAN | beta-casein |
| 209 | QELLLNPTHQIYPVT | sp\|P05814\|CASB_HUMAN | beta-casein |
| 210 | QELLLNPTHQIYPVTQPLAPVHNPISV | sp\|P05814\|CASB_HUMAN | beta-casein |
| 211 | QIYPVTQPLAPVHNPISV | sp\|P05814\|CASB_HUMAN | beta-casein |
| 212 | QKVEKVK | sp\|P05814\|CASB_HUMAN | beta-casein |
| 213 | QKVEKVKHED | sp\|P05814\|CASB_HUMAN | beta-casein |
| 214 | QKVEKVKHEDQQQGEDEHQD | sp\|P05814\|CASB_HUMAN | beta-casein |
| 215 | QKVEKVKHEDQQQGEDEHQDK | sp\|P05814\|CASB_HUMAN | beta-casein |
| 216 | QPAVVLPVPQPEI | sp\|P05814\|CASB_HUMAN | beta-casein |
| 217 | QPAVVLPVPQPEIM | sp\|P05814\|CASB_HUMAN | beta-casein |
| 218 | QPAVVLPVPQPEIMEVPK | sp\|P05814\|CASB_HUMAN | beta-casein |
| 219 | QPAVVLPVPQPEIMEVPKA | sp\|P05814\|CASB_HUMAN | beta-casein |
| 220 | QPAVVLPVPQPEIMEVPKAK | sp\|P05814\|CASB_HUMAN | beta-casein |
| 221 | QPAVVLPVPQPEIMEVPKAKDTVYT | sp\|P05814\|CASB_HUMAN | beta-casein |
| 222 | QPAVVLPVPQPEIMEVPKAKDTVYTK | sp\|P05814\|CASB_HUMAN | beta-casein |
| 223 | QPLAPVH | sp\|P05814\|CASB_HUMAN | beta-casein |
| 224 | QPLAPVHNPISV | sp\|P05814\|CASB_HUMAN | beta-casein |
| 225 | QQVPQPIP | sp\|P05814\|CASB_HUMAN | beta-casein |
| 226 | QVPQPIPQ | sp\|P05814\|CASB_HUMAN | beta-casein |
| 227 | QVPQPIPQTL | sp\|P05814\|CASB_HUMAN | beta-casein |
| 228 | RETIESL | sp\|P05814\|CASB_HUMAN | beta-casein |
| 229 | RETIESLSS | sp\|P05814\|CASB_HUMAN | beta-casein |
| 230 | RETIESLSSSEE | sp\|P05814\|CASB_HUMAN | beta-casein |
| 231 | RETIESLSSSEESI | sp\|P05814\|CASB_HUMAN | beta-casein |
| 232 | RETIESLSSSEESITE | sp\|P05814\|CASB_HUMAN | beta-casein |
| 233 | RETIESLSSSEESITEY | sp\|P05814\|CASB_HUMAN | beta-casein |
| 234 | RETIESLSSSEESITEYK | sp\|P05814\|CASB_HUMAN | beta-casein |
| 235 | RETIESLSSSEESITEYKQ | sp\|P05814\|CASB_HUMAN | beta-casein |
| 236 | RETIESLSSSEESITEYKQK | sp\|P05814\|CASB_HUMAN | beta-casein |
| 237 | RETIESLSSSEESITEYKQKVE | sp\|P05814\|CASB_HUMAN | beta-casein |
| 238 | RETIESLSSSEESITEYKQKVEK | sp\|P05814\|CASB_HUMAN | beta-casein |
| 239 | RETIESLSSSEESITEYKQKVEKV | sp\|P05814\|CASB_HUMAN | beta-casein |
| 240 | RETIESLSSSEESITEYKQKVEKVK | sp\|P05814\|CASB_HUMAN | beta-casein |
| 241 | RETIESLSSSEESITEYKQKVEKVKHE | sp\|P05814\|CASB_HUMAN | beta-casein |
| 242 | RETIESLSSSEESITEYKQKVEKVKHEDQQQG | sp\|P05814\|CASB_HUMAN | beta-casein |
| 243 | SEESITE | sp\|P05814\|CASB_HUMAN | beta-casein |
| 244 | SEESITEYK | sp\|P05814\|CASB_HUMAN | beta-casein |
| 245 | SEESITEYKQKVE | sp\|P05814\|CASB_HUMAN | beta-casein |
| 246 | SLSSSEESITE | sp\|P05814\|CASB_HUMAN | beta-casein |
| 247 | SLSSSEESITEYK | sp\|P05814\|CASB_HUMAN | beta-casein |
| 248 | SLSSSEESITEYKQKVEK | sp\|P05814\|CASB_HUMAN | beta-casein |
| 249 | SPTIPFF | sp\|P05814\|CASB_HUMAN | beta-casein |
| 250 | SPTIPFFD | sp\|P05814\|CASB_HUMAN | beta-casein |
| 251 | SPTIPFFDPQIPK | sp\|P05814\|CASB_HUMAN | beta-casein |
| 252 | SPTIPFFDPQIPKL | sp\|P05814\|CASB_HUMAN | beta-casein |
| 253 | SPTIPFFDPQIPKLTD | sp\|P05814\|CASB_HUMAN | beta-casein |
| 254 | SSEESITE | sp\|P05814\|CASB_HUMAN | beta-casein |
| 255 | SSEESITEY | sp\|P05814\|CASB_HUMAN | beta-casein |
| 256 | SSEESITEYK | sp\|P05814\|CASB_HUMAN | beta-casein |
| 257 | SSSEESITE | sp\|P05814\|CASB_HUMAN | beta-casein |
| 258 | SSSEESITEYK | sp\|P05814\|CASB_HUMAN | beta-casein |
| 259 | SSSEESITEYKQKVE | sp\|P05814\|CASB_HUMAN | beta-casein |
| 260 | SSSEESITEYKQKVEK | sp\|P05814\|CASB_HUMAN | beta-casein |
| 261 | SVPQPKVLPIPQQVVPYPQR | sp\|P05814\|CASB_HUMAN | beta-casein |
| 262 | SVPQPKVLPIPQQVVPYPQRAVPVQ | sp\|P05814\|CASB_HUMAN | beta-casein |
| 263 | SVPQPKVLPIPQQVVPYPQRAVPVQA | sp\|P05814\|CASB_HUMAN | beta-casein |
| 264 | TDLENLH | sp\|P05814\|CASB_HUMAN | beta-casein |
| 265 | TDLENLHLP | sp\|P05814\|CASB_HUMAN | beta-casein |
| 266 | TDLENLHLPLP | sp\|P05814\|CASB_HUMAN | beta-casein |
| 267 | TEYKQKVE | sp\|P05814\|CASB_HUMAN | beta-casein |
| 268 | TEYKQKVEKVKHED | sp\|P05814\|CASB_HUMAN | beta-casein |
| 269 | THQIYPVTQPLAPVHNPISV | sp\|P05814\|CASB_HUMAN | beta-casein |
| 270 | TIESLSSSEESITE | sp\|P05814\|CASB_HUMAN | beta-casein |
| 271 | TIESLSSSEESITEY | sp\|P05814\|CASB_HUMAN | beta-casein |
| 272 | TIESLSSSEESITEYK | sp\|P05814\|CASB_HUMAN | beta-casein |
| 273 | TIESLSSSEESITEYKQKVEK | sp\|P05814\|CASB_HUMAN | beta-casein |
| 274 | TQPLAPVH | sp\|P05814\|CASB_HUMAN | beta-casein |
| 275 | TQPLAPVHNPISV | sp\|P05814\|CASB_HUMAN | beta-casein |
| 276 | VEKVKHEDQQQGEDEHQDK | sp\|P05814\|CASB_HUMAN | beta-casein |
| 277 | VEKVKHEDQQQGEDEHQDKIYPS | sp\|P05814\|CASB_HUMAN | beta-casein |
| 278 | VEPIPYGFLPQ | sp\|P05814\|CASB_HUMAN | beta-casein |
| 279 | VKHEDQQQGEDEHQ | sp\|P05814\|CASB_HUMAN | beta-casein |
| 280 | VKHEDQQQGEDEHQD | sp\|P05814\|CASB_HUMAN | beta-casein |
| 281 | VKHEDQQQGEDEHQDK | sp\|P05814\|CASB_HUMAN | beta-casein |
| 282 | VKHEDQQQGEDEHQDKIYP | sp\|P05814\|CASB_HUMAN | beta-casein |
| 283 | VKHEDQQQGEDEHQDKIYPS | sp\|P05814\|CASB_HUMAN | beta-casein |
| 284 | VLPIPQ | sp\|P05814\|CASB_HUMAN | beta-casein |
| 285 | VLPIPQQV | sp\|P05814\|CASB_HUMAN | beta-casein |
| 286 | VLPIPQQVVP | sp\|P05814\|CASB_HUMAN | beta-casein |
| 287 | VLPIPQQVVPYP | sp\|P05814\|CASB_HUMAN | beta-casein |
| 288 | VLPIPQQVVPYPQ | sp\|P05814\|CASB_HUMAN | beta-casein |
| 289 | VLPIPQQVVPYPQR | sp\|P05814\|CASB_HUMAN | beta-casein |
| 290 | VLPIPQQVVPYPQRA | sp\|P05814\|CASB_HUMAN | beta-casein |
| 291 | VLPIPQQVVPYPQRAVPVQ | sp\|P05814\|CASB_HUMAN | beta-casein |
| 292 | VLPIPQQVVPYPQRAVPVQA | sp\|P05814\|CASB_HUMAN | beta-casein |
| 293 | VLPIPQQVVPYPQRAVPVQAL | sp\|P05814\|CASB_HUMAN | beta-casein |
| 294 | VLPVPQPEI | sp\|P05814\|CASB_HUMAN | beta-casein |
| 295 | VLPVPQPEIM | sp\|P05814\|CASB_HUMAN | beta-casein |
| 296 | VLPVPQPEIME | sp\|P05814\|CASB_HUMAN | beta-casein |
| 297 | VLPVPQPEIMEVPK | sp\|P05814\|CASB_HUMAN | beta-casein |
| 298 | VMPVLKSPTIP | sp\|P05814\|CASB_HUMAN | beta-casein |
| 299 | VPKAKDTVYT | sp\|P05814\|CASB_HUMAN | beta-casein |
| 300 | VPKAKDTVYTKG | sp\|P05814\|CASB_HUMAN | beta-casein |
| 301 | VPQPIP | sp\|P05814\|CASB_HUMAN | beta-casein |
| 302 | VPQPIPQ | sp\|P05814\|CASB_HUMAN | beta-casein |
| 303 | VPQPKVLPIPQQV | sp\|P05814\|CASB_HUMAN | beta-casein |
| 304 | VPYPQRAVPVQA | sp\|P05814\|CASB_HUMAN | beta-casein |
| 305 | VTQPLAPVHNPISV | sp\|P05814\|CASB_HUMAN | beta-casein |
| 306 | VVLPVPQPEIME | sp\|P05814\|CASB_HUMAN | beta-casein |
| 307 | VVLPVPQPEIMEVPK | sp\|P05814\|CASB_HUMAN | beta-casein |
| 308 | VVLPVPQPEIMEVPKA | sp\|P05814\|CASB_HUMAN | beta-casein |
| 309 | VVLPVPQPEIMEVPKAK | sp\|P05814\|CASB_HUMAN | beta-casein |
| 310 | VVLPVPQPEIMEVPKAKDT | sp\|P05814\|CASB_HUMAN | beta-casein |
| 311 | VVLPVPQPEIMEVPKAKDTVYT | sp\|P05814\|CASB_HUMAN | beta-casein |
| 312 | VVLPVPQPEIMEVPKAKDTVYTK | sp\|P05814\|CASB_HUMAN | beta-casein |
| 313 | VVLPVPQPEIMEVPKAKDTVYTKG | sp\|P05814\|CASB_HUMAN | beta-casein |
| 314 | VVLPVPQPEIMEVPKAKDTVYTKGR | sp\|P05814\|CASB_HUMAN | beta-casein |
| 315 | VVPYPQRAVPVQ | sp\|P05814\|CASB_HUMAN | beta-casein |
| 316 | VVPYPQRAVPVQA | sp\|P05814\|CASB_HUMAN | beta-casein |
| 317 | YPVTQPLAPVH | sp\|P05814\|CASB_HUMAN | beta-casein |
| 318 | YPVTQPLAPVHNPISV | sp\|P05814\|CASB_HUMAN | beta-casein |
| 507 | DQQQGEDEHQDKIYP | sp\|P05814\|CASB_HUMAN | beta-casein |
| 508 | EESITEYKQKV | sp\|P05814\|CASB_HUMAN | beta-casein |
| 509 | EVPKAKDTVYTKG | sp\|P05814\|CASB_HUMAN | beta-casein |
| 510 | AQPAVVLPVPQPEIMEVPKAK | sp\|P05814\|CASB_HUMAN | beta-casein |
| 511 | LPVPQPEIMEVPKA | sp\|P05814\|CASB_HUMAN | beta-casein |
| 319 | QLAPIWDKLGETYKDH | sp\|P07237\|PDIA1_HUMAN | protein disulfide- |
| | | | isomerase |
| 320 | ANPAVVRPHAQIPQRQY | sp\|P07498\|CASK_HUMAN | kappa-casein |
| 321 | HPPTVVR | sp\|P07498\|CASK_HUMAN | kappa-casein |
| 322 | LPNSHPPT | sp\|P07498\|CASK_HUMAN | kappa-casein |
| 323 | LPNSHPPTV | sp\|P07498\|CASK_HUMAN | kappa-casein |
| 324 | LPNSHPPTVVR | sp\|P07498\|CASK_HUMAN | kappa-casein |
| 325 | TTTVAVTPP | sp\|P07498\|CASK_HUMAN | kappa-casein |
| 326 | TYYANPAVVRPHA | sp\|P07498\|CASK_HUMAN | kappa-casein |
| 327 | TYYANPAVVRPHAQIP | sp\|P07498\|CASK_HUMAN | kappa-casein |
| 328 | TYYANPAVVRPHAQIPQR | sp\|P07498\|CASK_HUMAN | kappa-casein |
| 329 | TYYANPAVVRPHAQIPQRQY | sp\|P07498\|CASK_HUMAN | kappa-casein |
| 330 | YANPAVVRPHAQIPQR | sp\|P07498\|CASK_HUMAN | kappa-casein |
| 331 | IYDKTYAGGRL | sp\|P07996\|TSP1_HUMAN | thrombospondin-1 |
| 332 | DDEEKPKI | sp\|P08238\|HS90B_HUMAN | heat shock protein |
| | | | HSP 90-beta |
| 333 | AGGGGL | sp\|P0C0L4\|CO4A_HUMAN | complement C4-A |
| 334 | DDPDAPLQPVTPLQLFEGRRN | sp\|P0C0L4\|CO4A_HUMAN | complement C4-A |
| 335 | ELTSWYFVS | sp\|P0C0L4\|CO4A_HUMAN | complement C4-A |
| 336 | KINVKVGGNSKGTLKVLRTYNVLDMKNTTC | sp\|P0C0L4\|CO4A_HUMAN | complement C4-A |
| 337 | AIPVAQDLNAPS | sp\|P10451\|OSTP_HUMAN | osteopontin |
| 338 | AIPVAQDLNAPSD | sp\|P10451\|OSTP_HUMAN | osteopontin |
| 339 | ATDEDITSH | sp\|P10451\|OSTP_HUMAN | osteopontin |
| 340 | DIQYPDATDEDITSH | sp\|P10451\|OSTP_HUMAN | osteopontin |
| 341 | DQSAETHSHKQSRLY | sp\|P10451\|OSTP_HUMAN | osteopontin |
| 342 | EDITSHME | sp\|P10451\|OSTP_HUMAN | osteopontin |
| 343 | ESEELNGAYK | sp\|P10451\|OSTP_HUMAN | osteopontin |
| 344 | GDSVVYGLR | sp\|P10451\|OSTP_HUMAN | osteopontin |
| 345 | HELDSASSEVN | sp\|P10451\|OSTP_HUMAN | osteopontin |
| 346 | IPVAQD | sp\|P10451\|OSTP_HUMAN | osteopontin |
| 347 | IPVAQDLNAPS | sp\|P10451\|OSTP_HUMAN | osteopontin |
| 348 | IPVKQADS | sp\|P10451\|OSTP_HUMAN | osteopontin |
| 349 | IPVKQADSG | sp\|P10451\|OSTP_HUMAN | osteopontin |
| 350 | ISHELDSASSEVN | sp\|P10451\|OSTP_HUMAN | osteopontin |
| 351 | NKYPDAVAT | sp\|P10451\|OSTP_HUMAN | osteopontin |
| 352 | RISHELDSASSEVN | sp\|P10451\|OSTP_HUMAN | osteopontin |
| 353 | RPDIQYPDAT | sp\|P10451\|OSTP_HUMAN | osteopontin |
| 354 | RPDIQYPDATD | sp\|P10451\|OSTP_HUMAN | osteopontin |
| 355 | RPDIQYPDATDEDIT | sp\|P10451\|OSTP_HUMAN | osteopontin |
| 356 | RPDIQYPDATDEDITSH | sp\|P10451\|OSTP_HUMAN | osteopontin |
| 357 | RPDIQYPDATDEDITSHMESEELNGAYK | sp\|P10451\|OSTP_HUMAN | osteopontin |
| 358 | RRPDIQYPDATDEDIT | sp\|P10451\|OSTP_HUMAN | osteopontin |
| 359 | RRPDIQYPDATDEDITSH | sp\|P10451\|OSTP_HUMAN | osteopontin |
| 360 | RRPDIQYPDATDEDITSHMESEELNGAYK | sp\|P10451\|OSTP_HUMAN | osteopontin |
| 361 | SEELNGAYK | sp\|P10451\|OSTP_HUMAN | osteopontin |
| 362 | SHELDSASSEVN | sp\|P10451\|OSTP_HUMAN | osteopontin |
| 363 | SKSKKFRRPDIQYPDATD | sp\|P10451\|OSTP_HUMAN | osteopontin |
| 364 | SKSKKFRRPDIQYPDATDEDITSH | sp\|P10451\|OSTP_HUMAN | osteopontin |
| 365 | SKSKKFRRPDIQYPDATDEDITSHMESEELNGAYK | sp\|P10451\|OSTP_HUMAN | osteopontin |
| 366 | TYDGRGDSVVYGLR | sp\|P10451\|OSTP_HUMAN | osteopontin |
| 367 | YPDATDEDITSH | sp\|P10451\|OSTP_HUMAN | osteopontin |
| 516 | ATDEDITSHMESEELNGAYK | sp\|P10451\|OSTP_HUMAN | osteopontin |
| 517 | EDITSHMESEELNGAYK | sp\|P10451\|OSTP_HUMAN | osteopontin |
| 518 | DIQYPDATDEDITSHMESEELNGAYK | sp\|P10451\|OSTP_HUMAN | osteopontin |
| 519 | DDQSAETHSHKQSRLY | sp\|P10451\|OSTP_HUMAN | osteopontin |
| 368 | DRSPYEKVSAGNGGSSLS | sp\|P15941\|MUC1_HUMAN | mucin-1 |
| 369 | SPYEKVSAGNGGSS | sp\|P15941\|MUC1_HUMAN | mucin-1 |
| 370 | SPYEKVSAGNGGSSL | sp\|P15941\|MUC1_HUMAN | mucin-1 |
| 371 | SPYEKVSAGNGGSSLS | sp\|P15941\|MUC1_HUMAN | mucin-1 |
| 372 | STDRSPYEKVSAGNGGSSLSY | sp\|P15941\|MUC1_HUMAN | mucin-1 |
| 373 | TDRSPYEKVSAGNGGSSLS | sp\|P15941\|MUC1_HUMAN | mucin-1 |
| 374 | TDRSPYEKVSAGNGGSSLSY | sp\|P15941\|MUC1_HUMAN | mucin-1 |
| 375 | TDRSPYEKVSAGNGGSSLSYTNPAVAATSANL | sp\|P15941\|MUC1_HUMAN | mucin-1 |
| 376 | TNPAVAATSANL | sp\|P15941\|MUC1_HUMAN | mucin-1 |
| 377 | SGNHPITVHCSAGAGRTGTFCALSTV | sp\|P18433\|PTPRA_HUMAN | receptor-type |
| | | | tyrosine-protein |
| | | | phosphatase alpha |
| 378 | EGGFVEGVNK | sp\|P19835\|CEL_HUMAN | bile salt-activated |
| | | | lipase |
| 379 | KLGAVYTEGGFVEGVNK | sp\|P19835\|CEL_HUMAN | bile salt-activated |
| | | | lipase |
| 380 | RQKASLTNVTDPSLDLTSLSLEVGCGAPAPV | sp\|P22079\|PERL_HUMAN | lactoperoxidase |
| 381 | DPSKPSSNVAGVVII | sp\|P22897\|MRC1_HUMAN | macrophage mannose |
| | | | receptor 1 |
| 382 | DPSKPSSNVAGVVIIV | sp\|P22897\|MRC1_HUMAN | macrophage mannose |
| | | | receptor 1 |
| 383 | QRHNSSIN | sp\|P22897\|MRC1_HUMAN | macrophage mannose |
| | | | receptor 1 |
| 384 | SLWNKDPLTSVSYQINSKS | sp\|P22897\|MRC1_HUMAN | macrophage mannose |
| | | | receptor 1 |
| 385 | AEMKLR | sp\|P24821\|TENA_HUMAN | tenascin |
| 386 | YRLNYSLPT | sp\|P24821\|TENA_HUMAN | tenascin |
| 387 | EKQTDEIKDTR | sp\|P47710\|CASA1_HUMAN | alpha-S1-casein |
| 388 | LQNPSESSEPIPLE | sp\|P47710\|CASA1_HUMAN | alpha-S1-casein |
| 389 | LQNPSESSEPIPLESR | sp\|P47710\|CASA1_HUMAN | alpha-S1-casein |
| 390 | LQNPSESSEPIPLESREEYMNGMN | sp\|P47710\|CASA1_HUMAN | alpha-S1-casein |
| 391 | MNRQRNILR | sp\|P47710\|CASA1_HUMAN | alpha-S1-casein |
| 392 | NILREKQTDE | sp\|P47710\|CASA1_HUMAN | alpha-S1-casein |
| 393 | NILREKQTDEIKDTR | sp\|P47710\|CASA1_HUMAN | alpha-S1-casein |
| 394 | NPSESSEPIP | sp\|P47710\|CASA1_HUMAN | alpha-S1-casein |
| 395 | NPSESSEPIPLESR | sp\|P47710\|CASA1_HUMAN | alpha-S1-casein |
| 396 | NPSESSEPIPLESREEYMNGMN | sp\|P47710\|CASA1_HUMAN | alpha-S1-casein |
| 397 | NYEKNNVML | sp\|P47710\|CASA1_HUMAN | alpha-S1-casein |
| 398 | QRNILREKQTDEIKDTR | sp\|P47710\|CASA1_HUMAN | alpha-S1-casein |
| 399 | RLQNPSE | sp\|P47710\|CASA1_HUMAN | alpha-S1-casein |
| 400 | RLQNPSESSEPIP | sp\|P47710\|CASA1_HUMAN | alpha-S1-casein |
| 401 | RLQNPSESSEPIPLE | sp\|P47710\|CASA1_HUMAN | alpha-S1-casein |
| 402 | RLQNPSESSEPIPLESR | sp\|P47710\|CASA1_HUMAN | alpha-S1-casein |
| 403 | RLQNPSESSEPIPLESREEYMNGM | sp\|P47710\|CASA1_HUMAN | alpha-S1-casein |
| 404 | RLQNPSESSEPIPLESREEYMNGMN | sp\|P47710\|CASA1_HUMAN | alpha-S1-casein |
| 405 | RLQNPSESSEPIPLESREEYMNGMNR | sp\|P47710\|CASA1_HUMAN | alpha-S1-casein |
| 406 | RPKLPLR | sp\|P47710\|CASA1_HUMAN | alpha-S1-casein |
| 407 | RPKLPLRYPE | sp\|P47710\|CASA1_HUMAN | alpha-S1-casein |
| 408 | RPKLPLRYPERLQ | sp\|P47710\|CASA1_HUMAN | alpha-S1-casein |
| 409 | RPKLPLRYPERLQNPSESSEPIPLESREEYMNGMN | sp\|P47710\|CASA1_HUMAN | alpha-S1-casein |
| 410 | YEKNNVML | sp\|P47710\|CASA1_HUMAN | alpha-S1-casein |
| 411 | AIYASKAVGEPP | sp\|P47989\|XDH_HUMAN | xanthine |
| | | | dehydrogenase/oxidase |
| 412 | DTSEAKKV | sp\|P47989\|XDH_HUMAN | xanthine |
| | | | dehydrogenase/oxidase |
| 413 | VPANRIVV | sp\|P47989\|XDH_HUMAN | xanthine |
| | | | dehydrogenase/oxidase |
| 414 | TRELDELMASLSDFKIQGLEQ | sp\|P49023\|PAXI_HUMAN | paxillin |
| 415 | WPRDGGRSSPGGQDEGGFMAQGKTGSSSPPG | sp\|P49023\|PAXI_HUMAN | paxillin |
| 416 | FPAPRGTPLISP | sp\|P49327\|FAS_HUMAN | fatty acid synthase |
| 417 | SGVVGLVNC | sp\|P49327\|FAS_HUMAN | fatty acid synthase |
| 418 | AESSKPTAGGSRSQ | sp\|P49454\|CENPF_HUMAN | centromere protein F |
| 419 | LPPPPPPPP | sp\|P55196\|AFAD_HUMAN | afadin |
| 420 | GRGGRGGSRARNLPLPPPPP | sp\|P61978\|HNRPK_HUMAN | heterogeneous nuclear |
| | | | ribonucleoprotein K |
| 421 | LPLPPPPPP | sp\|P61978\|HNRPK_HUMAN | heterogeneous nuclear |
| | | | ribonucleoprotein K |
| 422 | TKIIEGGAAHKDGRLQ | sp\|P78352\|DLG4_HUMAN | disks large homolog 4 |
| 423 | ADINTPLTTSSGNLHGQPVSFLLREL | sp\|P78540\|ARGI2_HUMAN | arginase-2, |
| | | | mitochondrial |
| 424 | HHAAIEILQNAPEDVTLVISQPKEK | sp\|Q12923\|PTN13_HUMAN | tyrosine-protein |
| | | | phosphatase non- |
| | | | receptor type 13 |
| 425 | VPLPPARPPTRD | sp\|Q13191\|CBLB_HUMAN | E3 ubiquitin-protein |
| | | | ligase CBL-B |
| 426 | ADTLHSKLIPTQPSQGAP | sp\|Q13410\|BT1A1_HUMAN | butyrophilin |
| | | | subfamily 1 member A1 |
| 427 | APFDVIGPPEPILA | sp\|Q13410\|BT1A1_HUMAN | butyrophilin |
| | | | subfamily 1 member A1 |
| 428 | APRDADTLHSKLIPTQPSQGAP | sp\|Q13410\|BT1A1_HUMAN | butyrophilin |
| | | | subfamily 1 member A1 |
| 429 | DGPERVTVIANAQDLS | sp\|Q13410\|BT1A1_HUMAN | butyrophilin |
| | | | subfamily 1 member A1 |
| 430 | DGREQEAEQMPEY | sp\|Q13410\|BT1A1_HUMAN | butyrophilin |
| | | | subfamily 1 member A1 |
| 431 | DGREQEAEQMPEYR | sp\|Q13410\|BT1A1_HUMAN | butyrophilin |
| | | | subfamily 1 member A1 |
| 432 | DGREQEAEQMPEYRG | sp\|Q13410\|BT1A1_HUMAN | butyrophilin |
| | | | subfamily 1 member A1 |
| 433 | DGREQEAEQMPEYRGR | sp\|Q13410\|BT1A1_HUMAN | butyrophilin |
| | | | subfamily 1 member A1 |
| 434 | DVIGPP | sp\|Q13410\|BT1A1_HUMAN | butyrophilin |
| | | | subfamily 1 member A1 |
| 435 | EDSAPRDADTLH | sp\|Q13410\|BT1A1_HUMAN | butyrophilin |
| | | | subfamily 1 member A1 |
| 436 | EIPLSPMGEDSAPR | sp\|Q13410\|BT1A1_HUMAN | butyrophilin |
| | | | subfamily 1 member A1 |
| 437 | EIPLSPMGEDSAPRDADTLH | sp\|Q13410\|BT1A1_HUMAN | butyrophilin |
| | | | subfamily 1 member A1 |
| 438 | GRATLVQDGIAK | sp\|Q13410\|BT1A1_HUMAN | butyrophilin |
| | | | subfamily 1 member A1 |
| 439 | GRATLVQDGIAKGRVA | sp\|Q13410\|BT1A1_HUMAN | butyrophilin |
| | | | subfamily 1 member A1 |
| 440 | GREQEAEQMPEYR | sp\|Q13410\|BT1A1_HUMAN | butyrophilin |
| | | | subfamily 1 member A1 |
| 441 | GREQEAEQMPEYRGR | sp\|Q13410\|BT1A1_HUMAN | butyrophilin |
| | | | subfamily 1 member A1 |
| 442 | IPLSPMGEDS | sp\|Q13410\|BT1A1_HUMAN | butyrophilin |
| | | | subfamily 1 member A1 |
| 443 | IPLSPMGEDSAPR | sp\|Q13410\|BT1A1_HUMAN | butyrophilin |
| | | | subfamily 1 member A1 |
| 444 | IPLSPMGEDSAPRDADTLH | sp\|Q13410\|BT1A1_HUMAN | butyrophilin |
| | | | subfamily 1 member A1 |
| 445 | KEIPLSPMGED | sp\|Q13410\|BT1A1_HUMAN | butyrophilin |
| | | | subfamily 1 member A1 |
| 446 | KEIPLSPMGEDSAPR | sp\|Q13410\|BT1A1_HUMAN | butyrophilin |
| | | | subfamily 1 member A1 |
| 447 | KEIPLSPMGEDSAPRDADT | sp\|Q13410\|BT1A1_HUMAN | butyrophilin |
| | | | subfamily 1 member A1 |
| 448 | KEIPLSPMGEDSAPRDADTLH | sp\|Q13410\|BT1A1_HUMAN | butyrophilin |
| | | | subfamily 1 member A1 |
| 449 | KEIPLSPMGEDSAPRDADTLHS | sp\|Q13410\|BT1A1_HUMAN | butyrophilin |
| | | | subfamily 1 member A1 |
| 450 | KEIPLSPMGEDSAPRDADTLHSK | sp\|Q13410\|BT1A1_HUMAN | butyrophilin |
| | | | subfamily 1 member A1 |
| 451 | KEIPLSPMGEDSAPRDADTLHSKLIPTQPSQ | sp\|Q13410\|BT1A1_HUMAN | butyrophilin |
| | | | subfamily 1 member A1 |
| 452 | KEIPLSPMGEDSAPRDADTLHSKLIPTQPSQGAP | sp\|Q13410\|BT1A1_HUMAN | butyrophilin |
| | | | subfamily 1 member A1 |
| 453 | LPLAGP | sp\|Q13410\|BT1A1_HUMAN | butyrophilin |
| | | | subfamily 1 member A1 |
| 454 | QDLSKEIPLSPMGEDSAPRDADTLH | sp\|Q13410\|BT1A1_HUMAN | butyrophilin |
| | | | subfamily 1 member A1 |
| 455 | SKLIPTQPSQG | sp\|Q13410\|BT1A1_HUMAN | butyrophilin |
| | | | subfamily 1 member A1 |
| 456 | SKLIPTQPSQGAP | sp\|Q13410\|BT1A1_HUMAN | butyrophilin |
| | | | subfamily 1 member A1 |
| 457 | SPMGEDSAPRDADTLH | sp\|Q13410\|BT1A1_HUMAN | butyrophilin |
| | | | subfamily 1 member A1 |
| 458 | TLVQDGIAK | sp\|Q13410\|BT1A1_HUMAN | butyrophilin |
| | | | subfamily 1 member A1 |
| 459 | TLVQDGIAKGRVA | sp\|Q13410\|BT1A1_HUMAN | butyrophilin |
| | | | subfamily 1 member A1 |
| 460 | SPLPHSSPPTAAVATTSITTA | sp\|Q14160\|SCRIB_HUMAN | protein scribble |
| | | | homolog |
| 461 | LPSKTPPPPPPKTTR | sp\|Q14185\|DOCK1_HUMAN | dedicator of |
| | | | cytokinesis protein 1 |
| 462 | AAFKTLSGAQDSEAAFAKLDQKDLVLPTQALPASP | sp\|Q15554\|TERF2_HUMAN | telomeric repeat- |
| | | | binding factor 2 |
| 463 | PPPPPPPPLLPGSSAEPPPPPP | sp\|Q27J81\|INF2_HUMAN | inverted formin-2 |
| 464 | SEGDGGRL | sp\|Q53EL6\|PDCD4_HUMAN | programmed cell death |
| | | | protein 4 |
| 465 | LEKQLESSQARKAMEEFFSD | sp\|Q5T4S7\|UBR4_HUMAN | E3 ubiquitin-protein |
| | | | ligase UBR4 |
| 466 | EKLSALKISN | sp\|Q659A1\|NARG2_HUMAN | NMDA receptor- |
| | | | regulated protein 2 |
| 467 | KVNMISREQFDTLTPEPP | sp\|Q6PKG0\|LARP1_HUMAN | 1a-related protein 1 |
| 468 | AAEPPTLISPQAPASSPSSLSTSPPEV | sp\|Q6UWI2\|PARM1_HUMAN | prostate androgen- |
| | | | regulated mucin-like |
| | | | protein 1 |
| 469 | RPPPPPPP | sp\|Q70EK9\|UBP51_HUMAN | ubiquitin carboxyl- |
| | | | terminal hydrolase 51 |
| 470 | TKVGEIFSAAGAAF | sp\|Q8IXM2\|BAP18_HUMAN | chromatin complexes |
| | | | subunit BAP18 |
| 471 | LNLSENPAMTEGLLRAQVDSSFLSLYDSHVAKEILLRVLTLFQNIKNCLKI | sp\|Q8N2F6\|ARM10_HUMAN | Armadillo repeat- |
| | | | containing protein 10 |
| 472 | QRTSSIATALNTSGAGGSRP | sp\|Q8N4C8\|MINK1_HUMAN | misshapen-like kinase |
| | | | 1 |
| 473 | SLDDIDLSALRDP | sp\|Q8N4C8\|MINK1_HUMAN | misshapen-like kinase |
| | | | 1 |
| 474 | SVALPPPPGPPPPP | sp\|Q8N8S7\|ENAH_HUMAN | protein enabled |
| | | | homolog |
| 475 | PPAPPPPPP | sp\|Q8NFC6\|BD1L1_HUMAN | biorientation of |
| | | | chromosomes in cell |
| | | | division protein 1- |
| | | | like 1 |
| 476 | PPPAPPPPP | sp\|Q8NFC6\|BD1L1_HUMAN | biorientation of |
| | | | chromosomes in cell |
| | | | division protein 1- |
| | | | like 1 |
| 477 | DLLVEILMRPTIS | sp\|Q8TEL6\|TP4AP_HUMAN | short transient |
| | | | receptor potential |
| | | | channel 4-associated |
| | | | protein |
| 478 | ERPPPP | sp\|Q92625\|ANS1A_HUMAN | ankyrin repeat and |
| | | | SAM domain-containing |
| | | | protein 1A |
| 479 | NRNDQEATLEMLFPSRTT | sp\|Q92918\|M4K1_HUMAN | mitogen-activated |
| | | | protein kinase kinase |
| | | | kinase kinase 1 |
| 480 | IIGGFWLG | sp\|Q96A29\|FUCT1_HUMAN | GDP-fucose |
| | | | transporter 1 |
| 481 | PMRRKSGPSCKHCKDDVNRLCRVCACHLCGGRQD | sp\|Q96T88\|UHRF1_HUMAN | E3 ubiquitin-protein |
| | | | ligase UHRF1 |
| 482 | TTLIQYTSN | sp\|Q99102\|MUC4_HUMAN | mucin-4 |
| 483 | AEMDKSSQETQRSEHKTH | sp\|Q99541\|PLIN2_HUMAN | perilipin-2 |
| 484 | DQGAEMDKSSQETQRSEHKTH | sp\|Q99541\|PLIN2_HUMAN | perilipin-2 |
| 485 | EMDKSSQETQRSEHKTH | sp\|Q99541\|PLIN2_HUMAN | perilipin-2 |
| 486 | LPIIQKLEPQ | sp\|Q99541\|PLIN2_HUMAN | perilipin-2 |
| 487 | LPIIQKLEPQIA | sp\|Q99541\|PLIN2_HUMAN | perilipin-2 |
| 488 | LVSSGVENALT | sp\|Q99541\|PLIN2_HUMAN | perilipin-2 |
| 489 | VMDKTKGAV | sp\|Q99541\|PLIN2_HUMAN | perilipin-2 |
| 490 | KGYPRNISHNWMHCRPR | sp\|Q99542\|MMP19_HUMAN | matrix |
| | | | metalloproteinase-19 |
| 491 | WGRGNFTEGKVPH | sp\|Q9BYT3\|STK33_HUMAN | serine/threonine- |
| | | | protein kinase 33 |
| 492 | CAQRDNPRASSPSRATRDN | sp\|Q9H2D6\|TARA_HUMAN | TRIO and F-actin- |
| | | | binding protein |
| 493 | PRPLHPPPPPP | sp\|Q9UKV3\|ACINU_HUMAN | apoptotic chromatin |
| | | | condensation inducer |
| | | | in the nucleus |
| 494 | DSSVASQIT | sp\|Q9UKZ1\|CB029_HUMAN | UPF0760 protein |
| | | | C2orf29 |
| 495 | LAKGNAGKVNLPKELPADAVNLTIPASLDLSPLL | sp\|Q9UM63\|PLAL1_HUMAN | zinc finger protein |
| | | | PLAGL1 |
| 496 | LGEKLGGNVVVSL | sp\|Q9Y281\|COF2_HUMAN | cofilin-2 |
| 497 | TVLGNGSSLSLPEGQSLRLVCAV | sp\|Q9Y336\|SIGL9_HUMAN | sialic acid-binding |
| | | | Ig-like lectin 9 |
| 498 | TVLGNGSSLSLPEGQSLRLVCAVDAVD | sp\|Q9Y336\|SIGL9_HUMAN | sialic acid-binding |
| | | | Ig-like lectin 9 |
| 499 | PSAPTAHPQPRPPQGPLALPGPSYAGNSP | sp\|Q9Y4B6\|VPRBP_HUMAN | protein VPRBP |
| 500 | MIYTYSGLFCVTVN | sp\|Q9Y623\|MYH4_HUMAN | myosin-4 |
| 501 | DHLVCFLPGTLALGVY | tr\|B3KQC5\|B3KQC5_HUMAN | Endoplasmic reticulum |
| | | | mannosyl- |
| | | | oligosaccharide 1,2- |
| | | | alpha-mannosidase |
| | | | (MAN1B1) |
| 502 | QLVSLLHMSL | tr\|B4DWJ0\|B4DWJ0_HUMAN | cDNA FLJ57167, highly |
| | | | similar to Etoposide- |
| | | | induced protein 2.4 |
| 514 | PDPAKQTDRV | sp\|Q15262\|PTPRK_HUMAN | Receptor-type |
| | | | tyrosine-protein |
| | | | phosphatase kappa |
| 515 | VTAEKAPPPPPP | sp\|O60346\|PHLP1_HUMAN | PH domain leucine- |
| | | | rich repeat- |
| | | | containing protein |
| | | | phosphatase 1 |
| 520 | AKSQTEQTQPLSLSLKPDPLAHLSM | sp\|Q9NQB0\|TF7L2_HUMAN | Transcription factor |
| | | | 7-like 2 |
| 521 | SFRVRASSDGEGTMSRP | sp\|P35568\|IRS1_HUMAN | Insulin receptor |
| | | | substrate 1 |
| 522 | CSSPNDSEHGP | sp\|Q8WUI4\|HDAC7_HUMAN | Histone deacetylase 7 |
| 523 | QWLHTQVGVH | sp\|Q96JM4\|LRIQ1_HUMAN | Leucine-rich repeat |
| | | | and IQ domain- |
| | | | containing protein 1 |
| 524 | LAGDALLSLLAGDLGVEVPSAVPRPTLEPAEQL | sp\|Q6P531\|GGT6_HUMAN | Gamma- |
| | | | glutamyltransferase 6 |
| 525 | EHSESTLNVM | sp\|P42356\|PI4KA_HUMAN | Phosphatidylinositol |
| | | | 4-kinase alpha |
| 526 | GLNYHKRCAFSIPNNCSGARKRRLSSTSLA | tr\|Q8NCK8\|Q8NCK8_HUMAN | cDNA FLJ38565 fis, |
| | | | clone HCHON2005048, |
| | | | highly similar to |
| | | | Serine/threonine- |
| | | | protein kinase D2 (EC |
| | | | 2.7.11.13) |
| 527 | AVSEHQLLHDKGKSIQDLR | sp\|P12272\|PTHR_HUMAN | Parathyroid hormone- |
| | | | related protein |
| 528 | IIIGIGNSGGDLAVEISQTA | tr\|Q9HA79\|Q9HA79_HUMAN | Flavin containing |
| | | | monooxygenase 5, |
| | | | isoform CRA_c |
| 529 | THTVTY | sp\|O75369\|FLNB_HUMAN | Filamin-B |
| 530 | GPEAAKSDETAAK | sp\|P04792\|HSPB1_HUMAN | Heat shock protein |
| | | | beta-1 |
| 531 | GGGGGGGGGGGGGGGGEAGAVAPYGYTR | tr\|Q9UN21\|Q9UN21_HUMAN | Androgen receptor |
| 532 | SPPPPPPPP | sp\|Q8IZP0\|ABI1_HUMAN | Abl interactor 1 |
| 533 | PPPLPPPPPP | sp\|Q96JH7\|VCIP1_HUMAN | Deubiquitinating |
| | | | protein VCIP135 |
| 534 | IPPPPPP | sp\|O60610\|DIAP1_HUMAN | Protein diaphanous |
| | | | homolog 1 |
| 535 | YPPPPPPPPP | sp\|Q92841\|DDX17_HUMAN | Probable ATP- |
| | | | dependent RNA |
| | | | helicase DDX17 |

3. Formulation and Administration

The antibacterial peptides can be prepared as a variety of pharmaceutical formulations for administration to a patient, including liquid and solid form preparations.

Compositions comprising one or more of the antibacterial peptides, e.g., 1, 2, 3, 4, 5, 6, 7, 8, 9, 10, 20, 30, 40, 50, 60, 70, 80, 90, 100, 150, 200, 250, 300, 350, 400, 450, 500, 503 peptides described herein, are useful for parenteral, topical, oral, or local administration, including by aerosol or transdermally, for prophylactic and/or therapeutic treatment. The pharmaceutical compositions can be administered in a variety of unit dosage forms depending upon the method of administration. For example, unit dosage forms suitable for oral administration include powder, tablets, pills, capsules and lozenges. It is recognized that the polypeptides and pharmaceutical compositions of this invention, when administered orally, must be protected from digestion. This is typically accomplished either by complexing the polypeptide with a composition to render it resistant to acidic and enzymatic hydrolysis or by packaging the protein in an appropriately resistant carrier such as a liposome. Means of protecting proteins from digestion are well known in the art.

Compositions comprising the antibacterial peptides are particularly useful for parenteral administration, such as intravenous administration or administration into a body cavity or lumen of an organ. The compositions for administration will commonly comprise a solution of the polypeptide comprising the polypeptide dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of polypeptide in these formulations can vary widely, and will be selected primarily based on fluid volumes, viscosities, body weight and the like in accordance with the particular mode of administration selected and the patient's needs.

Liquid form pharmaceutical preparations can include solutions, suspensions, and emulsions, for example, water or water/propylene glycol solutions. Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents. For parenteral injection, liquid preparations can be formulated in solution in aqueous polyethylene glycol solution. Transdermal administration can be performed using suitable carriers. If desired, apparatuses designed to facilitate transdermal delivery can be employed. Suitable carriers and apparatuses are well known in the art, as exemplified by U.S. Pat. Nos. 6,635,274, 6,623,457, 6,562,004, and 6,274,166.

In some embodiments, the antibacterial peptides are formulated as a nanoparticle. Peptide nanoparticles and methods for their preparation are known in the art and described, e.g., in U.S. Patent Publication No. 2006/0251726, U.S. Patent Publication No. 2004/0126900, U.S. Patent Publication No. 2005/0112089, U.S. Patent Publication No. 2010/0172943, U.S. Patent Publication No. 2010/0055189, U.S. Patent Publication No. 2009/0306335, U.S. Patent Publication No. 2009/0156480, and U.S. Patent Publication No. 2008/0213377, each of which is hereby incorporated herein by reference in its entirety for all purposes. Further nanoparticle formulations that find use are described, e.g., in Emerich and Thanos, Curr Opin Mol Ther (2008) 10(2):132-9; Kogan, et al., Nanomedicine (2007) 2(3):287-306; Zhang, et al., Bioconjug Chem (2008) 19(1):145-152; Scarberry, et al., J Am Chem Soc (2008) 130(31):10258-10262; and Fraysse-Ailhas, et al., Eur Cells Materials (2007) 14(Suppl. 3):115. As appropriate, amino acid sequences may be added to either or both the N-terminus and the C-terminus of the peptide ligands in order to allow assembly and formation of the peptide nanoparticle.

Also contemplated are solid form pharmaceutical formulations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

In varying embodiments, the peptide or mixture of peptides are formulated for topical administration. A variety of solid, semisolid and liquid vehicles have been known in the art for years for topical application of agents to the skin. Such vehicles include creams, lotions, gels, balms, oils, ointments and sprays. See, e.g., Provost C. “Transparent oil-water gels: a review,” Int J Cosmet Sci. 8:233-247 (1986), Katz and Poulsen, Concepts in biochemical pharmacology, part I. In: Brodie B B, Gilette J R, eds. Handbook of Experimental Pharmacology. Vol. 28. New York, N.Y.: Springer; 107-174 (1971), and Hadgcraft, “Recent progress in the formulation of vehicles for topical applications,” Br J. Dermatol., 81:386-389 (1972). It is presumed that the person of skill is familiar with these various vehicles and preparations and they need not be described in detail herein.

The antibacterial peptide or mixture of peptides can be mixed into such modalities (creams, lotions, gels, etc.) for topical administration. In general, the concentration of the agents provides a gradient which drives the agent into the skin. Standard ways of determining flux of drugs into the skin, as well as for modifying agents to speed or slow their delivery into the skin are well known in the art and taught, for example, in Osborne and Amann, eds., Topical Drug Delivery Formulations, Marcel Dekker, 1989. The use of dermal drug delivery agents in particular is taught in, for example, Ghosh et al., eds., Transdermal and Topical Drug Delivery Systems, CRC Press, (Boca Raton, Fla., 1997).

In some embodiments, the agents are in a cream. Typically, the cream comprises one or more hydrophobic lipids, with other agents to improve the “feel” of the cream or to provide other useful characteristics. In one embodiment, for example, a cream of the invention may contain 0.01 mg to 10 mg of peptide, alone or as a mixture, per gram of cream in a white to off-white, opaque cream base of purified water USP, white petrolatum USP, stearyl alcohol NF, propylene glycol USP, polysorbate 60 NF, cetyl alcohol NF, and benzoic acid USP 0.2% as a preservative. In varying embodiments, one or more of the antibacterial peptides can be mixed into a commercially available cream, Vanicream® (Pharmaceutical Specialties, Inc., Rochester, Minn.) comprising purified water, white petrolatum, cetearyl alcohol and ceteareth-20, sorbitol solution, propylene glycol, simethicone, glyceryl monostearate, polyethylene glycol monostearate, sorbic acid and BHT.

In other embodiments, the agent or agents are in a lotion. Typical lotions comprise, for example, water, mineral oil, petrolatum, sorbitol solution, stearic acid, lanolin, lanolin alcohol, cetyl alcohol, glyceryl stearate/PEG-100 stearate, triethanolamine, dimethicone, propylene glycol, microcrystalline wax, tri (PPG-3 myristyl ether) citrate, disodium EDTA, methylparaben, ethylparaben, propylparaben, xanthan gum, butylparaben, and methyldibromo glutaronitrile.

In some embodiments, the peptide or mixtures of peptides are in an oil, such as jojoba oil. In some embodiments, the agent is, or agents are, in an ointment, which may, for example, white petrolatum, hydrophilic petrolatum, anhydrous lanolin, hydrous lanolin, or polyethylene glycol. In some embodiments, the agent is, or agents are, in a spray, which typically comprise an alcohol and a propellant. If absorption through the skin needs to be enhanced, the spray may optionally contain, for example, isopropyl myristate.

In varying embodiments, the peptide or mixture of peptides are administered (that is, whether by lotion, gel, spray, etc.), they are preferably administered at a dosage of about 0.01 mg to 10 mg per 10 cm².

In varying embodiments, the antibacterial peptide or mixture of peptides, can be introduced into the bowel by use of a suppository. As is known in the art, suppositories are solid compositions of various sizes and shapes intended for introduction into body cavities. Typically, the suppository comprises a medication, which is released into the immediate area from the suppository. Typically, suppositories are made using a fatty base, such as cocoa butter, that melts at body temperature, or a water-soluble or miscible base, such as glycerinated gelatin or polyethylene glycol.

The pharmaceutical formulation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the novel unit dosage forms of this invention are dictated by and directly dependent on (a) the unique characteristics of the active material and the particular effect to be achieved and (b) the limitations inherent in the art of compounding such an active material for use in humans and animals, as disclosed in detail in this specification, these being features of the present invention.

In one embodiment, a pharmaceutical formulation is administered to a patient at a therapeutically effective dose to prevent, treat, or control a disease or malignant condition, such as cancer. The pharmaceutical composition or medicament is administered to a patient in an amount sufficient to elicit an effective therapeutic or diagnostic response in the patient. An effective therapeutic or diagnostic response is a response that at least partially arrests or slows the symptoms or complications of the disease or malignant condition. An amount adequate to accomplish this is defined as “therapeutically effective dose.”

4. Subjects Who May Benefit

One or more antibacterial peptides or a composition comprising one or more antibacterial peptides (e.g., a mixture of peptides) can be administered to any subject suffering from or at risk of contracting a bacterial infection to prevent, promote the regression, amelioration and/or mitigation of the bacterial infection and to prevent, reduce and/or inhibit the proliferation and/or growth of the infecting bacteria. In various embodiments, the bacterial infection is a Streptococcus agalactiae, Staphylococcus aureus, Streptococcus uberis, Serratia marcescens, Coagulase-negative staphylococcus (CNS) and/or E. coli infection. The bacterial infection may be local or systemic, as described in further detail below. In varying embodiments, the bacterial infection is treatable by topical administration, is in the oral cavity, on the surface of the skin, in the ear, on the eye and/or conjunctival tissue. For subjects at risk of contracting a bacterial infection, the peptide or peptides are administered to prevent the occurrence or recurrence of the bacterial infection. For subjects who have a bacterial infection or who have been diagnosed with a bacterial infection, the peptide or peptides are administered to promote the regression, amelioration and/or mitigation of the bacterial infection.

In varying embodiments, one or more antibacterial peptides or a composition comprising one or more antibacterial peptides (e.g., a mixture of peptides) are administered to a lactating and/or nursing mother. For the purposes of prevention, the peptide or peptides are administered to prevent the occurrence or recurrence of a bacterial infection, e.g., mastitis. For the purposes of treatment, the peptide or peptides are administered to promote the regression, amelioration and/or mitigation of a bacterial infection, mastitis.

In varying embodiments, one or more antibacterial peptides or a composition comprising one or more antibacterial peptides (e.g., a mixture of peptides) are administered to a nursing infant or child.

In varying embodiments, the subject can be any mammal, e.g., a human, a non-human primate, a domesticated mammal (e.g., canine, feline), an agricultural mammal (e.g., equine, bovine, ovine, porcine), a laboratory mammal (e.g., mouse, rat, rabbit, hamster, guinea pig). In varying embodiments, the subject is a lactating female mammal. In varying embodiments, the subject is a nursing infant mammal.

5. Conditions Subject to Treatment

The antibacterial peptides described herein find use to reduce, inhibit, prevent and/or mitigate a bacterial infection in a subject. In varying embodiments, the bacterial infection is an infection by a bacteria selected from at least one of an aerobic gram-negative bacteria, aerobic gram-positive bacteria, and anaerobic gram-negative bacteria. In varying embodiments, the bacterial infection may comprise more than one of an aerobic gram-negative bacteria, aerobic gram-positive bacteria, and anaerobic gram-negative bacteria.

In some embodiments, the subject has an infection of gram-positive bacteria, e.g., Streptococcus, Staphylococcus, Enterococcus, Gram positive cocci, and Peptostreptococcus. In some embodiments, the gram-positive bacteria is selected from beta-hemolytic Streptococcus, coagulase negative Staphylococcus, Enterococcus faecalis (VSE), Staphylococcus aureus, and Streptococcus pyogenes. In some embodiments, the gram-positive bacteria is selected from methicillin-sensitive Staphylococcus aureus (MSSA), and methicillin-resistant Staphylococcus aureus (MRSA).

In some embodiments, the subject gram-negative bacteria is selected from Acinetobacter, Alcaligenes, Bacteroides, Burkholderia, Enterobacter, Klebsiella, Morganella, Ochrobactrum, Proteus, Providencia, Pseudomonas, and Serratia. In some embodiments, the gram-negative bacteria is selected from Alcaligenes faecalis, Bacteroides fragilis, Escherichia coli, Enterobacter cloacae, Klebsiella oxytoca, Morganella morganii, Ochrobactrum anthropi, Providencia rettgeri, Pseudomonas aeruginosa, and Serratia marcescens.

In varying embodiments, the bacterial infection is selected from a soft tissue bacterial infection, a hard tissue bacterial infection, or a combination thereof. In some embodiments, the bacterial infection is a hard tissue bacterial infection, for example, osteomyelitis.

In varying embodiments, the antibacterial peptides find use in treating infected ulcers, e.g., infected diabetic ulcers, comprising administration of the peptide or a mixture of peptides. In varying embodiments, the peptide or mixture of peptides are administered topically, e.g., at the site of infection. In varying embodiments, the ulcer is a diabetic ulcer, e.g., a diabetic lower limb ulcer or a diabetic foot ulcer.

In some embodiments, the bacterial infection is a bacterial infection of a wound, e.g., from venous stasis ulcers, arterial ulcers, decubitus ulcers, surgical wounds, radiation ulcers, and wounds caused by a burn.

In varying embodiments, the antibacterial peptides described herein are useful for the treatment of an infection of the mammary gland. For example, the antibacterial peptides are useful in treating mastitis, in humans and in non-human mammals, including livestock animals, e.g., cows, sheep, buffalos and goats.

Clinical and subclinical mastitis are inflammatory states of the udder resulting mainly from bacterial infection. Mastitis has a variety of bacterial etiologies and causes great losses in milk production annually. Pathogenic microorganisms that most frequently cause mastitis can be divided into two groups based on their source: environmental pathogens and contagious pathogens. The major contagious pathogens are Streptococcus agalactiae, Staphylococcus aureus, Coagulase-negative staphylococcus (CNS) and E. Coli. With the exception of some mycoplasmal infections that may originate in other body sites and spread systemically, these microorganisms gain entrance into the mammary gland through the teat canal. Contagious organisms are well adapted to survival and growth in the mammary gland and frequently cause infections lasting weeks, months or years. The infected gland is the main source of these organisms, e.g., in a dairy herd and transmission of contagious pathogens to uninfected quarters and cows occurs mainly during milking time.

Clinical mastitis is easily diagnosed due to marked alterations in milk composition and appearance, decreased milk production, elevated body temperature and swelling, redness, or fever in the infected glands. Subclinical mastitis, the most prevalent form of the disease, often remains undetected because signs are not readily apparent. Many subclinical intramammary infection (IMI) tend to persist, resulting in a decrease of milk quality due to elevated milk somatic cell count (SCC), and also due to a decrease in milk production. IMI localized in a single mammary gland may lead to the development of clinical mastitis and to the spread of certain mastitis pathogens from infected mammary quarters to uninfected ones. In contrast to clinical mastitis, it is not usually advisable to treat livestock animals having subclinical mastitis by antibiotic administration during lactation (Gruet et al., 2001. Adv. Drug Delivery Rev. 50:245-259) because the cure rate is low and because the cost of the treatment and a withdrawal period of 4-5 days of milk make it economically unjustified (Yamagata et al., 1987. J. Am. Vet. Med. Assoc. 191:1556-1561). The pharmaceutical compositions of the present invention can be administered during the lactating period. As described herein, the compositions of the invention can have a local effect, such that the treatment can be administered only to the infected mammary gland(s), while milking from the uninfected gland(s) can continue, reducing the milk loss to a minimum.

For treating mastitis, administration of repeated doses of the pharmaceutical compositions of the invention into the infected mammary gland may be required. In varying embodiments, administration is repeated at least once, preferably between 1-10 times, more preferably 1 to 3 times, at an interval selected from the group consisting of about 6 hours, about 8 hours, about 12 hours, about 16 hours, about 20 hours and about 24 hours during 1 to 10 days, preferably 1 to 3 days.

In varying embodiments, the antibacterial peptides are administered in combination with an additional anti-microbial treatment selected from the group consisting of, but not limited to, antibiotic, bactericide, steroidal and non-steroidal anti-inflammatory treatment, treatment with an immunomodulator and vaccination. According to one embodiment, the pharmaceutical composition of the present invention and the additional anti-microbial treatment are co-administered, either as a combined, single pharmaceutical composition or as separate compositions. Alternatively, the pharmaceutical composition of the present invention is administered as a pre-treatment followed by the application of the additional anti-microbial treatment, and vice-versa.

6. Methods of Monitoring

A variety of methods can be employed in determining efficacy of therapeutic and prophylactic treatment with the antibacterial peptides of the present invention. Generally, efficacy is the capacity to produce an effect without significant toxicity. In varying embodiments, efficacy can be measured by comparing treated to untreated individuals or by comparing the same individual before and after treatment. Efficacy of a treatment can be determined using a variety of methods, including pharmacological studies, diagnostic studies, predictive studies and prognostic studies. Examples of indicators of efficacy include but are not limited to inhibition and or regression of bacterial cell growth, bacterial cell burden, inflammation, swelling, lesions and other symptoms associated with bacterial infection (e.g., fatigue, malaise, nausea) and promotion of healing and bacterial death.

The efficacy of administration of the anti-bacterial peptides can be assessed by a variety of methods known in the art. Administration of one or more antibacterial peptides, described herein, can be screened for prophylactic or therapeutic efficacy in animal models in comparison with untreated or placebo controls. The one or more antibacterial peptides can be then analyzed for the capacity to promote bacterial cell death or enhanced regression or reversal or bacterial cell infection. For example, multiple dilutions of an infected biological sample (e.g., blood, serum, plasma, milk, urine, mucous, saliva or cerebrospinal fluid) can be tested for examining bacterial cell burden and/or growth. Standard protocols are known in the art. See, e.g., Green and Sambrook, Molecular Cloning: A Laboratory Manual (Fourth Edition), Cold Spring Harbor Laboratory Press, 2012; Ausubel, et al. Editor, Current Protocols in Molecular Biology, USA, 1984-2012; Bonifacino, et al., Editor, Current Protocols in Cell Biology, USA, 2010; all of which are incorporated herein by reference in their entirety.

The methods provide for detecting prevention, inhibition and/or reversal of bacterial infection in patients suffering from or susceptible to bacterial infection. A variety of methods can be used to monitor both therapeutic treatment for symptomatic patients and prophylactic treatment for asymptomatic patients.

Monitoring methods entail determining a baseline value of a bacterial burden, milk somatic cell counts (SCC) and/or symptoms (e.g., pain, swelling, tenderness, inflammation, lesions, fatigue, malaise, nausea) in a patient before administering a dosage of one or more of the antibacterial peptides, and comparing this with a value for the bacterial burden and/or symptoms after treatment, respectively.

With respect to therapies administering one or more of the antibacterial peptides, a significant decrease (i.e., greater than the typical margin of experimental error in repeat measurements of the same sample, expressed as one standard deviation from the mean of such measurements) in value of the bacterial cell burden signals a positive treatment outcome (i.e., that administration of the one or more antibacterial peptides has reversed, inhibited, or reduced progression of bacterial growth and/or infection).

In other methods, a control value of bacterial cell burden (e.g., a mean and standard deviation) is determined from a control population of individuals who have undergone treatment with one or more of the antibacterial peptides. Measured values of bacterial cell burden in a patient are compared with the control value. If the measured level in a patient is not significantly different (e.g., more than one standard deviation) from the control value, treatment can be discontinued. If the bacterial cell burden level in a patient is significantly above the control value, continued administration of agent is warranted.

In other methods, a patient who is not presently receiving treatment but has undergone a previous course of treatment is monitored for bacterial cell burden to determine whether a resumption of treatment is required. The measured value of bacterial cell burden in the patient can be compared with a value of bacterial cell burden previously achieved in the patient after a previous course of treatment. A significant increase in bacterial cell burden relative to the previous measurement (i.e., greater than a typical margin of error in repeat measurements of the same sample) is an indication that treatment should be resumed. A significant decrease in bacterial cell burden relative to the previous measurement (i.e., greater than a typical margin of error in repeat measurements of the same sample) is an indication that treatment need not be resumed. Alternatively, the value measured in a patient can be compared with a control value (mean plus standard deviation) determined in a population of patients after undergoing a course of treatment. Alternatively, the measured value in a patient can be compared with a control value in populations of prophylactically treated patients who remain free of symptoms of infection, or populations of therapeutically treated patients who show amelioration of disease characteristics. In all of these cases, a significant increase in bacterial cell burden relative to the control level (i.e., more than a standard deviation) is an indicator that treatment should be resumed in a patient.

The tissue sample for analysis is typically blood, plasma, serum, mucous, milk, saliva, urine or cerebrospinal fluid from the patient. The sample can be analyzed for indication of bacterial cell infection. Bacterial cell burden can be detected using any method known in the art, e.g., visual observation of a tissue sample by a qualified pathologist, or other techniques (e.g., amplification of a nucleic acid specific to and indicative of the bacteria, bacterial culture).

### EXAMPLES

The following examples are offered to illustrate, but not to limit the claimed invention.

### Example 1

### Inhibition of S. Aureus Growth by Naturally-Occurring Peptides from Human and Bovine Milk

### Materials and Methods:

Milk Peptide Isolation.

Milk fat fractionation of the samples was performed by centrifugation at 15,000 rpm for 10 min at 4° C. The skim milk infranate was removed from beneath the fat layer by pipette. The procedure was repeated until no fat was observed.

Proteins were precipitated by adding 1:1 (v/v) of 200 g/L trichloroacetic acid in nanopure water to the skim milk. The samples were mixed using a vortex mixer, centrifuged at 3,000×g at 4° C. for 10 min and the supernatant was collected.

Solid phase extraction was performed with C18 columns (Supelco) in order to remove contaminants. The peptides were eluted using an 80% acetonitrile, 1% trifluoroacetic acid solution. Samples were finally dried down and rehydrated in nanopure water for the bacterial assay.

Bacterial Growth Assay.

The milk peptides were tested for antimicrobial activity against S. aureus. The experiments were performed in triplicate using different number of bacteria for the inoculation.

The underlay medium used for the bacteria growth is composed by diluted trypticase soy broth (TSB) 30 mg, 1% (w/v) agarose and 2 mL of 10% Tween-20 in 10 mM phosphate buffer. Different amounts of bacteria were inoculated in this medium. The medium was poured onto plates and left to solidify. Once the agarose solidified, 3 mm holes were punched in the plate. Holes B2, B4 and C3 were loaded with 4 μL, of the bovine milk peptide mixture at different concentrations (10 μg/μL, 6 μg/μL and 3 μg/μL, respectively). Well C5 and D2 were loaded with 4 μL, of the human milk peptide mixture at different concentrations (8 μg/μL and 4 μg/μL, respectively) F2 was loaded with 1 μg/μL maganinan—antimicrobial peptide—as the positive control. Well F4 was loaded with 1 μg/μL human defensin-6 as the negative control. Well E3 was loaded with nanopure water as another negative control. Then, the plates were incubated for 3 h at 37° C. The overlay medium—composed of 6 g TSB, 1% (w/v) agarose in 10 mM phosphate buffer—was added to the top of each plate. After solidifying, the plates were incubated overnight at 37° C.

### Results

All three plates clearly show that the S. aureus bacterial growth was inhibited by both peptide mixtures. The results are shown in FIGS. 1A-C.

### Conclusion

Peptides isolated from human and bovine milk inhibited the growth of S. aureus.

### Example 2

### Inhibition of E. coli Growth by Naturally-Occurring Peptides from Human Milk

### Materials and Methods:

Milk Peptide Isolation.

Milk fat fractionation of the sample was performed by centrifugation at 15,000 rpm for 10 min at 4° C. The skim milk infranate was removed from beneath the fat layer by pipette. The procedure was repeated until no fat was observed.

Proteins were precipitated by adding 1:1 (v/v) of 200 g/L trichloroacetic acid in nanopure water to the skim milk. The samples were mixed using a vortex mixer, centrifuged at 3,000×g at 4° C. for 10 min and the supernatant was collected.

Solid phase extraction was performed with C18 columns (Supelco) in order to remove contaminants. The peptides were eluted using an 80% acetonitrile, 1% trifluoroacetic acid solution. Samples were finally dried down and rehydrated in nanopure water for the bacterial assay.

Bacterial Growth Assay.

The milk peptides were then tested for antimicrobial activity against E. coli, strain D31. The experiments were performed in triplicate using different number of bacteria for the inoculation.

The underlay medium used for the bacteria growth is composed by diluted trypticase soy broth (TSB) 30 mg, 1% (w/v) agarose and 2 mL of 10% Tween-20 in 10 mM phosphate buffer. Different amounts of bacteria were inoculated in this medium. The medium was poured onto plates and left to solidify. Once the agarose solidified, 3 mm holes were punched in the plate. Holes B2, B4, C3 and C5 were loaded with 4 μL of the peptide mixture at different concentrations (6 μg/μL, 0.6 μg/μL, 0.06 μg/μL and 0.006 μg/μL, respectively). Well F1 was loaded with 1 μg/μL maganinan—antimicrobial peptide—as the positive control. Well F4 was loaded with 1 μg/μL human defensin-6 as the negative control. Well D6 was loaded with nanopure water as another negative control. Then, the plates were incubated for 3 h at 37° C. The overlay medium—composed of 6 g TSB, 1% (w/v) agarose in 10 mM phosphate buffer—was added to the top of the plates. After solidifying, the plates were incubated overnight at 37° C.

### Results

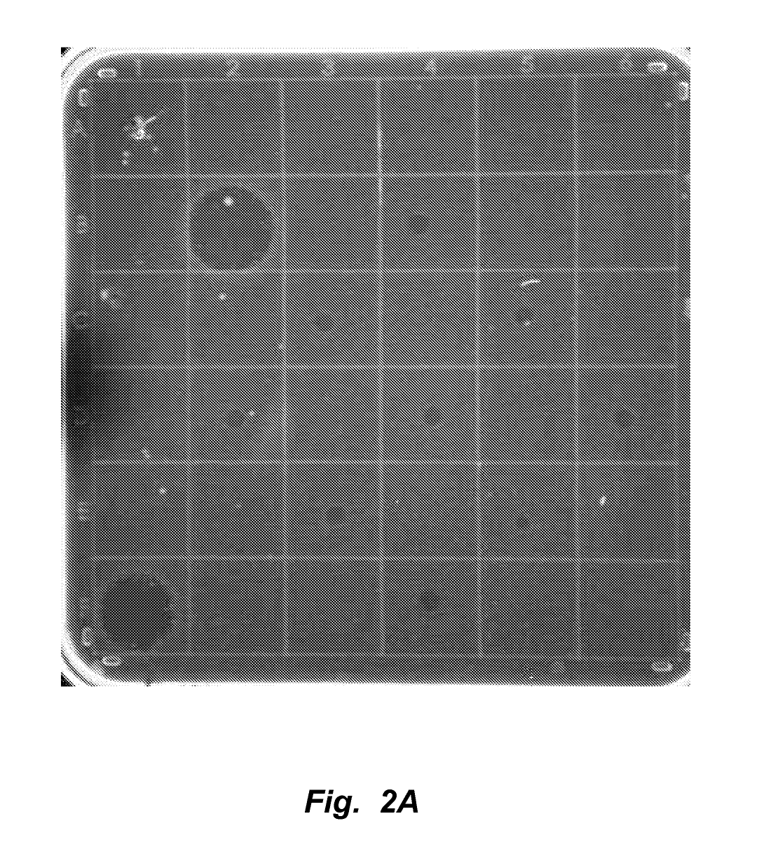

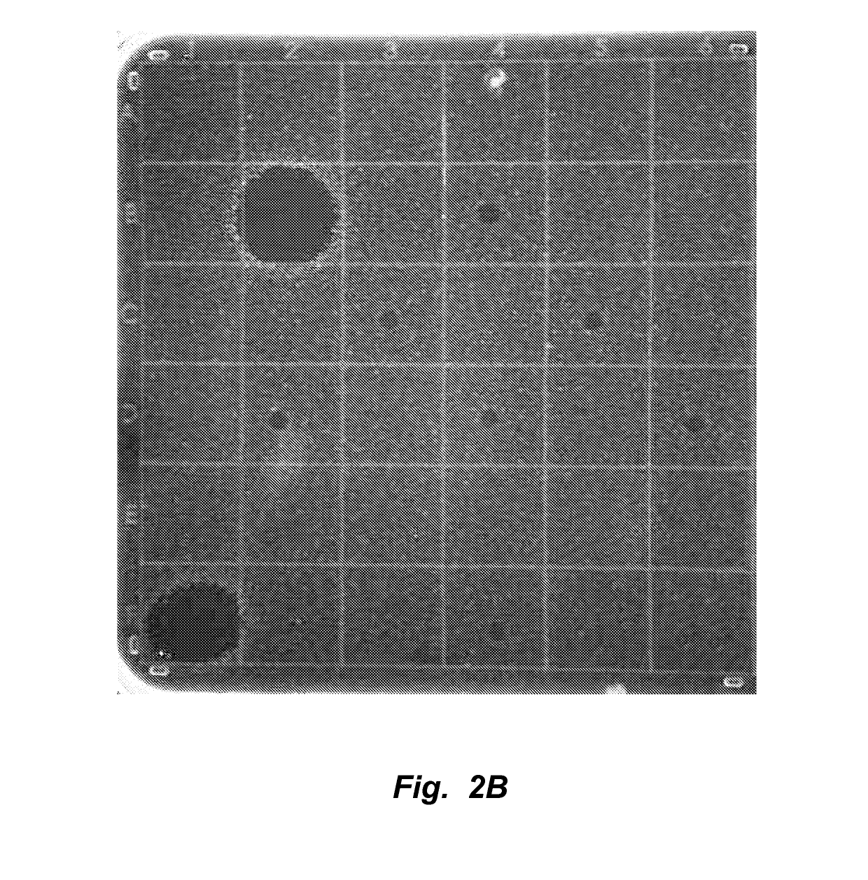

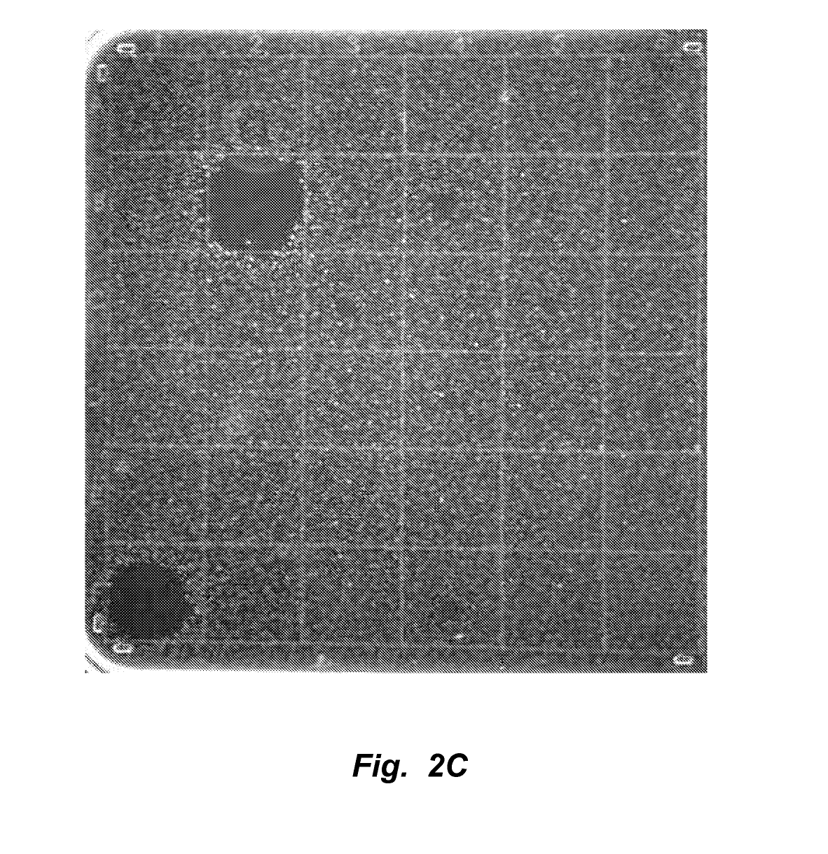

All three plates clearly show that E. coli bacterial growth was inhibited by the 6 μg/μL concentration of milk peptides. The results are shown in FIGS. 2A-C.

### Conclusion

Peptides isolated from human milk inhibited the growth of E. coli.

### Example 3

### Naturally-Occurring Peptides in Human Milk: Identification and Evidence for Antibacterial Action

### Materials and Methods

Chemicals and Sample Set.

Acetonitrile (ACN), formic acid (FA) and trifluoroacetic acid (TFA) were obtained from Thermo Fisher Scientific (Waltham, Mass.) and trichloroacetic acid (TCA) from EMD Millipore (Darmstadt, Germany). Insulin chain A from bovine pancreas was obtained from Sigma-Aldrich (St. Louis, Mo.).

Milk samples from two mothers who delivered at term were pooled for this study. Both milk samples were mature (from three months of lactation). Both donors were healthy and gave birth to healthy infants. Milk samples were taken from milk expressed by breast milk pumps, transferred into sterile plastic containers and immediately stored in home freezers. Manual expression typically takes 10-15 min during which milk samples were exposed to room temperature. Milk samples were transported on dry ice to the laboratory where they were stored at 80° C. until the moment of the sample preparation.

Sample Preparation.

Milk fat fractionation of the sample was performed according to method described by Dallas et al. (Dallas, et al., J Agr Food Chem (2011) 59(8):4255-4263). Briefly, 500 μL, of the pooled sample was centrifuged at 16,000×g for 10 min at 4° C. and the skim milk infranate was removed from beneath the fat layer by pipette. The procedure was repeated until no fat was observed.

Proteins were removed by five different procedures for comparison to determine the method that captures the highest amount of peptides with the least amount of large protein contamination. TCA precipitation: Peptides were precipitated according to the method of Ferranti et al. (Ferranti, et al., J. Dairy Res. (2004) 71(1):74-87). Briefly, 300 μL of 200 g/L TCA in nanopure water were added to 300 μL of skim milk. The samples were mixed using a vortex mixer, centrifuged at 3,000×g at 4° C. for 10 min and the supernatant was collected. Acetonitrile precipitation: Acetonitrile precipitation was performed according to Merrell et al. (J Biomol Tech. (2004) 15(4):238-48). Briefly, 600 μL of ACN were added to the 300 μL sample and vortexed briefly. The sample was then incubated at room temperature for 30 min and centrifuged at 12,000 rpm for 10 min at room temperature. The supernatant was collected, dried down and reconstituted in water. Acetone precipitation: Acetone precipitation was performed according to a Pierce Biotech protocol (on the internet at bidmcmassspec.org/uploads/Acetone_precipitation.pdf). Briefly, 4 volumes of −20° C. acetone were added to the sample. After vortexing, the sample was placed at −20° C. for 1 h. Finally, the sample was centrifuged for 10 min at 14,000×g at room temperature and the supernatant was collected, dried down and reconstituted in water. The fractions obtained from these three procedures were cleaned of contaminants, mainly oligosaccharides, through solid phase extraction (SPE) with 500 mg bed C18 columns (Supelco). The peptides were eluted from the column using 80% ACN, 0.1% TFA solution.

C18 only: Peptide isolation was performed only by running skim milk on a C18 column according to the method above. C8 only: Peptide isolation was performed only by running on a 500 mg bed C8 column (Supelco) according to the method above. All the samples were finally dried down.

Peptide/Protein Content Estimation.

To determine the effectiveness of the various peptide isolation techniques, peptide concentration was determined by measuring absorbance at 205 nm 33 with an IMPLEN P300 nano spectrophotometer. For determination of protein concentration, 280 nm is usually the wavelength of choice, corresponding to an absorbance maximum of the aromatic rings of the amino acids tryptophan, tyrosine and phenylalanine 1n our case, due to the small size of the peptides, not all contain aromatic amino acids and therefore 205 nm, corresponding to a maximum absorbance of the peptidic bond, was used. Briefly, a standard concentration curve was created with insulin chain A peptide (Sigma). Then, samples were hydrated in 100 μL of nanopure water and peptide concentration was measured with 2 μL of sample.

In addition to the absorbance measurements, each sample was run on a 1-dimensional 12% acrylamide Mini-Protean TGX gel (BioRad) to determine the amount of large, intact protein that remained in the peptide sample after isolation. Each lane was run with roughly 50 μg or 10 μg of protein. Samples were mixed 1:1 with Laemmli buffer, then mixed with 1:10 1 M dithiothreitol:sample and boiled for 1 min. Then, samples were mixed with 1:10 100 mM iodoacetamide and incubated in darkness at room temperature for 30 min. The gels were run for 1 h at 140 V. After running, the gels were soaked in water for 15 min, then soaked in Coomassie stain for 2 h and finally soaked in water overnight.

Mass Spectrometry Analysis.

Samples were rehydrated with 40 μA of nanopure water prior to mass spectrometry analysis. Samples (2 μL/injection) were analyzed on an Agilent (Santa Clara, Calif.) nano-LC-chip-Q-TOF MS/MS (Chip-Q-TOF) with an Agilent chip C18 column at a flow rate of 0.3 4/min. The gradient elution solvents were (A) 3% ACN/0.1% formic acid (FA) and (B) 90% ACN/0.1% FA. The gradient employed was ramped from 0-8% B from 0-5 min, 8-26.5% B from 5-24 min, 26.5-100% B from 24-48 min, followed by 100% B for 2 min and 100% A for 10 min (to re-equilibrate the column). The capillary pump was set to 3.5 4/min and 0% B throughout the analysis. Ion polarity was set to positive. The peak collection thresholds were set at 200 ion counts or 0.01% relative intensity for MS spectra and 5 ion counts or 0.01% relative intensity for MS/MS. Data were collected in centroid mode. The drying gas was 350° C. and flow rate was 3 L/min. The required chip voltage for consistent spray varied from 1850 to 1920 V. Automated precursor selection based on abundance was employed to select peaks for tandem fragmentation with an exclusion list consisting of all peptides identified in previous analyses in this study. The acquisition rate employed was 3 spectra/s for both MS and MS/MS modes. The isolation width for tandem analysis was 1.3 m/z. The collision energy was set by the formula (Slope)*(m/z)/100+Offset, with slope=3.6 and offset=−4.8. Five tandem spectra were collected after each MS spectrum, with active exclusion after 5 MS/MS for 0.15 min. Precursor ions were only selected if they had at least 1000 ion counts or 0.01% of the relative intensity of the spectra. Mass calibration was performed during data acquisition based on an infused calibrant ion with a mass of 922.009789 Da.

Data Analysis.

Agilent Mass Hunter Qualitative Analysis Software (Santa Clara, Calif.) was used to analyze the data obtained. Molecules identified in the spectral analysis were grouped into compounds by the Find by Molecular Feature algorithm, which groups together molecules across charge state and charge carrier. All tandem-MS from each data file were exported as Mascot Generic Files (.mgf) with a peptide isotope model and a maximum charge state of +9.

Peptide identification was accomplished using both the MS-GFDB (via a command-line interface) and X!Tandem (using the downloadable graphical user interface). The human milk library used in both searches was constructed based on a query to the Uniprot database. The query returned only proteins from Homo sapiens and at least one of the following: “tissue specificity” keyword “milk” or “mammary”, “tissue” keyword “milk” or “mammary” or gene ontology “lactation”. This query returned a list of 1,472 proteins. These were exported to FASTA file format. For MS-GFDB, peptides were accepted if p-values were less than or equal to 0.05 and 0.01 corresponding to confidence levels of 95% and 99% respectively. No p-values exist in X!Tandem, so a closely related statistic, e-value, was used for the X!Tandem search. The e-value thresholds selected were again 0.05 and 0.01. In both programs, masses were allowed 20 ppm error. No complete (required) modifications were included but up to four potential modifications were allowed on each peptide. Potential modifications allowed were phosphorylation of serine, threonine or tyrosine and oxidation of methionine. A non-specific cleavage ([X]|[X]) (where ‘X’ is any amino acid) was used to search against the protein sequences. For MS-GFDB, the fragmentation method selected in the search was CID and the instrument selected was TOF. For X!Tandem, there was no option for fragmentation type and instrument selection. Because the instrument did not always select the monoisotopic ion for tandem fragmentation, isotope errors were allowed (allowing up to one C13). No model refinement was employed in X!Tandem.

Exclusion List Creation.

After each analysis, newly-identified peptides were added to an in-house database for the sample. This database was used to create an exclusion list, composed of mass-to-charge signals, charge state and their corresponding retention times, for further tandem analysis. Molecular ions on the exclusion list were ignored by the instrument and hence were not fragmented again. This approach allowed deeper exploration of the data, namely, identification of peaks at low abundance. A +/−20 ppm error window was employed. The retention time window was set at +/−0.5 min. For the sixth analysis, the exclusion list incorporated all masses fragmented in the fifth analysis, as many of these peaks had been fragmented many times without successful identification. Placing these peaks on the exclusion list allowed the instrument to fragment peaks of lesser abundance that co-eluted with these unidentified compounds. Inclusion of all fragmented molecules in the exclusion list (including non-identified signals) was repeated for analyses 13, 15, 16, 17, 18 and 19.

Search for Known Bioactive Peptides.

To uncover breast milk peptides that overlap with existing bioactive peptides in the literature, identified peptides were compared to sequences from four bioactive peptide databases: BIOPEP (Dziuba, et al., Food/Nahrung (1999) 43(3):190-195), PeptideDB (Liu, et al, Journal of Proteome Research (2008) 7(9):4119-4131), CAMP (Thomas, et al., Nucleic Acids Research (2010) 38(suppl 1):D774-D780), and APD2 (Wang, et al., Nucleic Acids Research (2009) 37(suppl 1):D933-D937). We merged all four databases and parsed this dataset to remove duplicates. Because hormone peptides in these databases could be very large, the new database was restricted to hormonal peptides less than 60 amino acids in length.

Each breast milk peptide was searched against the database using protein-protein BLAST (BLASTP). For each query, a known bioactive peptide was retained if E-values were less than 0.5 and at least 50% of the query sequence was covered by the library sequence. This high E-value was chosen to counter-balance the effect of the small size of the milk peptides, which as an effect will have higher E-values. The high E-value threshold allowed for discovery of overlapping sequences that would be missed with a smaller E-value threshold. The BLASTP output was parsed to remove false positives.

Antimicrobial Assays.

For the antimicrobial assays, peptides were obtained from the TCA precipitation method for peptide isolation. These peptides were tested for antimicrobial activity against Escherichia coli (E. coli), strain D31 and Staphylococcus aureus (S. aureus). The experiments were performed in triplicate, using different numbers of bacteria for the plate inoculation. The underlay medium used for the bacteria growth is composed by diluted trypticase soy broth (TSB) 30 mg, 1% (w/v) agarose and 2 mL of 10% Tween-20 in 10 mM phosphate buffer. Bacteria were inoculated in this medium at the following concentrations: 10⁴, 10⁵ and 10⁶ bacteria. The medium was poured onto plates and left to solidify. Once the agarose solidified, 3 mm holes were punched in the plate. On the E. coli plate, holes B2, B4, C3 and C5 were loaded with 4 μL of the peptide mixture at different concentrations (6 μg/μL, 0.6 μg/μL, 0.06 μg/μL and 0.006 μg/μL, respectively), well F1 was loaded with 1 μg/μL maganinan antimicrobial peptide—as the positive control, well F4 was loaded with 1 μg/μL human defensin-6 as the negative control, and well D6 was loaded with nanopure water as another negative control. For the S. aureus assay, wells C5 and D2 were loaded with 4 μL of the human milk peptide mixture at different concentrations (8 μg/μL and 4 μg/μL, respectively), F2 was loaded with 1 μg/μL maganin, well F4 was loaded with 1 μg/μL human defensin-6, and well E3 was loaded with nanopure water. Then, the plates were incubated for 3 h at 37° C. The overlay medium—composed of 6 g TSB, 1% (w/v) agarose in 10 mM phosphate buffer—was added to the top of the plates. After solidifying, the plates were incubated overnight at 37° C. Expansion of areas with no bacterial growth around the well demonstrates inhibition of bacterial growth from the compound in that well.

### Results and Discussion

Peptide Isolation Technique Comparison.

The goal of this peptide isolation was to remove all intact proteins and isolate as much small peptide fragment material as possible. From the six peptide isolation techniques compared, “C8 only” isolated the highest concentration of peptides/proteins, whereas acetone precipitation isolated the least (see Table 2).

TABLE 2
Peptide concentration after each peptide isolation technique.

| | ACN | Acetone | TCA | C18 | C8 |
|---|---|---|---|---|---|
| Isolation Method | ppt. | ppt. | ppt. | only | only |
| Yield (mg/mL of milk) | 3.11 | .098 | .35 | 5.7 | 8.9 |

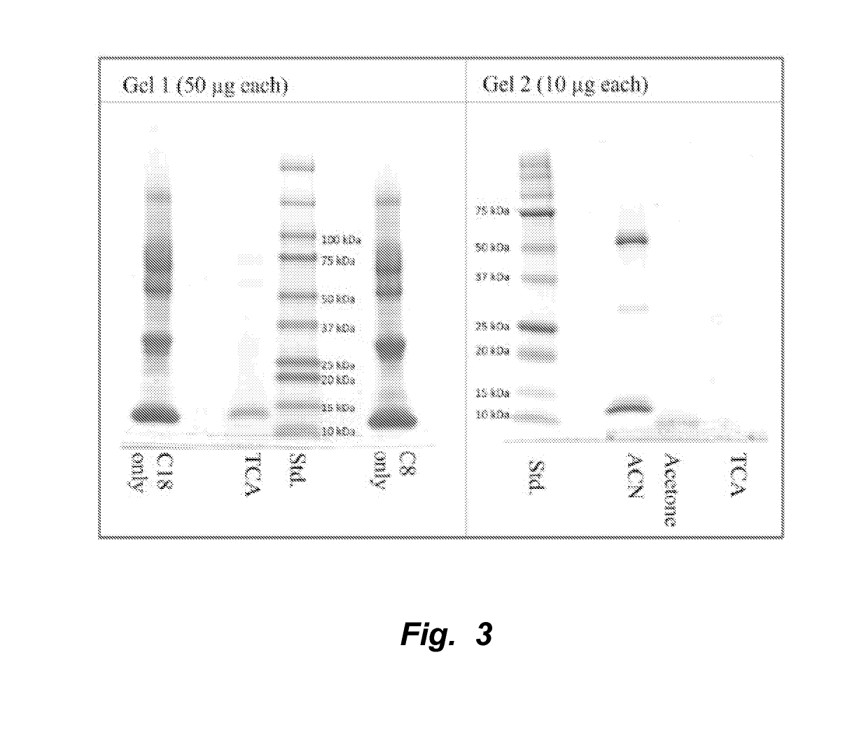

The gels run to determine the presence of large intact proteins (FIG. 3) show that “C18 only” and “C8 only” performed most poorly for intact protein elimination. ACN precipitation performed better that “C8” or “C18 only,” but still a few large and prominent protein bands remained. Both TCA precipitation and acetone precipitation showed nearly complete large protein removal. The only proteins present in the TCA precipitation sample was a band between 10-15 kDa. For our purposes, an isolation of peptides and proteins <15 kDa is adequate. Though acetone precipitation also eliminated all protein bands, we selected TCA precipitation for the rest of our experimental work because the protein/peptide yield for TCA precipitation was over 6 times greater than that for acetone precipitation.

Peptide Identification.

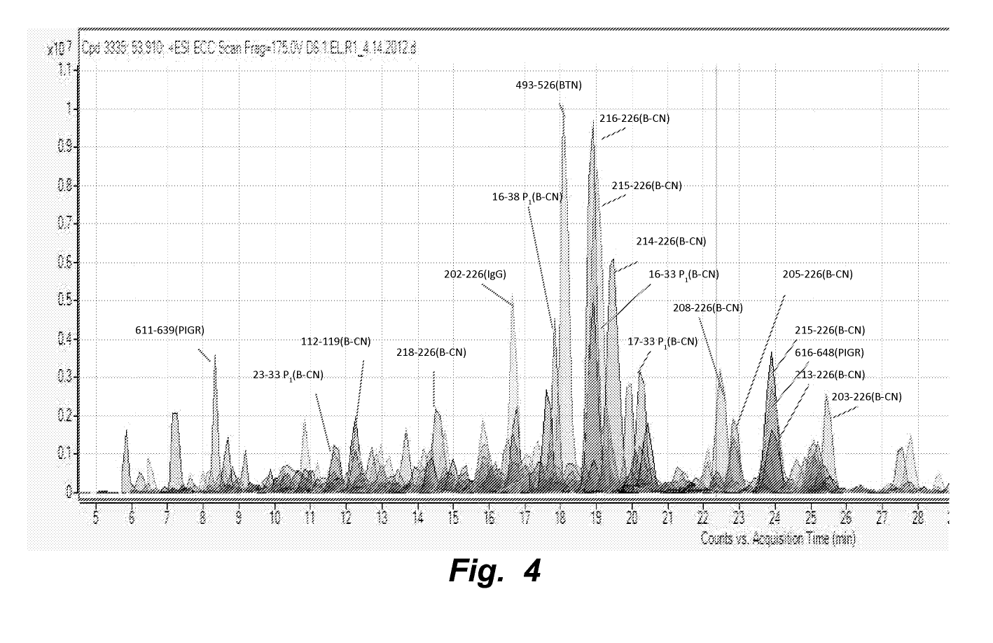

FIG. 4 shows part of the Extracted Compound Chromatograms (ECC) from the first mass spectrometry analysis. After identification, peptides were matched to the compounds they represent in the chromatogram. This chromatogram shows that peptides were separated by retention time and by mass in a bidimensional separation.

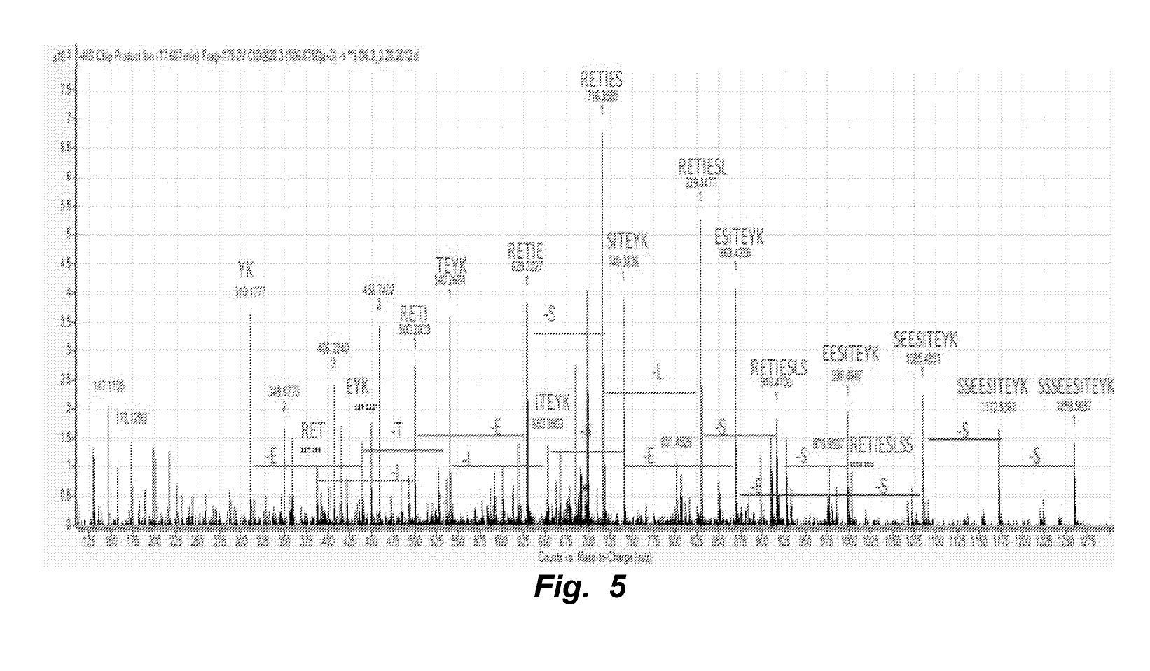

Peptides were identified in X!Tandem and MS-GFDB with a database search of the MS/MS spectra (FIG. 5). For each intact mass, these databases matched the fragments present in the tandem spectra against predicted fragments for peptide sequences matching the intact mass.

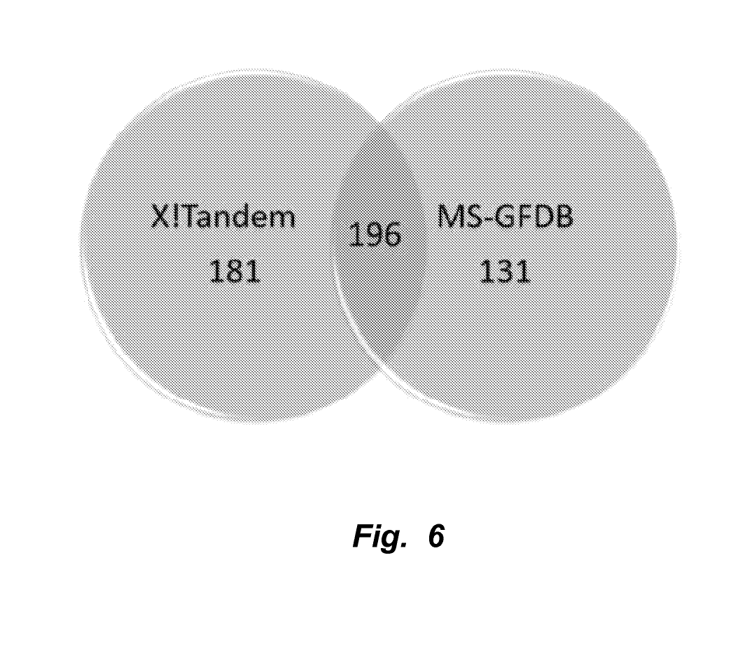

A perfect comparison of X!Tandem and MS-GFDB results was not possible because X!Tandem reports peptide e-values and not p-values and MS-GFDB reports peptide p-values and not e-values. Instead, both e-values and p-values were employed with a 0.01 threshold for both. FIG. 6 shows a Venn diagram of the unique peptides identified in each program. Thirty-nine percent of the unique peptides were found in both programs and X!Tandem identified approximately 10% more peptides than MS-GFDB.

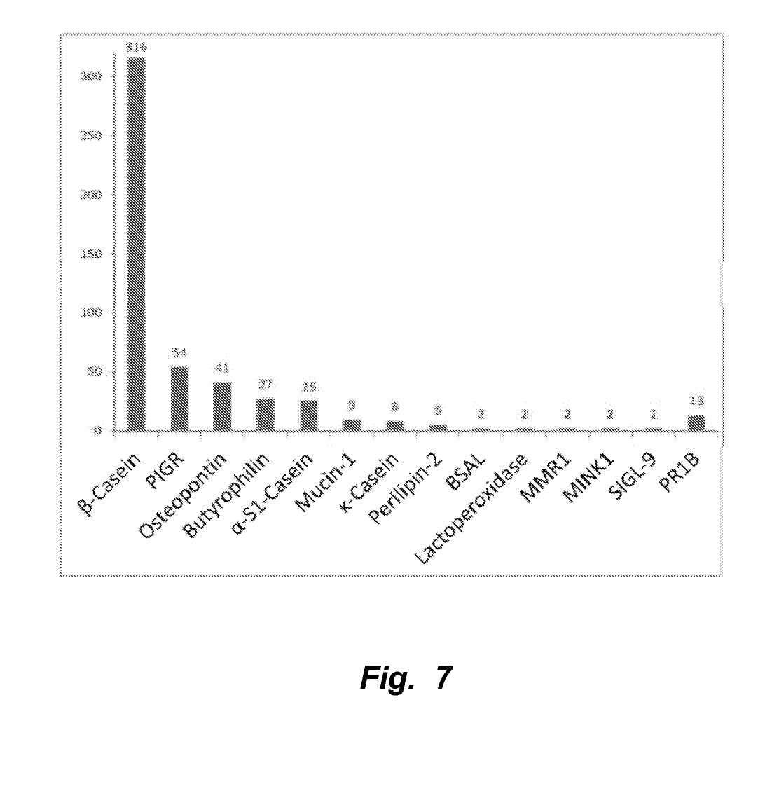

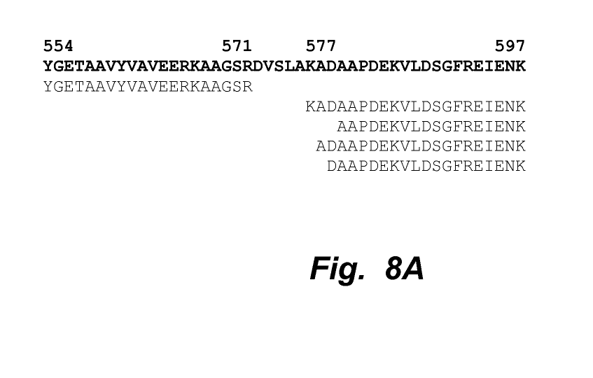

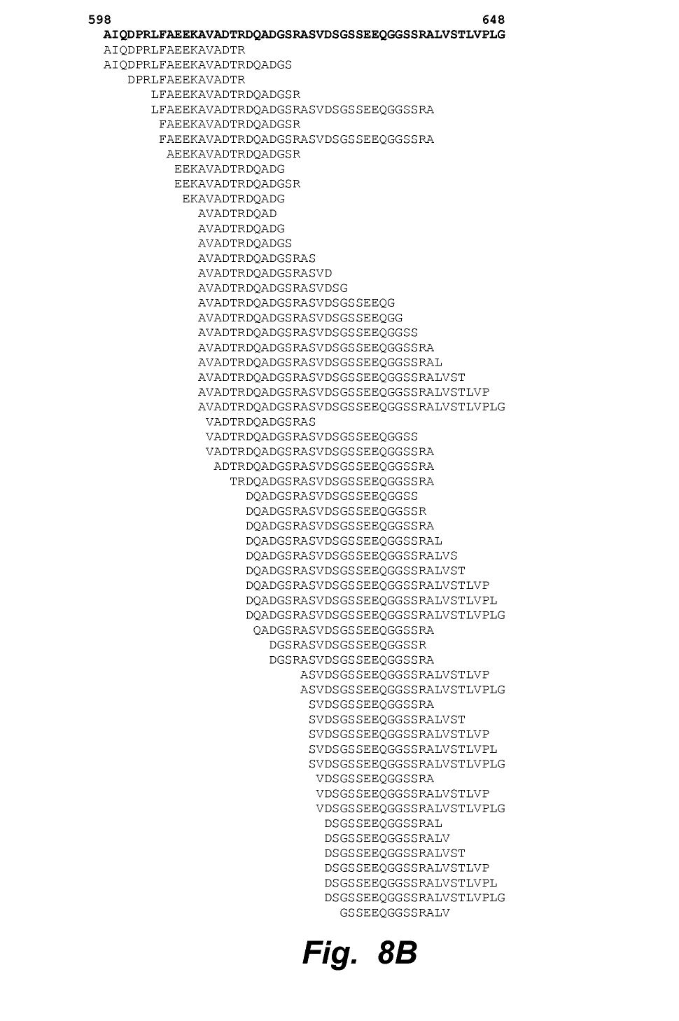

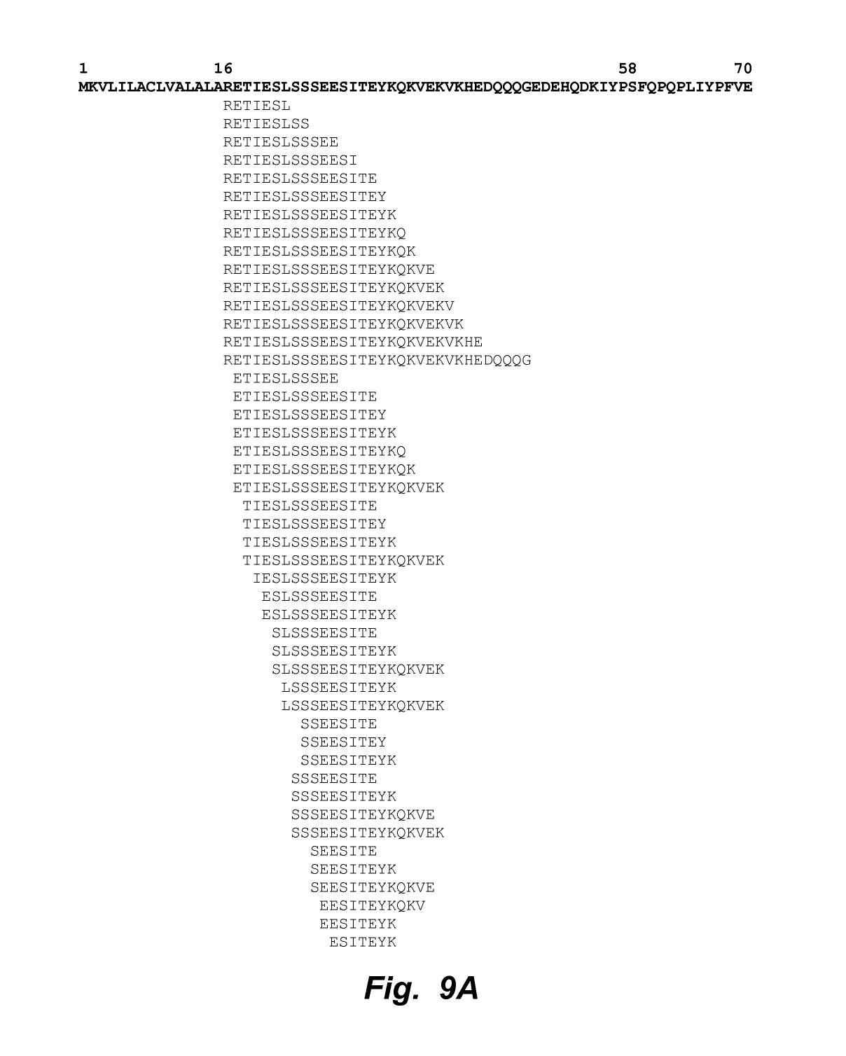

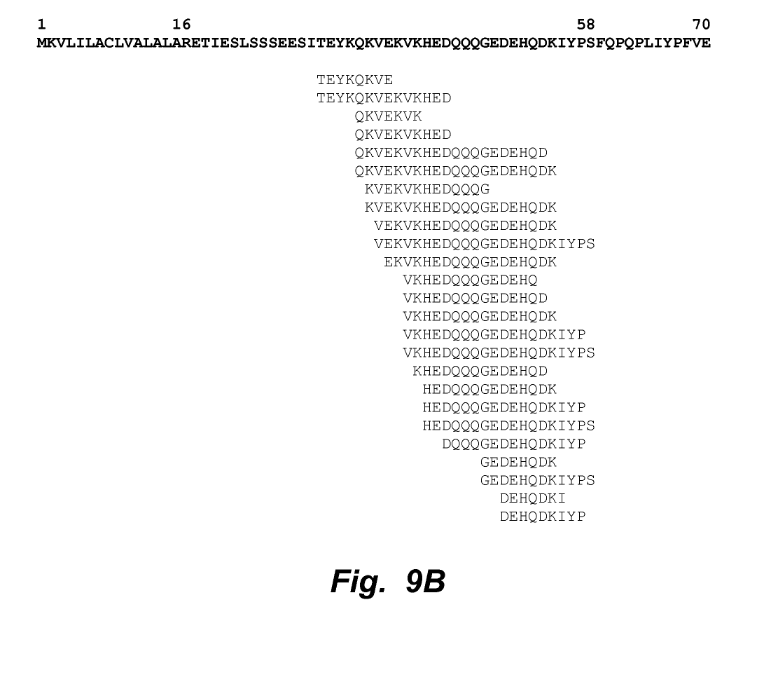

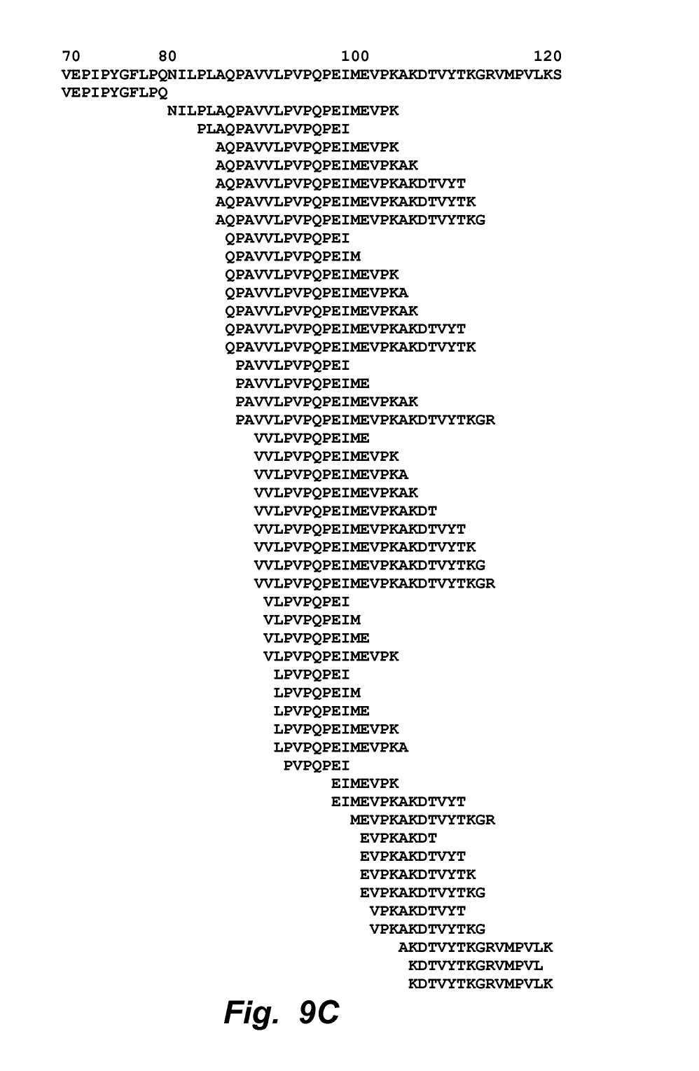

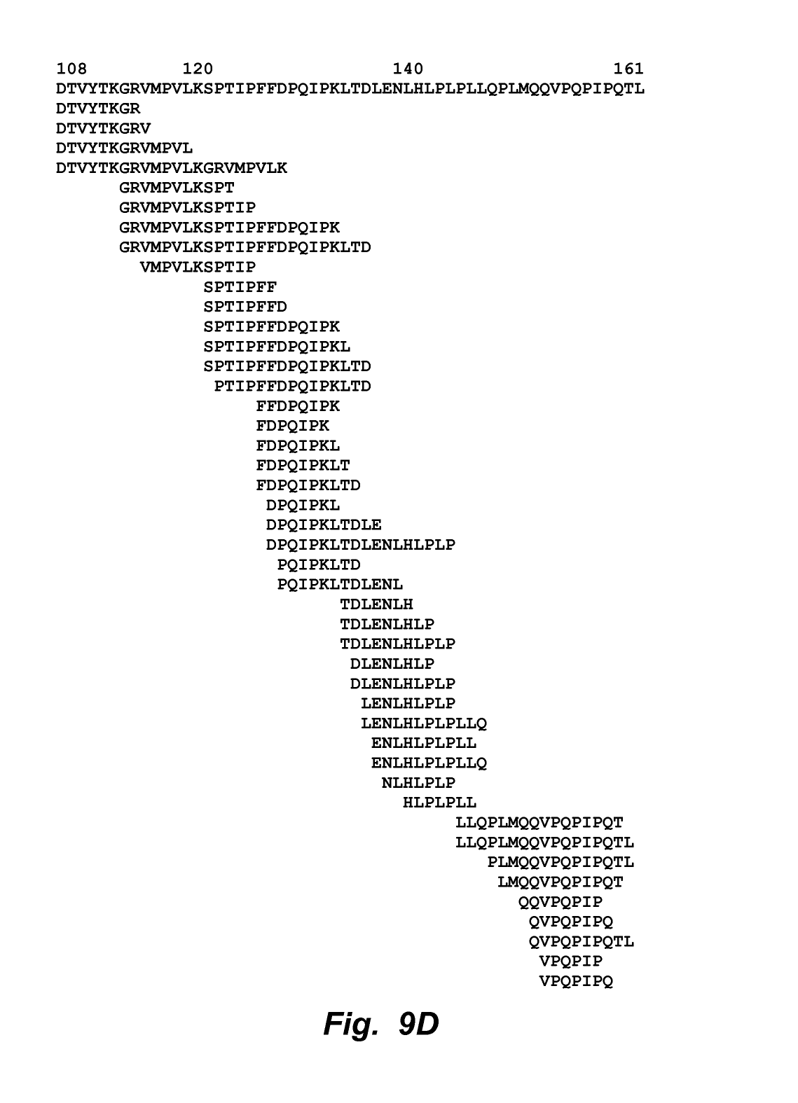

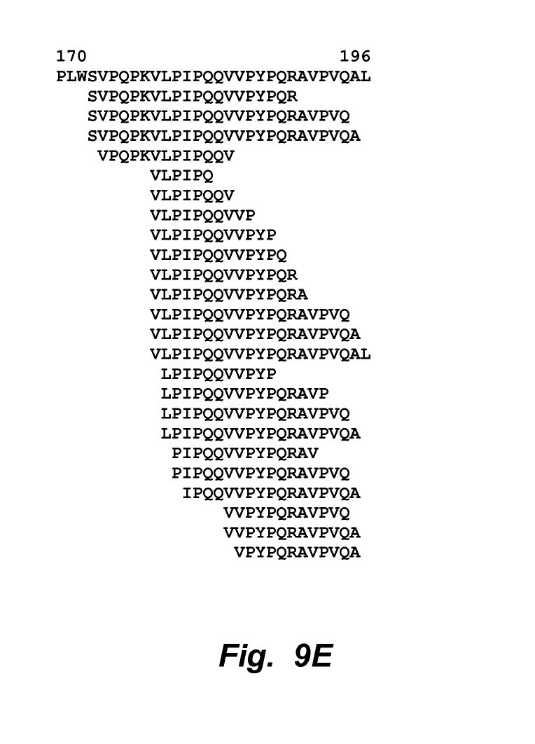

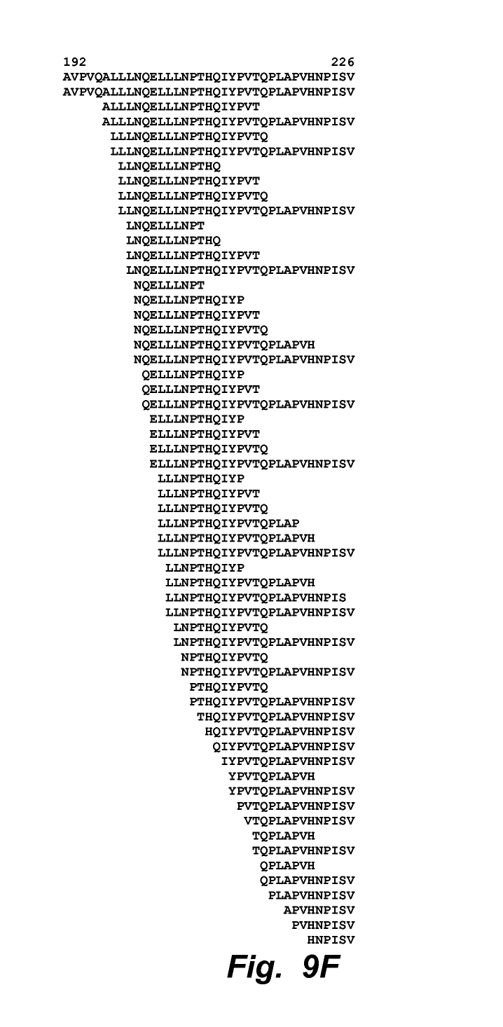

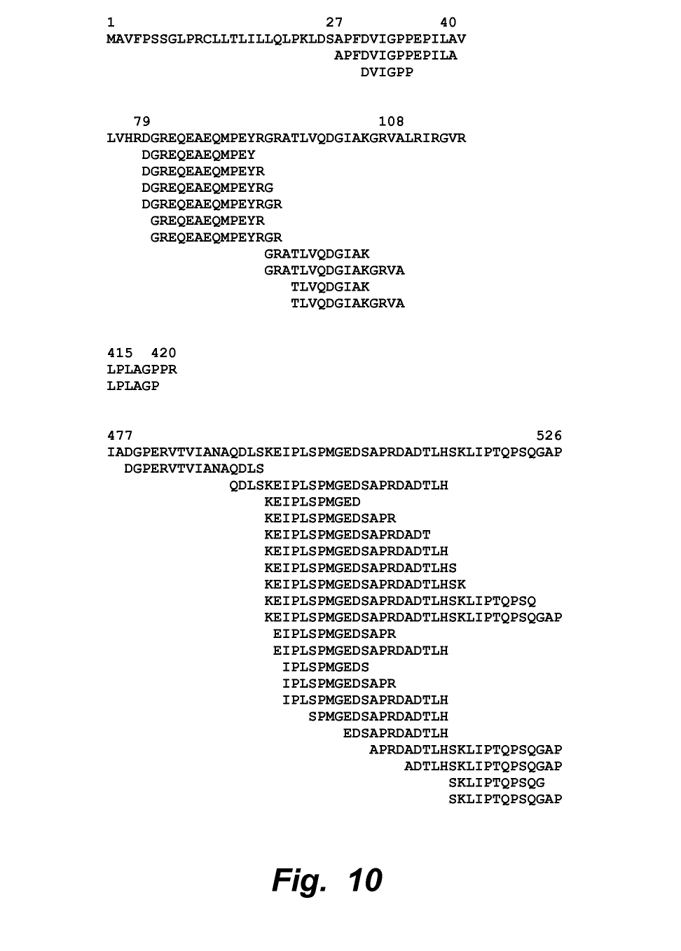

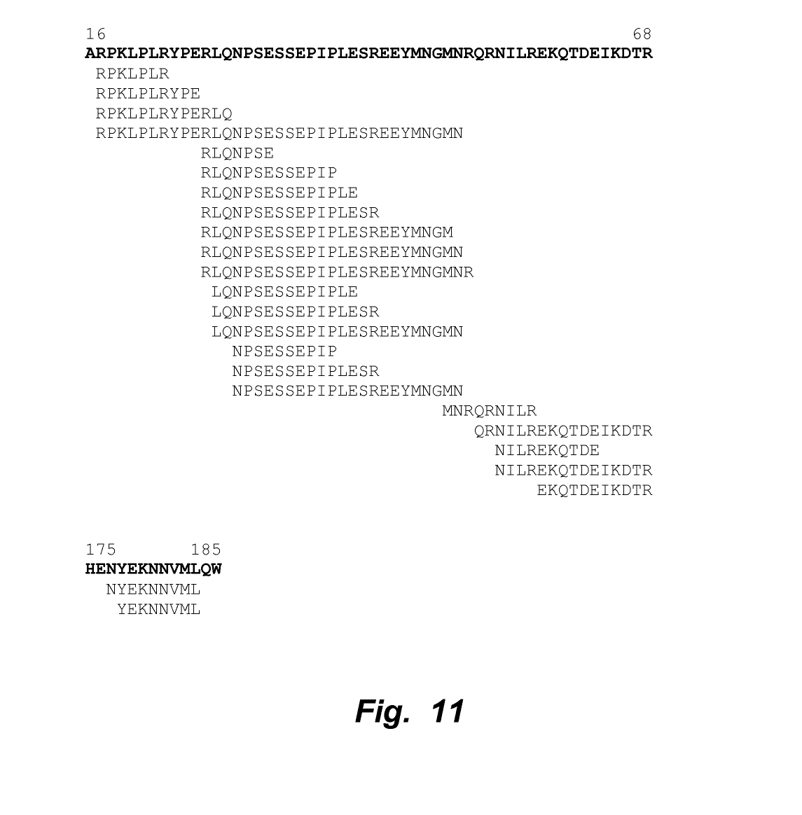

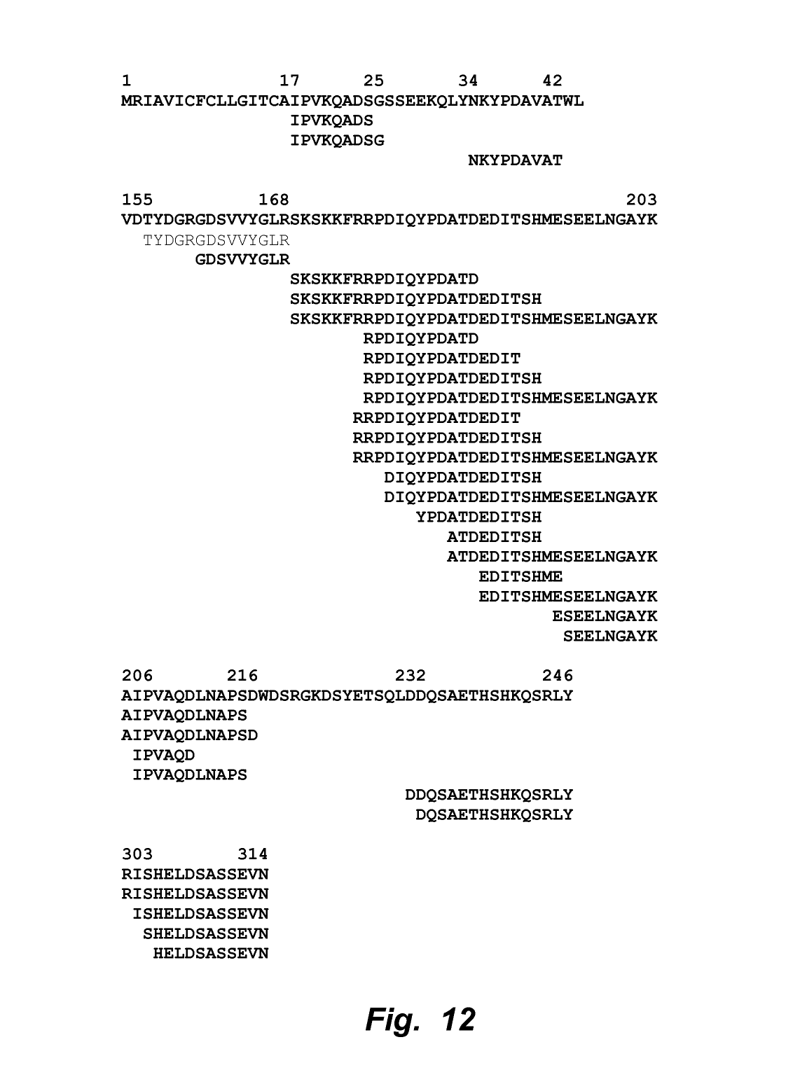

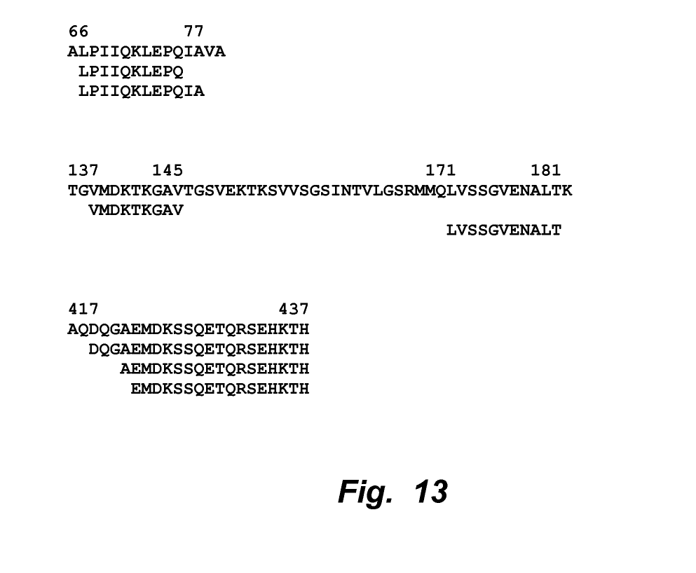

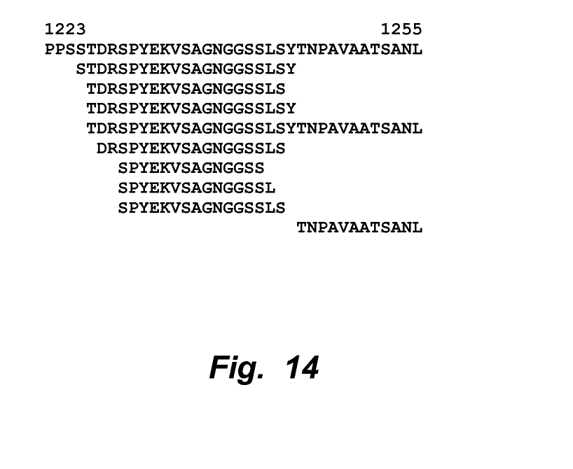

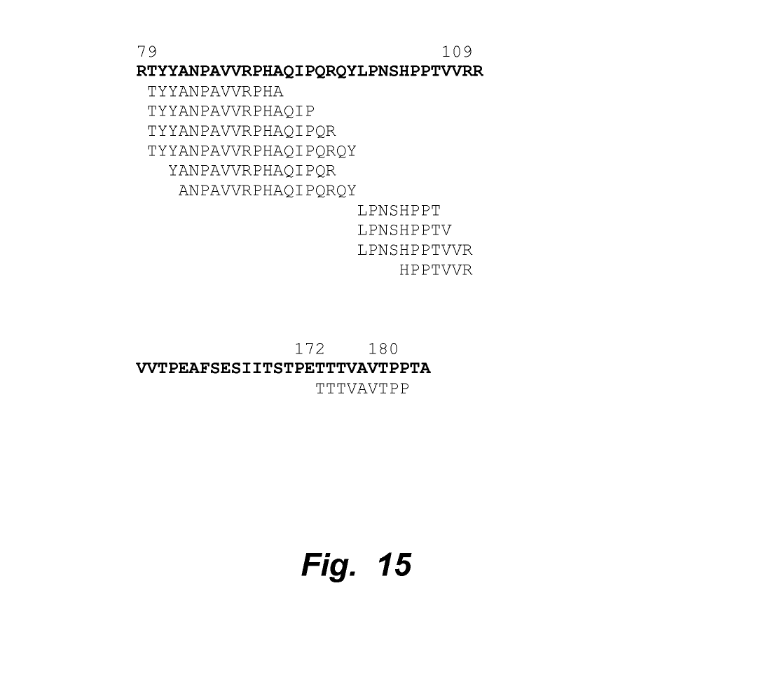

The majority (62%) of the identified peptides were derived from β-casein (see FIG. 7). Other major contributors to peptide fragments included polymeric immunoglobulin receptor, butyrophilin, αₛ₁-casein, osteopontin, κ-casein and mucin-1. Overall, 27 proteins with at least one unique peptide (meaning unique sequence, protein of origin and phosphorylation site) at p-value (MS-GFDB) or e-value (X!Tandem)≦0.01 were identified.

Identified peptides ranged from 6 to 37 amino acids in length. The average peptide length was 17.1 amino acids. Peptide masses ranged from 666 to 4269 Daltons, with an average of 1906.5 Daltons. This size distribution does not necessarily reflect biology, as larger peptides may be precipitated by TCA.

Thirty-two percent of peptides identified were phosphorylated at serine, threonine or tyrosine (163 unique peptides). The identified sites of phosphorylation were compared with known sites of phosphorylation from Uniprot. Phosphorylation sites that matched previous identifications in Uniprot are shown in Table 3 in italics. Not all phosphorylation sites could be determined with certainty—in many cases, tandem MS analysis could not differentiate between several sites of phosphorylation. In these instances, the possible phosphorylation sites were underlined. Phosphorylation sites that were determined were bolded. Thirty-five peptides (7%) had a previously unknown phosphorylation site. As some of these peptides had the same new phosphorylation site, the number of new phosphorylation sites was 18.

Bioactive Peptides.

Of the 537 peptides found, 72 shared at least 57% of their length with a known bioactive peptide from the compiled databases. One peptide in β-casein matches the literature exactly. The high sequence overlap between these identified peptides and those in the library suggests those matching may have similar bioactivity to the library peptide. Sixty-two (62) fragments are from β-casein and 10 are from 1c-casein (Table 4). All of these bioactive peptides matched were database entries from proteins known to exist in milk. Sixty-five (65) of these peptides matched antibacterial sequences.

TABLE 3
Antibacterial Peptides Identified in Skim Human Milk at 99% Confidence Level
TABLE 3

| SEQ | | | | | | |
|---|---|---|---|---|---|---|
| ID | | N^{o} | | | | MS- |
| NO. | Peptide sequence | phos | Uniprot Protein ID | Protein Name | X!Tandem | GFDB |
| 4 | PSPEADAPVLG [symbol] PEKEEAASEPPAAAPDA | 1 | sp\|O15234\|CASC3_HUMAN | cancer | X | |
| | | | | susceptibility | | |
| | | | | candidate gene 3 | | |
| | | | | protein | | |
| 9 | ITHRIHWESAS | 1 | sp\|P01024\|CO3_HUMAN | complement C3 | | X |
| 24 | AVADTRDQADGSRASVD | | sp\|P01833\|PIGR_HUMAN | polymeric | | X |
| | | | | immunoglobulin | | |
| | | | | receptor | | |
| 43 | DQADGSRASVDSGSSEEQGGSSRALVSTLVP | | sp\|P01833\|PIGR_HUMAN | polymeric | | X |
| | | | | immunoglobulin | | |
| | | | | receptor | | |
| 37 | DPRLFAEEKAVADTR | | sp\|P01833\|PIGR_HUMAN | polymeric | | X |
| | | | | immunoglobulin | | |
| | | | | receptor | | |
| 58 | LFAEEKAVADTRDQADGSR | | sp\|P01833\|PIGR_HUMAN | polymeric | | X |
| | | | | immunoglobulin | | |
| | | | | receptor | | |
| 13 | ADAAPDEKVLDSGFREIENK | | sp\|P01833\|PIGR_HUMAN | polymeric | | X |
| | | | | immunoglobulin | | |
| | | | | receptor | | |
| 22 | AVADTRDQADGS | | sp\|P01833\|PIGR_HUMAN | polymeric | | X |
| | | | | immunoglobulin | | |
| | | | | receptor | | |
| 18 | ASVDSGSSEEQGGSSRALVSTLVP | | sp\|P01833\|PIGR_HUMAN | polymeric | | X |
| | | | | immunoglobulin | | |
| | | | | receptor | | |
| 19 | ASVDSGSSEEQGGSSRALVSTLVPLG | | sp\|P01833\|PIGR_HUMAN | polymeric | | X |
| | | | | immunoglobulin | | |
| | | | | receptor | | |
| 12 | AAPDEKVLDSGFREIENK | | sp\|P01833\|PIGR_HUMAN | polymeric | | X |
| | | | | immunoglobulin | | |
| | | | | receptor | | |
| 60 | QADGSRASVDSGSSEEQGGSSRA | | sp\|P01833\|PIGR_HUMAN | polymeric | | X |
| | | | | immunoglobulin | | |
| | | | | receptor | | |
| 35 | DGSRASVDSGSSEEQGGSSR | | sp\|P01833\|PIGR_HUMAN | polymeric | | X |
| | | | | immunoglobulin | | |
| | | | | receptor | | |
| 71 | VDSGSSEEQGGSSRALVSTLVP | | sp\|P01833\|PIGR_HUMAN | polymeric | | X |
| | | | | immunoglobulin | | |
| | | | | receptor | | |
| 32 | AVADTRDQADGSRASVDSGSSEEQGGSSRALVSTLVP | | sp\|P01833\|PIGR_HUMAN | polymeric | | X |
| | | | | immunoglobulin | | |
| | | | | receptor | | |
| 23 | AVADTRDQADGSRAS | | sp\|P01833\|PIGR_HUMAN | polymeric | | X |
| | | | | immunoglobulin | | |
| | | | | receptor | | |
| 45 | DQADGSRASVDSGSSEEQGGSSRALVSTLVPLG | | sp\|P01833\|PIGR_HUMAN | polymeric | | X |
| | | | | immunoglobulin | | |
| | | | | receptor | | |
| 72 | VDSGSSEEQGGSSRALVSTLVPLG | | sp\|P01833\|PIGR_HUMAN | polymeric | | X |
| | | | | immunoglobulin | | |
| | | | | receptor | | |
| 17 | AIQDPRLFAEEKAVADTRDQADGS | | sp\|P01833\|PIGR_HUMAN | polymeric | | X |
| | | | | immunoglobulin | | |
| | | | | receptor | | |
| 49 | DSGSSEEQGGSSRALVSTLVP | | sp\|P01833\|PIGR_HUMAN | polymeric | | X |
| | | | | immunoglobulin | | |
| | | | | receptor | | |
| 53 | EEKAVADTRDQADGSR | | sp\|P01833\|PIGR_HUMAN | polymeric | | X |
| | | | | immunoglobulin | | |
| | | | | receptor | | |
| 21 | AVADTRDQADG | | sp\|P01833\|PIGR_HUMAN | polymeric | | X |
| | | | | immunoglobulin | | |
| | | | | receptor | | |
| 25 | AVADTRDQADGSRASVDSG | | sp\|P01833\|PIGR_HUMAN | polymeric | | X |
| | | | | immunoglobulin | | |
| | | | | receptor | | |
| 51 | DSGSSEEQGGSSRALVSTLVPLG | | sp\|P01833\|PIGR_HUMAN | polymeric | | X |
| | | | | immunoglobulin | | |
| | | | | receptor | | |
| 26 | AVADTRDQADGSRASVDSGSSEEQG | | sp\|P01833\|PIGR_HUMAN | polymeric | | X |
| | | | | immunoglobulin | | |
| | | | | receptor | | |
| 62 | SVDSGSSEEQGGSSRALVST | | sp\|P01833\|PIGR_HUMAN | polymeric | | X |
| | | | | immunoglobulin | | |
| | | | | receptor | | |
| 63 | SVDSGSSEEQGGSSRALVSTLVP | | sp\|P01833\|PIGR_HUMAN | polymeric | | X |
| | | | | immunoglobulin | | |
| | | | | receptor | | |
| 57 | KADAAPDEKVLDSGFREIENK | | sp\|P01833\|PIGR_HUMAN | polymeric | | X |
| | | | | immunoglobulin | | |
| | | | | receptor | | |
| 31 | AVADTRDQADGSRASVDSGSSEEQGGSSRALVST | | sp\|P01833\|PIGR_HUMAN | polymeric | | X |
| | | | | immunoglobulin | | |
| | | | | receptor | | |
| 27 | AVADTRDQADGSRASVDSGSSEEQGG | | sp\|P01833\|PIGR_HUMAN | polymeric | | X |
| | | | | immunoglobulin | | |
| | | | | receptor | | |
| 50 | DSGSSEEQGGSSRALVSTLVPL | | sp\|P01833\|PIGR_HUMAN | polymeric | | X |
| | | | | immunoglobulin | | |
| | | | | receptor | | |
| 14 | ADTRDQADGSRASVDSGSSEEQGGSSRA | | sp\|P01833\|PIGR_HUMAN | polymeric | X | X |
| | | | | immunoglobulin | | |
| | | | | receptor | | |
| 15 | AEEKAVADTRDQADGSR | | sp\|P01833\|PIGR_HUMAN | polymeric | X | X |
| | | | | immunoglobulin | | |
| | | | | receptor | | |
| 28 | AVADTRDQADGSRASVDSGSSEEQGGSS | | sp\|P01833\|PIGR_HUMAN | polymeric | X | X |
| | | | | immunoglobulin | | |
| | | | | receptor | | |
| 29 | AVADTRDQADGSRASVDSGSSEEQGGSSRA | | sp\|P01833\|PIGR_HUMAN | polymeric | X | X |
| | | | | immunoglobulin | | |
| | | | | receptor | | |
| 30 | AVADTRDQADGSRASVDSGSSEEQGGSSRAL | | sp\|P01833\|PIGR_HUMAN | polymeric | X | X |
| | | | | immunoglobulin | | |
| | | | | receptor | | |
| 34 | DAAPDEKVLDSGFREIENK | | sp\|P01833\|PIGR_HUMAN | polymeric | X | X |
| | | | | immunoglobulin | | |
| | | | | receptor | | |
| 36 | DGSRASVDSGSSEEQGGSSRA | | sp\|P01833\|PIGR_HUMAN | polymeric | X | X |
| | | | | immunoglobulin | | |
| | | | | receptor | | |
| 39 | DQADGSRASVDSGSSEEQGGSSRA | | sp\|P01833\|PIGR_HUMAN | polymeric | X | X |
| | | | | immunoglobulin | | |
| | | | | receptor | | |
| 40 | DQADGSRASVDSGSSEEQGGSSRAL | | sp\|P01833\|PIGR_HUMAN | polymeric | X | X |
| | | | | immunoglobulin | | |
| | | | | receptor | | |
| 42 | DQADGSRASVDSGSSEEQGGSSRALVST | | sp\|P01833\|PIGR_HUMAN | polymeric | X | X |
| | | | | immunoglobulin | | |
| | | | | receptor | | |
| 46 | DSGSSEEQGGSSRAL | | sp\|P01833\|PIGR_HUMAN | polymeric | X | X |
| | | | | immunoglobulin | | |
| | | | | receptor | | |
| 47 | DSGSSEEQGGSSRALV | | sp\|P01833\|PIGR_HUMAN | polymeric | X | X |
| | | | | immunoglobulin | | |
| | | | | receptor | | |
| 48 | DSGSSEEQGGSSRALVST | | sp\|P01833\|PIGR_HUMAN | polymeric | X | X |
| | | | | immunoglobulin | | |
| | | | | receptor | | |
| 55 | FAEEKAVADTRDQADGSR | | sp\|P01833\|PIGR_HUMAN | polymeric | X | X |
| | | | | immunoglobulin | | |
| | | | | receptor | | |
| 56 | FAEEKAVADTRDQADGSRASVDSGSSEEQGGSSRA | | sp\|P01833\|PIGR_HUMAN | polymeric | X | X |
| | | | | immunoglobulin | | |
| | | | | receptor | | |
| 59 | LFAEEKAVADTRDQADGSRASVDSGSSEEQGGSSRA | | sp\|P01833\|PIGR_HUMAN | polymeric | X | X |
| | | | | immunoglobulin | | |
| | | | | receptor | | |
| 61 | SVDSGSSEEQGGSSRA | | sp\|P01833\|PIGR_HUMAN | polymeric | X | X |
| | | | | immunoglobulin | | |
| | | | | receptor | | |
| 65 | SVDSGSSEEQGGSSRALVSTLVPLG | | sp\|P01833\|PIGR_HUMAN | polymeric | X | X |
| | | | | immunoglobulin | | |
| | | | | receptor | | |
| 68 | VADTRDQADGSRASVDSGSSEEQGGSS | | sp\|P01833\|PIGR_HUMAN | polymeric | X | X |
| | | | | immunoglobulin | | |
| | | | | receptor | | |
| 70 | VDSGSSEEQGGSSRA | | sp\|P01833\|PIGR_HUMAN | polymeric | X | X |
| | | | | immunoglobulin | | |
| | | | | receptor | | |
| 69 | VADTRDQADGSRASVDSGSSEEQGGSSRA | | sp\|P01833\|PIGR_HUMAN | polymeric | X | |
| | | | | immunoglobulin | | |
| | | | | receptor | | |
| 66 | TRDQADGSRASVDSGSSEEQGGSSRA | | sp\|P01833\|PIGR_HUMAN | polymeric | X | |
| | | | | immunoglobulin | | |
| | | | | receptor | | |
| 20 | AVADTRDQAD | | sp\|P01833\|PIGR_HUMAN | polymeric | X | |
| | | | | immunoglobulin | | |
| | | | | receptor | | |
| 52 | EEKAVADTRDQADG | | sp\|P01833\|PIGR_HUMAN | polymeric | X | |
| | | | | immunoglobulin | | |
| | | | | receptor | | |
| 54 | EKAVADTRDQADG | | sp\|P01833\|PIGR_HUMAN | polymeric | X | |
| | | | | immunoglobulin | | |
| | | | | receptor | | |
| 512 | DQADGSRASVDSGSSEEQGGSSR | | sp\|P01833\|PIGR_HUMAN | polymeric | | |
| | | | | immunoglobulin | | |
| | | | | receptor | | |
| 513 | GSSEEQGGSSRALV | | sp\|P01833\|PIGR_HUMAN | polymeric | | |
| | | | | immunoglobulin | | |
| | | | | receptor | | |
| 152 | LLLNQELLLNPTHQIYPVTQPLAPVHNPISV | | sp\|P05814\|CASB_HUMAN | beta-casein | | X |
| 110 | ETIESLS [symbol] EESITEYK | 2 | sp\|P05814\|CASB_HUMAN | beta-casein | | X |
| 110 | ETIESL [symbol] SEESITEYK | 2 | sp\|P05814\|CASB_HUMAN | beta-casein | | X |
| 111 | ETIESLSS [symbol] EESITEYKQ | 1 | sp\|P05814\|CASB_HUMAN | beta-casein | | X |
| 258 | [symbol] S [symbol] EESITEYK | 2 | sp\|P05814\|CASB_HUMAN | beta-casein | | X |
| 258 | [symbol] SSEESITEYK | 1 | sp\|P05814\|CASB_HUMAN | beta-casein | | X |
| 272 | TIESLS [symbol] EESITEYK | 2 | sp\|P05814\|CASB_HUMAN | beta-casein | | X |
| 248 | SLSSSEESITEYKQKVEK | | sp\|P05814\|CASB_HUMAN | beta-casein | | X |
| 256 | S [symbol] EESITEYK | 1 | sp\|P05814\|CASB_HUMAN | beta-casein | | X |
| 258 | S [symbol] EESITEYK | 2 | sp\|P05814\|CASB_HUMAN | beta-casein | | X |
| 256 | [symbol] SEESITEYK | 1 | sp\|P05814\|CASB_HUMAN | beta-casein | | X |
| 271 | TIESL [symbol] SSEESITEY | 1 | sp\|P05814\|CASB_HUMAN | beta-casein | | X |
| 113 | ETIESLS [symbol] EESITEYKQKVEK | 2 | sp\|P05814\|CASB_HUMAN | beta-casein | | X |
| 106 | ESLS [symbol] SEESITEYK | 1 | sp\|P05814\|CASB_HUMAN | beta-casein | | X |
| 154 | LLNPTHQIYPVTQPLAPVH | | sp\|P05814\|CASB_HUMAN | beta-casein | | X |
| 110 | ETIESLSSSEESITEYK | 1 | sp\|P05814\|CASB_HUMAN | beta-casein | | X |
| 269 | THQIYPVTQPLAPVHNPISV | | sp\|P05814\|CASB_HUMAN | beta-casein | | X |
| 106 | ESLS [symbol] EESITEYK | 2 | sp\|P05814\|CASB_HUMAN | beta-casein | | X |
| 111 | ETIESLS [symbol] SEESITEYKQ | 1 | sp\|P05814\|CASB_HUMAN | beta-casein | | X |
| 234 | RETIE [symbol] LSSSEESITEYK | 1 | sp\|P05814\|CASB_HUMAN | beta-casein | | X |
| 140 | KHEDQQQGEDEHQD | | sp\|P05814\|CASB_HUMAN | beta-casein | | X |
| 268 | TEYKQKVEKVKHED | | sp\|P05814\|CASB_HUMAN | beta-casein | | X |
| 189 | NQELLLNPTHQIYPVTQPLAPVH | | sp\|P05814\|CASB_HUMAN | beta-casein | | X |
| 315 | VVPYPQRAVPVQ | | sp\|P05814\|CASB_HUMAN | beta-casein | | X |
| 258 | S [symbol] SEESITEYK | 1 | sp\|P05814\|CASB_HUMAN | beta-casein | | X |
| 317 | YPVTQPLAPVH | | sp\|P05814\|CASB_HUMAN | beta-casein | | X |
| 258 | SS [symbol] EESITEYK | 1 | sp\|P05814\|CASB_HUMAN | beta-casein | | X |
| 236 | RETIE [symbol] LS [symbol] SEESITEYKQK | 2 | sp\|P05814\|CASB_HUMAN | beta-casein | | X |
| 179 | L [symbol] SSEESITEYKQKVEK | 1 | sp\|P05814\|CASB_HUMAN | beta-casein | | X |
| 109 | ETIESLSSSEESITEY | | sp\|P05814\|CASB_HUMAN | beta-casein | | X |
| 230 | RETIESLSS [symbol] EE | 1 | sp\|P05814\|CASB_HUMAN | beta-casein | | X |
| 314 | VVLPVPQPEIMEVPKAKDTVYTKGR | | sp\|P05814\|CASB_HUMAN | beta-casein | | X |
| 296 | VLPVPQPEIME | | sp\|P05814\|CASB_HUMAN | beta-casein | | X |
| 233 | RETIESLS [symbol] SEESITEY | 1 | sp\|P05814\|CASB_HUMAN | beta-casein | | X |
| 241 | RETIESLSS [symbol] EESITEYKQKVEKVKHE | 1 | sp\|P05814\|CASB_HUMAN | beta-casein | | X |
| 181 | NILPLAQPAVVLPVPQPEIMEVPK | | sp\|P05814\|CASB_HUMAN | beta-casein | | X |
| 313 | VVLPVPQPEIMEVPKAKDTVYTKG | | sp\|P05814\|CASB_HUMAN | beta-casein | | X |
| 201 | PQIPKLTDLENL | | sp\|P05814\|CASB_HUMAN | beta-casein | | X |
| 110 | ETIESL [symbol] SSEESITEYK | 1 | sp\|P05814\|CASB_HUMAN | beta-casein | | X |
| 273 | TIESLSS [symbol] EESITEYKQKVEK | 1 | sp\|P05814\|CASB_HUMAN | beta-casein | | X |
| 146 | LLLNPTHQIYPVT | | sp\|P05814\|CASB_HUMAN | beta-casein | | X |
| 148 | LLLNPTHQIYPVTQPLAP | | sp\|P05814\|CASB_HUMAN | beta-casein | | X |
| 159 | LLNQELLLNPTHQIYPVTQ | | sp\|P05814\|CASB_HUMAN | beta-casein | | X |
| 234 | RETIESL [symbol] S [symbol] EESITEYK | 2 | sp\|P05814\|CASB_HUMAN | beta-casein | | X |
| 272 | TIESL [symbol] SEESITEYK | 2 | sp\|P05814\|CASB_HUMAN | beta-casein | | X |
| 250 | SPTIPFFD | | sp\|P05814\|CASB_HUMAN | beta-casein | | X |
| 283 | VKHEDQQQGEDEHQDKIYPS | | sp\|P05814\|CASB_HUMAN | beta-casein | | X |
| 264 | TDLENLH | | sp\|P05814\|CASB_HUMAN | beta-casein | | X |
| 117 | FDPQIPK | | sp\|P05814\|CASB_HUMAN | beta-casein | | X |
| 200 | PQIPKLTD | | sp\|P05814\|CASB_HUMAN | beta-casein | | X |
| 311 | VVLPVPQPEIMEVPKAKDTVYT | | sp\|P05814\|CASB_HUMAN | beta-casein | | X |
| 236 | RETIESL [symbol] S [symbol] EESITEYKQK | 2 | sp\|P05814\|CASB_HUMAN | beta-casein | | X |
| 247 | [symbol] LS [symbol] SEESITEYK | 2 | sp\|P05814\|CASB_HUMAN | beta-casein | | X |
| 245 | [symbol] EESITEYKQKVE | 1 | sp\|P05814\|CASB_HUMAN | beta-casein | | X |
| 113 | ETIESLS [symbol] SEESITEYKQKVEK | 1 | sp\|P05814\|CASB_HUMAN | beta-casein | | X |
| 199 | PLMQQVPQPIPQTL | | sp\|P05814\|CASB_HUMAN | beta-casein | | X |
| 233 | RETIESL [symbol] SEESITEY | 2 | sp\|P05814\|CASB_HUMAN | beta-casein | | X |
| 236 | RETIESLS [symbol] SEESITEYKQK | 1 | sp\|P05814\|CASB_HUMAN | beta-casein | | X |
| 85 | DLENLHLP | | sp\|P05814\|CASB_HUMAN | beta-casein | | X |
| 109 | ETIESLSS [symbol] EESITEY | 1 | sp\|P05814\|CASB_HUMAN | beta-casein | | X |
| 157 | LLNQELLLNPTHQ | | sp\|P05814\|CASB_HUMAN | beta-casein | | X |
| 233 | RETIESL [symbol] S [symbol] EESITEY | 2 | sp\|P05814\|CASB_HUMAN | beta-casein | | X |
| 114 | EVPKAKDT | | sp\|P05814\|CASB_HUMAN | beta-casein | | X |
| 288 | VLPIPQQVVPYPQ | | sp\|P05814\|CASB_HUMAN | beta-casein | | X |
| 83 | DEHQDKI | | sp\|P05814\|CASB_HUMAN | beta-casein | | X |
| 271 | TIESLSSSEESITEY | | sp\|P05814\|CASB_HUMAN | beta-casein | | X |
| 179 | LSSSEESITEYKQKVEK | | sp\|P05814\|CASB_HUMAN | beta-casein | | X |
| 121 | FFDPQIPK | | sp\|P05814\|CASB_HUMAN | beta-casein | | X |
| 118 | FDPQIPKL | | sp\|P05814\|CASB_HUMAN | beta-casein | | X |
| 300 | VPKAKDTVYTKG | | sp\|P05814\|CASB_HUMAN | beta-casein | | X |
| 234 | RE [symbol] IESLSS [symbol] EESITEYK | 2 | sp\|P05814\|CASB_HUMAN | beta-casein | | X |
| 82 | AVPVQALLLNQELLLNPTHQIYPVTQPLAPVHNPISV | | sp\|P05814\|CASB_HUMAN | beta-casein | | X |
| 310 | VVLPVPQPEIMEVPKAKDT | | sp\|P05814\|CASB_HUMAN | beta-casein | | X |
| 238 | RETIESL [symbol] SSEESITEYKQKVEK | 1 | sp\|P05814\|CASB_HUMAN | beta-casein | | X |
| 236 | RETIESLS [symbol] EESITEYKQK | 2 | sp\|P05814\|CASB_HUMAN | beta-casein | | X |
| 286 | VLPIPQQVVP | | sp\|P05814\|CASB_HUMAN | beta-casein | | X |
| 86 | DLENLHLPLP | | sp\|P05814\|CASB_HUMAN | beta-casein | | X |
| 294 | VLPVPQPEI | | sp\|P05814\|CASB_HUMAN | beta-casein | | X |
| 234 | RE [symbol] IESLSSSEESITEYK | 1 | sp\|P05814\|CASB_HUMAN | beta-casein | | X |
| 135 | IESLS [symbol] EESITEYK | 2 | sp\|P05814\|CASB_HUMAN | beta-casein | | X |
| 234 | RETIESLS [symbol] SEESITEYK | 2 | sp\|P05814\|CASB_HUMAN | beta-casein | | X |
| 247 | SL [symbol] S [symbol] EESITEYK | 2 | sp\|P05814\|CASB_HUMAN | beta-casein | | X |
| 233 | RETIESLSS [symbol] EESITEY | 1 | sp\|P05814\|CASB_HUMAN | beta-casein | | X |
| 99 | ELLLNPTHQIYPVT | | sp\|P05814\|CASB_HUMAN | beta-casein | | X |
| 234 | RETIESL [symbol] SEESITEYK | 2 | sp\|P05814\|CASB_HUMAN | beta-casein | | X |
| 80 | AQPAVVLPVPQPEIMEVPKAKDTVYTK | | sp\|P05814\|CASB_HUMAN | beta-casein | | X |
| 180 | MEVPKAKDTVYTKGR | | sp\|P05814\|CASB_HUMAN | beta-casein | | X |
| 233 | RETIESLS [symbol] EESITEY | 2 | sp\|P05814\|CASB_HUMAN | beta-casein | | X |
| 116 | EVPKAKDTVYTK | | sp\|P05814\|CASB_HUMAN | beta-casein | | X |
| 123 | GEDEHQDKIYPS | | sp\|P05814\|CASB_HUMAN | beta-casein | | X |
| 229 | RETIESLS [symbol] | 1 | sp\|P05814\|CASB_HUMAN | beta-casein | | X |
| 122 | GEDEHQDK | | sp\|P05814\|CASB_HUMAN | beta-casein | | X |
| 234 | RETIE [symbol] L [symbol] SSEESITEYK | 2 | sp\|P05814\|CASB_HUMAN | beta-casein | | X |
| 221 | QPAVVLPVPQPEIMEVPKAKDTVYT | | sp\|P05814\|CASB_HUMAN | beta-casein | | X |
| 242 | RETIESLSS [symbol] EESITEYKQKVEKVKHEDQQQG | 1 | sp\|P05814\|CASB_HUMAN | beta-casein | | X |
| 138 | KDTVYTKGRVMPVL | | sp\|P05814\|CASB_HUMAN | beta-casein | | X |
| 179 | L [symbol] SEESITEYKQKVEK | 2 | sp\|P05814\|CASB_HUMAN | beta-casein | | X |
| 231 | RETIESLSS [symbol] EESI | 1 | sp\|P05814\|CASB_HUMAN | beta-casein | | X |
| 259 | [symbol] SSEESITEYKQKVE | 1 | sp\|P05814\|CASB_HUMAN | beta-casein | | X |
| 277 | VEKVKHEDQQQGEDEHQDKIYPS | | sp\|P05814\|CASB_HUMAN | beta-casein | | X |
| 234 | RETIE [symbol] LS [symbol] SEESITEYK | 2 | sp\|P05814\|CASB_HUMAN | beta-casein | | X |
| 97 | EKVKHEDQQQGEDEHQDK | | sp\|P05814\|CASB_HUMAN | beta-casein | | X |
| 177 | LPVPQPEIMEVPK | | sp\|P05814\|CASB_HUMAN | beta-casein | | X |
| 105 | ESLSSSEESITE | | sp\|P05814\|CASB_HUMAN | beta-casein | | X |
| 272 | [symbol] IESLSSSEESITEYK | 1 | sp\|P05814\|CASB_HUMAN | beta-casein | | X |
| 236 | RETIESL [symbol] SSEESITEYKQK | 1 | sp\|P05814\|CASB_HUMAN | beta-casein | | X |
| 95 | EIMEVPK | | sp\|P05814\|CASB_HUMAN | beta-casein | | X |
| 81 | AQPAVVLPVPQPEIMEVPKAKDTVYTKG | | sp\|P05814\|CASB_HUMAN | beta-casein | | X |
| 109 | ETIESLS [symbol] SEESITEY | 1 | sp\|P05814\|CASB_HUMAN | beta-casein | | X |
| 195 | PIPQQVVPYPQRAV | | sp\|P05814\|CASB_HUMAN | beta-casein | | X |
| 263 | SVPQPKVLPIPQQVVPYPQRAVPVQA | | sp\|P05814\|CASB_HUMAN | beta-casein | | X |
| 115 | EVPKAKDTVYT | | sp\|P05814\|CASB_HUMAN | beta-casein | | X |
| 259 | S [symbol] SEESITEYKQKVE | 1 | sp\|P05814\|CASB_HUMAN | beta-casein | | X |
| 102 | ENLHLPLPLL | | sp\|P05814\|CASB_HUMAN | beta-casein | | X |
| 301 | VPQPIP | | sp\|P05814\|CASB_HUMAN | beta-casein | | X |
| 254 | [symbol] SEESITE | 1 | sp\|P05814\|CASB_HUMAN | beta-casein | | X |
| 209 | QELLLNPTHQIYPVT | | sp\|P05814\|CASB_HUMAN | beta-casein | | X |
| 143 | LENLHLPLP | | sp\|P05814\|CASB_HUMAN | beta-casein | | X |
| 178 | L [symbol] SEESITEYK | 2 | sp\|P05814\|CASB_HUMAN | beta-casein | | X |
| 113 | ETIESL [symbol] SEESITEYKQKVEK | 2 | sp\|P05814\|CASB_HUMAN | beta-casein | | X |
| 88 | DPQIPKLTDLE | | sp\|P05814\|CASB_HUMAN | beta-casein | | X |
| 74 | AKDTVYTKGRVMPVLK | | sp\|P05814\|CASB_HUMAN | beta-casein | X | X |
| 76 | ALLLNQELLLNPTHQIYPVTQPLAPVHNPISV | | sp\|P05814\|CASB_HUMAN | beta-casein | X | X |
| 77 | APVHNPISV | | sp\|P05814\|CASB_HUMAN | beta-casein | X | X |
| 78 | AQPAVVLPVPQPEIMEVPK | | sp\|P05814\|CASB_HUMAN | beta-casein | X | X |
| 79 | AQPAVVLPVPQPEIMEVPKAKDTVYT | | sp\|P05814\|CASB_HUMAN | beta-casein | X | X |
| 84 | DEHQDKIYP | | sp\|P05814\|CASB_HUMAN | beta-casein | X | X |
| 90 | DTVYTKGR | | sp\|P05814\|CASB_HUMAN | beta-casein | X | X |
| 91 | DTVYTKGRV | | sp\|P05814\|CASB_HUMAN | beta-casein | X | X |
| 92 | DTVYTKGRVMPVL | | sp\|P05814\|CASB_HUMAN | beta-casein | X | X |
| 93 | DTVYTKGRVMPVLK | | sp\|P05814\|CASB_HUMAN | beta-casein | X | X |
| 94 | EESITEYK | | sp\|P05814\|CASB_HUMAN | beta-casein | X | X |
| 98 | ELLLNPTHQIYP | | sp\|P05814\|CASB_HUMAN | beta-casein | X | X |
| 100 | ELLLNPTHQIYPVTQ | | sp\|P05814\|CASB_HUMAN | beta-casein | X | X |
| 101 | ELLLNPTHQIYPVTQPLAPVHNPISV | | sp\|P05814\|CASB_HUMAN | beta-casein | X | X |
| 104 | ESITEYK | | sp\|P05814\|CASB_HUMAN | beta-casein | X | X |
| 106 | ESLSSSEESITEYK | | sp\|P05814\|CASB_HUMAN | beta-casein | X | X |
| 108 | ETIESLSSSEESITE | | sp\|P05814\|CASB_HUMAN | beta-casein | X | X |
| 110 | ETIESLS [symbol] SEESITEYK | 1 | sp\|P05814\|CASB_HUMAN | beta-casein | X | X |
| 110 | ETIESLSS [symbol] EESITEYK | 1 | sp\|P05814\|CASB_HUMAN | beta-casein | X | X |
| 110 | ETIESLSSSEESITEYK | | sp\|P05814\|CASB_HUMAN | beta-casein | X | X |
| 113 | ETIESLSSSEESITEYKQKVEK | | sp\|P05814\|CASB_HUMAN | beta-casein | X | X |
| 127 | GRVMPVLKSPTIPFFDPQIPK | | sp\|P05814\|CASB_HUMAN | beta-casein | X | X |
| 128 | GRVMPVLKSPTIPFFDPQIPKLTD | | sp\|P05814\|CASB_HUMAN | beta-casein | X | X |
| 130 | HEDQQQGEDEHQDKIYP | | sp\|P05814\|CASB_HUMAN | beta-casein | X | X |
| 133 | HNPISV | | sp\|P05814\|CASB_HUMAN | beta-casein | X | X |
| 134 | HQIYPVTQPLAPVHNPISV | | sp\|P05814\|CASB_HUMAN | beta-casein | X | X |
| 135 | IESLS [symbol] SEESITEYK | 1 | sp\|P05814\|CASB_HUMAN | beta-casein | X | X |
| 135 | IESLSSSEESITEYK | | sp\|P05814\|CASB_HUMAN | beta-casein | X | X |
| 136 | IPQQVVPYPQRAVPVQA | | sp\|P05814\|CASB_HUMAN | beta-casein | X | X |
| 139 | KDTVYTKGRVMPVLK | | sp\|P05814\|CASB_HUMAN | beta-casein | X | X |
| 142 | KVEKVKHEDQQQGEDEHQDK | | sp\|P05814\|CASB_HUMAN | beta-casein | X | X |
| 144 | LENLHLPLPLLQ | | sp\|P05814\|CASB_HUMAN | beta-casein | X | X |
| 149 | LLLNPTHQIYPVTQPLAPVH | | sp\|P05814\|CASB_HUMAN | beta-casein | X | X |
| 150 | LLLNPTHQIYPVTQPLAPVHNPISV | | sp\|P05814\|CASB_HUMAN | beta-casein | X | X |
| 155 | LLNPTHQIYPVTQPLAPVHNPIS | | sp\|P05814\|CASB_HUMAN | beta-casein | X | X |
| 156 | LLNPTHQIYPVTQPLAPVHNPISV | | sp\|P05814\|CASB_HUMAN | beta-casein | X | X |
| 158 | LLNQELLLNPTHQIYPVT | | sp\|P05814\|CASB_HUMAN | beta-casein | X | X |
| 160 | LLNQELLLNPTHQIYPVTQPLAPVHNPISV | | sp\|P05814\|CASB_HUMAN | beta-casein | X | X |
| 161 | LLQPLMQQVPQPIPQT | | sp\|P05814\|CASB_HUMAN | beta-casein | X | X |
| 162 | LLQPLMQQVPQPIPQTL | | sp\|P05814\|CASB_HUMAN | beta-casein | X | X |
| 164 | LNPTHQIYPVTQ | | sp\|P05814\|CASB_HUMAN | beta-casein | X | X |
| 166 | LNQELLLNPT | | sp\|P05814\|CASB_HUMAN | beta-casein | X | X |
| 167 | LNQELLLNPTHQ | | sp\|P05814\|CASB_HUMAN | beta-casein | X | X |
| 169 | LNQELLLNPTHQIYPVTQPLAPVHNPISV | | sp\|P05814\|CASB_HUMAN | beta-casein | X | X |
| 170 | LPIPQQVVPYP | | sp\|P05814\|CASB_HUMAN | beta-casein | X | X |
| 171 | LPIPQQVVPYPQRAVP | | sp\|P05814\|CASB_HUMAN | beta-casein | X | X |
| 172 | LPIPQQVVPYPQRAVPVQ | | sp\|P05814\|CASB_HUMAN | beta-casein | X | X |
| 173 | LPIPQQVVPYPQRAVPVQA | | sp\|P05814\|CASB_HUMAN | beta-casein | X | X |
| 178 | LS [symbol] SEESITEYK | 1 | sp\|P05814\|CASB_HUMAN | beta-casein | X | X |
| 178 | LSS [symbol] EESITEYK | 1 | sp\|P05814\|CASB_HUMAN | beta-casein | X | X |
| 178 | LS [symbol] EESITEYK | 2 | sp\|P05814\|CASB_HUMAN | beta-casein | X | X |
| 178 | LSSSEESITEYK | | sp\|P05814\|CASB_HUMAN | beta-casein | X | X |
| 183 | NPTHQIYPVTQ | | sp\|P05814\|CASB_HUMAN | beta-casein | X | X |
| 184 | NPTHQIYPVTQPLAPVHNPISV | | sp\|P05814\|CASB_HUMAN | beta-casein | X | X |
| 187 | NQELLLNPTHQIYPVT | | sp\|P05814\|CASB_HUMAN | beta-casein | X | X |
| 188 | NQELLLNPTHQIYPVTQ | | sp\|P05814\|CASB_HUMAN | beta-casein | X | X |
| 190 | NQELLLNPTHQIYPVTQPLAPVHNPISV | | sp\|P05814\|CASB_HUMAN | beta-casein | X | X |
| 191 | PAVVLPVPQPEI | | sp\|P05814\|CASB_HUMAN | beta-casein | X | X |
| 192 | PAVVLPVPQPEIME | | sp\|P05814\|CASB_HUMAN | beta-casein | X | X |
| 196 | PIPQQVVPYPQRAVPVQ | | sp\|P05814\|CASB_HUMAN | beta-casein | X | X |
| 197 | PLAPVHNPISV | | sp\|P05814\|CASB_HUMAN | beta-casein | X | X |
| 198 | PLAQPAVVLPVPQPEI | | sp\|P05814\|CASB_HUMAN | beta-casein | X | X |
| 202 | PTHQIYPVTQ | | sp\|P05814\|CASB_HUMAN | beta-casein | X | X |
| 203 | PTHQIYPVTQPLAPVHNPISV | | sp\|P05814\|CASB_HUMAN | beta-casein | X | X |
| 204 | PTIPFFDPQIPKLTD | | sp\|P05814\|CASB_HUMAN | beta-casein | X | X |
| 207 | PVTQPLAPVHNPISV | | sp\|P05814\|CASB_HUMAN | beta-casein | X | X |
| 208 | QELLLNPTHQIYP | | sp\|P05814\|CASB_HUMAN | beta-casein | X | X |
| 210 | QELLLNPTHQIYPVTQPLAPVHNPISV | | sp\|P05814\|CASB_HUMAN | beta-casein | X | X |
| 212 | QKVEKVK | | sp\|P05814\|CASB_HUMAN | beta-casein | X | X |
| 215 | QKVEKVKHEDQQQGEDEHQDK | | sp\|P05814\|CASB_HUMAN | beta-casein | X | X |
| 216 | QPAVVLPVPQPEI | | sp\|P05814\|CASB_HUMAN | beta-casein | X | X |
| 217 | QPAVVLPVPQPEIM | | sp\|P05814\|CASB_HUMAN | beta-casein | X | X |
| 218 | QPAVVLPVPQPEIMEVPK | | sp\|P05814\|CASB_HUMAN | beta-casein | X | X |
| 219 | QPAVVLPVPQPEIMEVPKA | | sp\|P05814\|CASB_HUMAN | beta-casein | X | X |
| 220 | QPAVVLPVPQPEIMEVPKAK | | sp\|P05814\|CASB_HUMAN | beta-casein | X | X |
| 222 | QPAVVLPVPQPEIMEVPKAKDTVYTK | | sp\|P05814\|CASB_HUMAN | beta-casein | X | X |
| 224 | QPLAPVHNPISV | | sp\|P05814\|CASB_HUMAN | beta-casein | X | X |
| 227 | QVPQPIPQTL | | sp\|P05814\|CASB_HUMAN | beta-casein | X | X |
| 230 | RETIESLSSSEE | | sp\|P05814\|CASB_HUMAN | beta-casein | X | X |
| 232 | RETIESL [symbol] SSEESITE | 1 | sp\|P05814\|CASB_HUMAN | beta-casein | X | X |
| 233 | RETIESLSSSEESITEY | | sp\|P05814\|CASB_HUMAN | beta-casein | X | X |
| 234 | RETIESLSS [symbol] EESITEYK | 1 | sp\|P05814\|CASB_HUMAN | beta-casein | X | X |
| 234 | RETIESLS [symbol] SEESITEYK | 1 | sp\|P05814\|CASB_HUMAN | beta-casein | X | X |
| 234 | RETIESL [symbol] SSEESITEYK | 1 | sp\|P05814\|CASB_HUMAN | beta-casein | X | X |
| 234 | RETIESLS [symbol] EESITEYK | 2 | sp\|P05814\|CASB_HUMAN | beta-casein | X | X |
| 234 | RETIESLSSSEESITEYK | | sp\|P05814\|CASB_HUMAN | beta-casein | X | X |
| 236 | RETIESLSSSEESITEYKQK | | sp\|P05814\|CASB_HUMAN | beta-casein | X | X |
| 237 | RETIESLSSSEESITEYKQKVE | | sp\|P05814\|CASB_HUMAN | beta-casein | X | X |
| 238 | RETIESLSS [symbol] EESITEYKQKVEK | 1 | sp\|P05814\|CASB_HUMAN | beta-casein | X | X |
| 238 | RETIESLS [symbol] SEESITEYKQKVEK | 1 | sp\|P05814\|CASB_HUMAN | beta-casein | X | X |
| 244 | [symbol] SEESITEYK | 1 | sp\|P05814\|CASB_HUMAN | beta-casein | X | X |
| 244 | SEESITEYK | | sp\|P05814\|CASB_HUMAN | beta-casein | X | X |
| 247 | SLSS [symbol] EESITEYK | 1 | sp\|P05814\|CASB_HUMAN | beta-casein | X | X |
| 247 | SLS [symbol] SEESITEYK | 1 | sp\|P05814\|CASB_HUMAN | beta-casein | X | X |
| 247 | SLS [symbol] EESITEYK | 2 | sp\|P05814\|CASB_HUMAN | beta-casein | X | X |
| 247 | SLSSSEESITEYK | | sp\|P05814\|CASB_HUMAN | beta-casein | X | X |
| 251 | SPTIPFFDPQIPK | | sp\|P05814\|CASB_HUMAN | beta-casein | X | X |
| 252 | SPTIPFFDPQIPKL | | sp\|P05814\|CASB_HUMAN | beta-casein | X | X |
| 253 | SPTIPFFDPQIPKLTD | | sp\|P05814\|CASB_HUMAN | beta-casein | X | X |
| 256 | [symbol] EESITEYK | 2 | sp\|P05814\|CASB_HUMAN | beta-casein | X | X |
| 256 | SSEESITEYK | | sp\|P05814\|CASB_HUMAN | beta-casein | X | X |
| 258 | [symbol] SEESITEYK | 2 | sp\|P05814\|CASB_HUMAN | beta-casein | X | X |
| 258 | SSSEESITEYK | | sp\|P05814\|CASB_HUMAN | beta-casein | X | X |
| 262 | SVPQPKVLPIPQQVVPYPQRAVPVQ | | sp\|P05814\|CASB_HUMAN | beta-casein | X | X |
| 266 | TDLENLHLPLP | | sp\|P05814\|CASB_HUMAN | beta-casein | X | X |
| 267 | TEYKQKVE | | sp\|P05814\|CASB_HUMAN | beta-casein | X | X |
| 270 | TIESLSSSEESITE | | sp\|P05814\|CASB_HUMAN | beta-casein | X | X |
| 272 | TIESLS [symbol] SEESITEYK | 1 | sp\|P05814\|CASB_HUMAN | beta-casein | X | X |
| 272 | TIESLSS [symbol] EESITEYK | 1 | sp\|P05814\|CASB_HUMAN | beta-casein | X | X |
| 272 | TIESLSSSEESITEYK | | sp\|P05814\|CASB_HUMAN | beta-casein | X | X |
| 273 | TIESLSSSEESITEYKQKVEK | | sp\|P05814\|CASB_HUMAN | beta-casein | X | X |
| 274 | TQPLAPVH | | sp\|P05814\|CASB_HUMAN | beta-casein | X | X |
| 275 | TQPLAPVHNPISV | | sp\|P05814\|CASB_HUMAN | beta-casein | X | X |
| 276 | VEKVKHEDQQQGEDEHQDK | | sp\|P05814\|CASB_HUMAN | beta-casein | X | X |
| 278 | VEPIPYGFLPQ | | sp\|P05814\|CASB_HUMAN | beta-casein | X | X |
| 279 | VKHEDQQQGEDEHQ | | sp\|P05814\|CASB_HUMAN | beta-casein | X | X |
| 280 | VKHEDQQQGEDEHQD | | sp\|P05814\|CASB_HUMAN | beta-casein | X | X |
| 281 | VKHEDQQQGEDEHQDK | | sp\|P05814\|CASB_HUMAN | beta-casein | X | X |
| 285 | VLPIPQQV | | sp\|P05814\|CASB_HUMAN | beta-casein | X | X |
| 287 | VLPIPQQVVPYP | | sp\|P05814\|CASB_HUMAN | beta-casein | X | X |
| 289 | VLPIPQQVVPYPQR | | sp\|P05814\|CASB_HUMAN | beta-casein | X | X |
| 290 | VLPIPQQVVPYPQRA | | sp\|P05814\|CASB_HUMAN | beta-casein | X | X |
| 291 | VLPIPQQVVPYPQRAVPVQ | | sp\|P05814\|CASB_HUMAN | beta-casein | X | X |
| 292 | VLPIPQQVVPYPQRAVPVQA | | sp\|P05814\|CASB_HUMAN | beta-casein | X | X |
| 295 | VLPVPQPEIM | | sp\|P05814\|CASB_HUMAN | beta-casein | X | X |
| 297 | VLPVPQPEIMEVPK | | sp\|P05814\|CASB_HUMAN | beta-casein | X | X |
| 299 | VPKAKDTVYT | | sp\|P05814\|CASB_HUMAN | beta-casein | X | X |
| 304 | VPYPQRAVPVQA | | sp\|P05814\|CASB_HUMAN | beta-casein | X | X |
| 305 | VTQPLAPVHNPISV | | sp\|P05814\|CASB_HUMAN | beta-casein | X | X |
| 306 | VVLPVPQPEIME | | sp\|P05814\|CASB_HUMAN | beta-casein | X | X |
| 307 | VVLPVPQPEIMEVPK | | sp\|P05814\|CASB_HUMAN | beta-casein | X | X |
| 308 | VVLPVPQPEIMEVPKA | | sp\|P05814\|CASB_HUMAN | beta-casein | X | X |
| 312 | VVLPVPQPEIMEVPKAKDTVYTK | | sp\|P05814\|CASB_HUMAN | beta-casein | X | X |
| 316 | VVPYPQRAVPVQA | | sp\|P05814\|CASB_HUMAN | beta-casein | X | X |
| 318 | YPVTQPLAPVHNPISV | | sp\|P05814\|CASB_HUMAN | beta-casein | X | X |
| 272 | TIESL [symbol] SSEESITEYK | 1 | sp\|P05814\|CASB_HUMAN | beta-casein | X | |
| 110 | ETIESLSSSEESITEYK | 2 | sp\|P05814\|CASB_HUMAN | beta-casein | X | |
| 165 | LNPTHQIYPVTQPLAPVHNPISV | | sp\|P05814\|CASB_HUMAN | beta-casein | X | |
| 110 | ETIESLSSSEESITEYK | 1 | sp\|P05814\|CASB_HUMAN | beta-casein | X | |
| 236 | RETIESLSSSEESITEYKQK | 1 | sp\|P05814\|CASB_HUMAN | beta-casein | X | |
| 211 | QIYPVTQPLAPVHNPISV | | sp\|P05814\|CASB_HUMAN | beta-casein | X | |
| 239 | RETIESLS [symbol] SEESITEYKQKVEKV | 1 | sp\|P05814\|CASB_HUMAN | beta-casein | X | |
| 239 | RETIESL [symbol] SSEESITEYKQKVEKV | 1 | sp\|P05814\|CASB_HUMAN | beta-casein | X | |
| 261 | SVPQPKVLPIPQQVVPYPQR | | sp\|P05814\|CASB_HUMAN | beta-casein | X | |
| 110 | ETIE [symbol] LSSSEESITEYK | 2 | sp\|P05814\|CASB_HUMAN | beta-casein | X | |
| 247 | SL [symbol] SSEESITEYK | 2 | sp\|P05814\|CASB_HUMAN | beta-casein | X | |
| 186 | NQELLLNPTHQIYP | | sp\|P05814\|CASB_HUMAN | beta-casein | X | |
| 135 | IESL [symbol] SSEESITEYK | 1 | sp\|P05814\|CASB_HUMAN | beta-casein | X | |
| 106 | ESLSSSEESITEYK | 1 | sp\|P05814\|CASB_HUMAN | beta-casein | X | |
| 135 | IESLSS [symbol] EESITEYK | 1 | sp\|P05814\|CASB_HUMAN | beta-casein | X | |
| 147 | LLLNPTHQIYPVTQ | | sp\|P05814\|CASB_HUMAN | beta-casein | X | |
| 272 | TIESLSSSEESITEYK | 1 | sp\|P05814\|CASB_HUMAN | beta-casein | X | |
| 214 | QKVEKVKHEDQQQGEDEHQD | | sp\|P05814\|CASB_HUMAN | beta-casein | X | |
| 236 | RETIESLSSSEESITEYKQK | 1 | sp\|P05814\|CASB_HUMAN | beta-casein | X | |
| 103 | ENLHLPLPLLQ | | sp\|P05814\|CASB_HUMAN | beta-casein | X | |
| 113 | ETIESLSSSEESITEYKQKVEK | 1 | sp\|P05814\|CASB_HUMAN | beta-casein | X | |
| 272 | TIESLSSSEESITEYK | 2 | sp\|P05814\|CASB_HUMAN | beta-casein | X | |
| 193 | PAVVLPVPQPEIMEVPKAK | | sp\|P05814\|CASB_HUMAN | beta-casein | X | |
| 235 | RETIESLSSSEESITEYKQ | 2 | sp\|P05814\|CASB_HUMAN | beta-casein | X | |
| 110 | ETIESLSSSEESITEYK | 2 | sp\|P05814\|CASB_HUMAN | beta-casein | X | |
| 206 | PVPQPEI | | sp\|P05814\|CASB_HUMAN | beta-casein | X | |
| 237 | RETIESLS [symbol] EESITEYKQKVE | 2 | sp\|P05814\|CASB_HUMAN | beta-casein | X | |
| 298 | VMPVLKSPTIP | | sp\|P05814\|CASB_HUMAN | beta-casein | X | |
| 112 | ETIESLSSSEESITEYKQK | | sp\|P05814\|CASB_HUMAN | beta-casein | X | |
| 234 | RETIESLSSSEESITEYK | 1 | sp\|P05814\|CASB_HUMAN | beta-casein | X | |
| 243 | [symbol] EESITE | 1 | sp\|P05814\|CASB_HUMAN | beta-casein | X | |
| 234 | RETIE [symbol] LSSSEESITEYK | 2 | sp\|P05814\|CASB_HUMAN | beta-casein | X | |
| 110 | ETIESLSSSEESITEYK | 1 | sp\|P05814\|CASB_HUMAN | beta-casein | X | |
| 113 | ETIESLSSSEESITEYKQKVEK | 1 | sp\|P05814\|CASB_HUMAN | beta-casein | X | |
| 106 | ESLSSSEESITEYK | 2 | sp\|P05814\|CASB_HUMAN | beta-casein | X | |
| 124 | GRVMPVLK | | sp\|P05814\|CASB_HUMAN | beta-casein | X | |
| 182 | NLHLPLP | | sp\|P05814\|CASB_HUMAN | beta-casein | X | |
| 185 | NQELLLNPT | | sp\|P05814\|CASB_HUMAN | beta-casein | X | |
| 234 | RETIESLSSSEESITEYK | 2 | sp\|P05814\|CASB_HUMAN | beta-casein | X | |
| 129 | HEDQQQGEDEHQDK | | sp\|P05814\|CASB_HUMAN | beta-casein | X | |
| 175 | LPVPQPEIM | | sp\|P05814\|CASB_HUMAN | beta-casein | X | |
| 112 | ETIESLSSSEESITEYKQK | 1 | sp\|P05814\|CASB_HUMAN | beta-casein | X | |
| 118 | FDPQIPKL | | sp\|P05814\|CASB_HUMAN | beta-casein | X | |
| 132 | HLPLPLL | | sp\|P05814\|CASB_HUMAN | beta-casein | X | |
| 242 | RETIESLSSSEESITEYKQKVEKVKHEDQQQG | 2 | sp\|P05814\|CASB_HUMAN | beta-casein | X | |
| 178 | LSSSEESITEYK | 1 | sp\|P05814\|CASB_HUMAN | beta-casein | X | |
| 205 | PVHNPISV | | sp\|P05814\|CASB_HUMAN | beta-casein | X | |
| 309 | VVLPVPQPEIMEVPKAK | | sp\|P05814\|CASB_HUMAN | beta-casein | X | |
| 194 | PAVVLPVPQPEIMEVPKAKDTVYTKGR | | sp\|P05814\|CASB_HUMAN | beta-casein | X | |
| 237 | RETIESLSSSEESITEYKQKVE | 2 | sp\|P05814\|CASB_HUMAN | beta-casein | X | |
| 237 | RETIESL [symbol] SSEESITEYKQKVE | 2 | sp\|P05814\|CASB_HUMAN | beta-casein | X | |
| 272 | TIE [symbol] LSSSEESITEYK | 1 | sp\|P05814\|CASB_HUMAN | beta-casein | X | |
| 254 | SSEESITE | 1 | sp\|P05814\|CASB_HUMAN | beta-casein | X | |
| 109 | ETIESLSSSEESITEY | 1 | sp\|P05814\|CASB_HUMAN | beta-casein | X | |
| 110 | ETIESLSSSEESITEYK | 1 | sp\|P05814\|CASB_HUMAN | beta-casein | X | |
| 234 | RETIESL [symbol] SSEESITEYK | 2 | sp\|P05814\|CASB_HUMAN | beta-casein | X | |
| 238 | RETIESLSSSEESITEYKQKVEK | 1 | sp\|P05814\|CASB_HUMAN | beta-casein | X | |
| 238 | RETIESLSSSEESITEYKQKVEK | 2 | sp\|P05814\|CASB_HUMAN | beta-casein | X | |
| 260 | SSSEESITEYKQKVEK | 1 | sp\|P05814\|CASB_HUMAN | beta-casein | X | |
| 174 | LPVPQPEI | | sp\|P05814\|CASB_HUMAN | beta-casein | X | |
| 507 | DQQQGEDEHQDKIYP | | sp\|P05814\|CASB_HUMAN | beta-casein | | |
| 508 | EESITEYKQKV | | sp\|P05814\|CASB_HUMAN | beta-casein | | |
| 509 | EVPKAKDTVYTKG | | sp\|P05814\|CASB_HUMAN | beta-casein | | |
| 510 | AQPAVVLPVPQPEIMEVPKAK | | sp\|P05814\|CASB_HUMAN | beta-casein | | |
| 511 | LPVPQPEIMEVPKA | | sp\|P05814\|CASB_HUMAN | beta-casein | | |
| 319 | QLAPIWDKLGETYKDH | 2 | sp\|P07237\|PDIA1_HUMAN | protein disulfide- | | X |
| | | | | isomerase | | |
| 329 | TYYANPAVVRPHAQIPQRQY | | sp\|P07498\|CASK_HUMAN | kappa-casein | | X |
| 323 | LPNSHPPTV | | sp\|P07498\|CASK_HUMAN | kappa-casein | | X |
| 330 | YANPAVVRPHAQIPQR | | sp\|P07498\|CASK_HUMAN | kappa-casein | | X |
| 320 | ANPAVVRPHAQIPQRQY | | sp\|P07498\|CASK_HUMAN | kappa-casein | | X |
| 324 | LPNSHPPTVVR | | sp\|P07498\|CASK_HUMAN | kappa-casein | X | X |
| 326 | TYYANPAVVRPHA | | sp\|P07498\|CASK_HUMAN | kappa-casein | X | X |
| 328 | TYYANPAVVRPHAQIPQR | | sp\|P07498\|CASK_HUMAN | kappa-casein | X | X |
| 327 | TYYANPAVVRPHAQIP | | sp\|P07498\|CASK_HUMAN | kappa-casein | X | |
| 334 | DDPDAPLQPVTPLQLFEGRRN | | sp\|P0C0L4\|CO4A_HUMAN | complement C4-A | X | X |
| 357 | RPDIQYPDATDEDITSHME [symbol] EELNGAYK | 1 | sp\|P10451\|OSTP_HUMAN | osteopontin | | X |
| 357 | RPDIQYPDATDEDIT [symbol] HMESEELNGAYK | 1 | sp\|P10451\|OSTP_HUMAN | osteopontin | | X |
| 339 | ATDEDITSH | | sp\|P10451\|OSTP_HUMAN | osteopontin | | X |
| 343 | E [symbol] EELNGAYK | 1 | sp\|P10451\|OSTP_HUMAN | osteopontin | | X |
| 349 | IPVKQAD [symbol] G | 1 | sp\|P10451\|OSTP_HUMAN | osteopontin | | X |
| 366 | TYDGRGDSVVYGLR | | sp\|P10451\|OSTP_HUMAN | osteopontin | | X |
| 344 | GDSVVYGLR | | sp\|P10451\|OSTP_HUMAN | osteopontin | | X |
| 356 | RPDIQYPDA [symbol] DEDITSH | 1 | sp\|P10451\|OSTP_HUMAN | osteopontin | | X |
| 352 | RI [symbol] HELDSASSEVN | 1 | sp\|P10451\|OSTP_HUMAN | osteopontin | | X |
| 337 | AIPVAQDLNAPS | | sp\|P10451\|OSTP_HUMAN | osteopontin | | X |
| 362 | [symbol] HELDSA [symbol] SEVN | 2 | sp\|P10451\|OSTP_HUMAN | osteopontin | | X |
| 338 | AIPVAQDLNAPSD | | sp\|P10451\|OSTP_HUMAN | osteopontin | | X |
| 360 | RRPDIQYPDATDEDITSHMESEELNGAYK | 1 | sp\|P10451\|OSTP_HUMAN | osteopontin | | X |
| 362 | [symbol] HELDSAS [symbol] EVN | 2 | sp\|P10451\|OSTP_HUMAN | osteopontin | | X |
| 362 | SHELD [symbol] ASSEVN | 1 | sp\|P10451\|OSTP_HUMAN | osteopontin | | X |
| 341 | DQ [symbol] AETHSHKQSRLY | 1 | sp\|P10451\|OSTP_HUMAN | osteopontin | | X |
| 342 | EDITSHME | | sp\|P10451\|OSTP_HUMAN | osteopontin | | X |
| 350 | I [symbol] HELDSA [symbol] SEVN | 2 | sp\|P10451\|OSTP_HUMAN | osteopontin | | X |
| 345 | HELD [symbol] ASSEVN | 1 | sp\|P10451\|OSTP_HUMAN | osteopontin | | X |
| 340 | DIQYPDATDEDITSH | | sp\|P10451\|OSTP_HUMAN | osteopontin | X | X |
| 350 | ISHELD [symbol] ASSEVN | 1 | sp\|P10451\|OSTP_HUMAN | osteopontin | X | X |
| 356 | RPDIQYPDATDEDITSH | | sp\|P10451\|OSTP_HUMAN | osteopontin | X | X |
| 358 | RRPDIQYPDATDEDIT | | sp\|P10451\|OSTP_HUMAN | osteopontin | X | X |
| 359 | RRPDIQYPDA [symbol] DEDITSH | 1 | sp\|P10451\|OSTP_HUMAN | osteopontin | X | X |
| 360 | RRPDIQYPDATDEDITSHME [symbol] EELNGAYK | 1 | sp\|P10451\|OSTP_HUMAN | osteopontin | X | X |
| 361 | [symbol] EELNGAYK | 1 | sp\|P10451\|OSTP_HUMAN | osteopontin | X | X |
| 364 | SKSKKFRRPDIQYPDA [symbol] DEDITSH | 1 | sp\|P10451\|OSTP_HUMAN | osteopontin | X | X |
| 364 | SKSKKFRRPDIQYPDATDEDITSH | | sp\|P10451\|OSTP_HUMAN | osteopontin | X | X |
| 355 | RPDIQYPDATDEDIT | | sp\|P10451\|OSTP_HUMAN | osteopontin | X | |
| 360 | RRPDIQYPDATDEDITSHMESEELNGAYK | 1 | sp\|P10451\|OSTP_HUMAN | osteopontin | X | |
| 365 | SKSKKFRRPDIQYPDATDEDITSHMESEELNGAYK | 1 | sp\|P10451\|OSTP_HUMAN | osteopontin | X | |
| 359 | RRPDIQYPDATDEDITSH | | sp\|P10451\|OSTP_HUMAN | osteopontin | X | |
| 344 | GDSVVYGLR | | sp\|P10451\|OSTP_HUMAN | osteopontin | X | |
| 359 | RRPDIQYPDATDEDITSH | 1 | sp\|P10451\|OSTP_HUMAN | osteopontin | X | |
| 365 | SKSKKFRRPDIQYPDA [symbol] DEDITSHMESEEL | 2 | sp\|P10451\|OSTP_HUMAN | osteopontin | X | |
| | NGAYK | | | | | |
| 350 | ISHELDSASSEVN | 1 | sp\|P10451\|OSTP_HUMAN | osteopontin | X | |
| 365 | SKSKKFRRPDIQYPDATDEDITSHMESEELNGAYK | 1 | sp\|P10451\|OSTP_HUMAN | osteopontin | X | |
| 351 | NKYPDAVAT | | sp\|P10451\|OSTP_HUMAN | osteopontin | X | |
| 360 | RRPDIQYPDATDEDITSHMESEELNGAYK | 2 | sp\|P10451\|OSTP_HUMAN | osteopontin | X | |
| 363 | SKSKKFRRPDIQYPDATD | | sp\|P10451\|OSTP_HUMAN | osteopontin | X | |
| 365 | SKSKKFRRPDIQYPDA [symbol] DEDITSHME | 2 | sp\|P10451\|OSTP_HUMAN | osteopontin | X | |
| | [symbol] EELNGAYK | | | | | |
| 516 | ATDEDITSHMESEELNGAYK | | sp\|P10451\|OSTP_HUMAN | osteopontin | | |
| 517 | EDITSHMESEELNGAYK | | sp\|P10451\|OSTP_HUMAN | osteopontin | | |
| 518 | DIQYPDATDEDITSHMESEELNGAYK | | sp\|P10451\|OSTP_HUMAN | osteopontin | | |
| 519 | DDQSAETHSHKQSRLY | | sp\|P10451\|OSTP_HUMAN | osteopontin | | |
| 371 | SPYEKVSAGNGGSSLS | | sp\|P15941\|MUC1_HUMAN | mucin-1 | | X |
| 376 | TNPAVAATSANL | | sp\|P15941\|MUC1_HUMAN | mucin-1 | | X |
| 372 | STDRSPYEKVSAGNGGSSLSY | | sp\|P15941\|MUC1_HUMAN | mucin-1 | | X |
| 369 | SPYEKVSAGNGGSS | | sp\|P15941\|MUC1_HUMAN | mucin-1 | X | X |
| 370 | SPYEKVSAGNGGSSL | | sp\|P15941\|MUC1_HUMAN | mucin-1 | X | X |
| 374 | TDRSPYEKVSAGNGGSSLSY | | sp\|P15941\|MUC1_HUMAN | mucin-1 | X | X |
| 375 | TDRSPYEKVSAGNGGSSLSYTNPAVAATSANL | | sp\|P15941\|MUC1_HUMAN | mucin-1 | X | X |
| 368 | DRSPYEKVSAGNGGSSLS | | sp\|P15941\|MUC1_HUMAN | mucin-1 | X | |
| 373 | TDRSPYEKVSAGNGGSSLS | | sp\|P15941\|MUC1_HUMAN | mucin-1 | X | |
| 377 | SGNHPITVHCSAGAGRTGTFCALSTV | | sp\|P18433\|PTPRA_HUMAN | receptor-type | X | |
| | | | | tyrosine-protein | | |
| | | | | phosphatase alpha | | |
| 378 | EGGFVEGVNK | | sp\|P19835\|CELL_HUMAN | bile salt-activated | X | X |
| | | | | lipase | | |
| 379 | KLGAVYTEGGFVEGVNK | | sp\|P19835\|CEL_HUMAN | bile salt-activated | X | |
| | | | | lipase | | |
| 380 | RQKASLTNVTDPSLDLTSLSLEVGCGAPAPV | 1 | sp\|P22079\|PERL_HUMAN | lactoperoxidase | | X |
| 380 | RQKASLTNVTDPSLDLTSLSLEVGCGAPAPV | 1 | sp\|P22079\|PERL_HUMAN | lactoperoxidase | | X |
| 382 | DPSKPSSNVAGVVIIV | | sp\|P22897\|MRC1_HUMAN | macrophage mannose | X | |
| | | | | receptor 1 | | |
| 381 | DPSKPSSNVAGVVII | | sp\|P22897\|MRC1_HUMAN | macrophage mannose | X | |
| | | | | receptor 1 | | |
| 404 | RLQNPSE [symbol] SEPIPLESREEYMNGMN | 1 | sp\|P47710\|CASA1_HUMAN | alpha-S1-casein | | X |
| 401 | RLQNPSESSEPIPLE | | sp\|P47710\|CASA1_HUMAN | alpha-S1-casein | | X |
| 404 | RLQNP [symbol] ESSEPIPLE [symbol] REEYMNGMN | 2 | sp\|P47710\|CASA1_HUMAN | alpha-S1-casein | | X |
| 408 | RPKLPLRYPERLQ | | sp\|P47710\|CASA1_HUMAN | alpha-S1-casein | | X |
| 396 | NPSESSEPIPLESREEYMNGMN | | sp\|P47710\|CASA1_HUMAN | alpha-S1-casein | | X |
| 405 | RLQNPSESSEPIPLESREEYMNGMNR | 1 | sp\|P47710\|CASA1_HUMAN | alpha-S1-casein | | X |
| 388 | LQNPSESSEPIPLE | | sp\|P47710\|CASA1_HUMAN | alpha-S1-casein | X | X |
| 389 | LQNPSESSEPIPLESR | | sp\|P47710\|CASA1_HUMAN | alpha-S1-casein | X | X |
| 390 | LQNPSESSEPIPLESREEYMNGMN | | sp\|P47710\|CASA1_HUMAN | alpha-S1-casein | X | X |
| 395 | NPSESSEPIPLESR | | sp\|P47710\|CASA1_HUMAN | alpha-S1-casein | X | X |
| 397 | NYEKNNVML | | sp\|P47710\|CASA1_HUMAN | alpha-S1-casein | X | X |
| 400 | RLQNPSESSEPIP | | sp\|P47710\|CASA1_HUMAN | alpha-S1-casein | X | X |
| 402 | RLQNPSESSEPIPLESR | | sp\|P47710\|CASA1_HUMAN | alpha-S1-casein | X | X |
| 403 | RLQNPSESSEPIPLESREEYMNGM | | sp\|P47710\|CASA1_HUMAN | alpha-S1-casein | X | X |
| 404 | RLQNPSESSEPIPLE [symbol] REEYMNGMN | 1 | sp\|P47710\|CASA1_HUMAN | alpha-S1-casein | X | X |
| 404 | RLQNPSESSEPIPLESREEYMNGMN | | sp\|P47710\|CASA1_HUMAN | alpha-S1-casein | X | X |
| 404 | RLQNPSESSEPIPLE [symbol] REEYMNGMN | 2 | sp\|P47710\|CASA1_HUMAN | alpha-S1-casein | X | |
| 405 | RLQNPSESSEPIPLESREEYMNGMNR | | sp\|P47710\|CASA1_HUMAN | alpha-S1-casein | X | |
| 404 | RLQNPSESSEPIPLESREEYMNGMN | 1 | sp\|P47710\|CASA1_HUMAN | alpha-S1-casein | X | |
| 405 | RLQNPSESSEPIPLESREEYMNGMNR | 1 | sp\|P47710\|CASA1_HUMAN | alpha-S1-casein | X | |
| 399 | RLQNPSE | | sp\|P47710\|CASA1_HUMAN | alpha-S1-casein | X | |
| 409 | RPKLPLRYPERLQNPSESSEPIPLESREEYMNGMN | 1 | sp\|P47710\|CASA1_HUMAN | alpha-S1-casein | X | |
| 405 | RLQNPSESSEPIPLESREEYMNGMNR | 2 | sp\|P47710\|CASA1_HUMAN | alpha-S1-casein | X | |
| 398 | QRNILREKQTDEIKDTR | | sp\|P47710\|CASA1_HUMAN | alpha-S1-casein | X | |
| 394 | NPSESSEPIP | | sp\|P47710\|CASA1_HUMAN | alpha-S1-casein | X | |
| 422 | TKIIEGGAAHKDGRLQ | | sp\|P78352\|DLG4_HUMAN | disks large homolog 4 | X | |
| 429 | DGPERVTVIANAQDLS | | sp\|Q13410\|BT1A1_HUMAN | butyrophilin | X | X |
| | | | | subfamily 1 member | | |
| | | | | A1 | | |
| 431 | DGREQEAEQMPEYR | | sp\|Q13410\|BT1A1_HUMAN | butyrophilin | X | X |
| | | | | subfamily 1 member | | |
| | | | | A1 | | |
| 433 | DGREQEAEQMPEYRGR | | sp\|Q13410\|BT1A1_HUMAN | butyrophilin | X | X |
| | | | | subfamily 1 member | | |
| | | | | A1 | | |
| 437 | EIPLSPMGEDSAPRDADTLH | | sp\|Q13410\|BT1A1_HUMAN | butyrophilin | X | X |
| | | | | subfamily 1 member | | |
| | | | | A1 | | |
| 440 | GREQEAEQMPEYR | | sp\|Q13410\|BT1A1_HUMAN | butyrophilin | X | X |
| | | | | subfamily 1 member | | |
| | | | | A1 | | |
| 444 | IPLSPMGEDSAPRDADTLH | | sp\|Q13410\|BT1A1_HUMAN | butyrophilin | X | X |
| | | | | subfamily 1 member | | |
| | | | | A1 | | |
| 445 | KEIPLSPMGED | | sp\|Q13410\|BT1A1_HUMAN | butyrophilin | X | X |
| | | | | subfamily 1 member | | |
| | | | | A1 | | |
| 446 | KEIPLSPMGEDSAPR | | sp\|Q13410\|BT1A1_HUMAN | butyrophilin | X | X |
| | | | | subfamily 1 member | | |
| | | | | A1 | | |
| 447 | KEIPLSPMGEDSAPRDADT | | sp\|Q13410\|BT1A1_HUMAN | butyrophilin | X | X |
| | | | | subfamily 1 member | | |
| | | | | A1 | | |
| 450 | KEIPLSPMGEDSAPRDADTLHSK | | sp\|Q13410\|BT1A1_HUMAN | butyrophilin | X | X |
| | | | | subfamily 1 member | | |
| | | | | A1 | | |
| 456 | SKLIPTQPSQGAP | | sp\|Q13410\|BT1A1_HUMAN | butyrophilin | X | X |
| | | | | subfamily 1 member | | |
| | | | | A1 | | |
| 430 | DGREQEAEQMPEY | | sp\|Q13410\|BT1A1_HUMAN | butyrophilin | | X |
| | | | | subfamily 1 member | | |
| | | | | A1 | | |
| 436 | EIPLSPMGEDSAPR | | sp\|Q13410\|BT1A1_HUMAN | butyrophilin | | X |
| | | | | subfamily 1 member | | |
| | | | | A1 | | |
| 439 | GRATLVQDGIAKGRVA | | sp\|Q13410\|BT1A1_HUMAN | butyrophilin | | X |
| | | | | subfamily 1 member | | |
| | | | | A1 | | |
| 441 | GREQEAEQMPEYRGR | | sp\|Q13410\|BT1A1_HUMAN | butyrophilin | | X |
| | | | | subfamily 1 member | | |
| | | | | A1 | | |
| 454 | QDLSKEIPLSPMGEDSAPRDADTLH | | sp\|Q13410\|BT1A1_HUMAN | butyrophilin | | X |
| | | | | subfamily 1 member | | |
| | | | | A1 | | |
| 455 | SKLIPTQPSQG | | sp\|Q13410\|BT1A1_HUMAN | butyrophilin | | X |
| | | | | subfamily 1 member | | |
| | | | | A1 | | |
| 457 | SPMGEDSAPRDADTLH | | sp\|Q13410\|BT1A1_HUMAN | butyrophilin | | X |
| | | | | subfamily 1 member | | |
| | | | | A1 | | |
| 458 | TLVQDGIAK | | sp\|Q13410\|BT1A1_HUMAN | butyrophilin | | X |
| | | | | subfamily 1 member | | |
| | | | | A1 | | |
| 459 | TLVQDGIAKGRVA | | sp\|Q13410\|BT1A1_HUMAN | butyrophilin | | X |
| | | | | subfamily 1 member | | |
| | | | | A1 | | |
| 426 | ADTLHSKLIPTQPSQGAP | | sp\|Q13410\|BT1A1_HUMAN | butyrophilin | X | |
| | | | | subfamily 1 member | | |
| | | | | A1 | | |
| 432 | DGREQEAEQMPEYRG | | sp\|Q13410\|BT1A1_HUMAN | butyrophilin | X | |
| | | | | subfamily 1 member | | |
| | | | | A1 | | |
| 438 | GRATLVQDGIAK | | sp\|Q13410\|BT1A1_HUMAN | butyrophilin | X | |
| | | | | subfamily 1 member | | |
| | | | | A1 | | |
| 442 | IPLSPMGEDS | | sp\|Q13410\|BT1A1_HUMAN | butyrophilin | X | |
| | | | | subfamily 1 member | | |
| | | | | A1 | | |
| 448 | KEIPLSPMGEDSAPRDADTLH | | sp\|Q13410\|BT1A1_HUMAN | butyrophilin | X | |
| | | | | subfamily 1 member | | |
| | | | | A1 | | |
| 449 | KEIPLSPMGEDSAPRDADTLHS | | sp\|Q13410\|BT1A1_HUMAN | butyrophilin | X | |
| | | | | subfamily 1 member | | |
| | | | | A1 | | |
| 452 | KEIPLSPMGEDSAPRDADTLHSKLIPTQPSQGAP | | sp\|Q13410\|BT1A1_HUMAN | butyrophilin | X | |
| | | | | subfamily 1 member | | |
| | | | | A1 | | |
| 461 | LPSKTPPPPPPKTTR | 1 | sp\|Q14185\|DOCK1_HUMAN | dedicator of | X | |
| | | | | cytokinesis protein 1 | | |
| 466 | EKLSALKISN | | sp\|Q659A1\|NARG2_HUMAN | NMDA receptor- | X | |
| | | | | regulated protein 2 | | |
| 467 | KVNMISREQFDTLTPEPP | | sp\|Q6PKG0\|LARP1_HUMAN | la-related protein 1 | X | |
| 470 | TKVGEIFSAAGAAF | | sp\|Q8IXM2\|BAP18_HUMAN | chromatin complexes | X | |
| | | | | subunit BAP18 | | |
| 503 | SPPPPPPPP | | sp\|Q8IZP0\|ABI1_HUMAN | Abl interactor 1 | X | |
| 472 | QRTSSIATALNTSGAGGSRP | 1 | sp\|Q8N4C8\|MINK1_HUMAN | misshapen-like | X | |
| | | | | kinase 1 | | |
| 472 | QRTSSIATALNTSGAGGSRP | 1 | sp\|Q8N4C8\|MINK1_HUMAN | misshapen-like | X | |
| | | | | kinase 1 | | |
| 477 | DLLVEILMRPTIS | 1 | sp\|Q8TEL6\|TP4AP_HUMAN | short transient | X | |
| | | | | receptor potential | | |
| | | | | channel 4-associated | | |
| | | | | protein | | |
| 486 | LPIIQKLEPQ | | sp\|Q99541\|PLIN2_HUMAN | perilipin-2 | X | X |
| 487 | LPIIQKLEPQIA | | sp\|Q99541\|PLIN2_HUMAN | perilipin-2 | X | X |
| 483 | AEMDKSSQETQRSEHKTH | | sp\|Q99541\|PLIN2_HUMAN | perilipin-2 | | X |
| 484 | DQGAEMDKSSQETQRSEHKTH | | sp\|Q99541\|PLIN2_HUMAN | perilipin-2 | | X |
| 485 | EMDKSSQETQRSEHKTH | | sp\|Q99541\|PLIN2_HUMAN | perilipin-2 | | X |
| 491 | WGRGNFTEGKVPH | | sp\|Q9BYT3\|STK33_HUMAN | serine/threonine- | X | |
| | | | | protein kinase 33 | | |
| 497 | TVLGNGSSLSLPEGQSLRLVCAV | | sp\|Q9Y336\|SIGL9_HUMAN | Sialic acid-binding | X | |
| | | | | Ig-like lectin 9 | | |
| 498 | TVLGNGSSLSLPEGQSLRLVCAVDAVD | 1 | sp\|Q9Y336\|SIGL9_HUMAN | Sialic acid-binding | X | |
| | | | | Ig-like lectin 9 | | |
| 514 | PDPAKQTDRV | | sp\|Q15262\|PTPRK_HUMAN | Receptor-type | | |
| | | | | tyrosine-protein | | |
| | | | | phosphatase kappa | | |
| 515 | VTAEKAPPPPPP | | sp\|O60346\|PHLP1_HUMAN | PH domain leucine- | | |
| | | | | rich repeat- | | |
| | | | | containing protein | | |
| | | | | phosphatase 1 | | |
| 520 | AKSQTEQTQPLSLSLKPDPLAHLSM | | sp\|Q9NQB0\|TF7L2_HUMAN | Transcription factor | | |
| | | | | 7-like 2 | | |
| 521 | SFRVRASSDGEGTMSRP | | sp\|P35568\|IRS1_HUMAN | Insulin receptor | | |
| | | | | substrate 1 | | |
| 522 | CSSPNDSEHGP | | sp\|Q8WUI4\|HDAC7_HUMAN | Histone deacetylase 7 | | |
| 523 | QWLHTQVGVH | | sp\|Q96JM4\|LRIQ1_HUMAN | Leucine-rich repeat | | |
| | | | | and IQ domain- | | |
| | | | | containing protein 1 | | |
| 524 | LAGDALLSLLAGDLGVEVPSAVPRPTLEPAEQL | | sp\|Q6P531\|GGT6_HUMAN | Gamma- | | |
| | | | | glutamyltransferase 6 | | |
| 525 | EHSESTLNVM | | sp\|P42356\|PI4KA_HUMAN | Phosphatidylinositol | | |
| | | | | 4-kinase alpha | | |
| 526 | GLNYHKRCAFSIPNNCSGARKRRLSSTSLA | | tr\|Q8NCK8\|Q8NCK8_HUMAN | cDNA FLJ38565 fis, | | |
| | | | | clone HCHON2005048, | | |
| | | | | highly similar to | | |
| | | | | Serine/threonine- | | |
| | | | | protein kinase D2 | | |
| | | | | (EC 2.7.11.13) | | |
| 527 | AVSEHQLLHDKGKSIQDLR | | sp\|P12272\|PTHR_HUMAN | Parathyroid hormone- | | |
| | | | | related protein | | |
| 528 | IIIGIGNSGGDLAVEISQTA | | tr\|Q9HA79\|Q9HA79_HUMAN | Flavin containing | | |
| | | | | monooxygenase 5, | | |
| | | | | isoform CRA c | | |
| 529 | THTVTY | | sp\|O75369\|FLNB_HUMAN | Filamin-B | | |
| 530 | GPEAAKSDETAAK | | sp\|P04792\|HSPB1_HUMAN | Heat shock protein | | |
| | | | | beta-1 | | |
| 531 | GGGGGGGGGGGGGGGGEAGAVAPYGYTR | | tr\|Q9UN21\|Q9UN21_HUMAN | Androgen receptor | | |
| 532 | SPPPPPPPP | | sp\|Q8IZP0\|ABI1_HUMAN | Abl interactor 1 | | |
| 533 | PPPLPPPPPP | | sp\|Q96JH7\|VCIP1_HUMAN | Deubiquitinating | | |
| | | | | protein VCIP135 | | |
| 534 | IPPPPPP | | sp\|O60610\|DIAP1_HUMAN | Protein diaphanous | | |
| | | | | homolog 1 | | |
| 535 | YPPPPPPPPP | | sp\|Q92841\|DDX17_HUMAN | Probable ATP- | | |
| | | | | dependent RNA | | |
| | | | | helicase DDX17 | | |

Underlined amino acids are possible phosphorylation sites as determined by MS-GFDB or X!Tandem.
Bolded amino acids are unambiguous phosphorylation sites determined by information obtained by tandem MS in the MS-GFDB or X!Tandem.
Italicized amino acids are identified phosphorylation sites confirmed in literature via Phosphosite and Uniprot.

TABLE 4
Identified peptides with over 57% sequence overlap with known bioactive peptides.

| | | Found | | | |
|---|---|---|---|---|---|
| | | peptide | | | Known |
| SEQ | | protein | Overlapping | | bioactive |
| ID | | of | literature bioactive | Known | peptide |
| NO: | Identified peptides | origin | peptide | activity | origin |
| 324 | LPNSHPPTVVR | human | YQRRPAIAINNPYVPRTYYANPAVVRPHAQ | Antibacterial | human |
| 326 | TYYANPAVVRPHA | κ-casein | IPQRQYLPNSHPPTVVRRPNLHPSF | | milk |
| 327 | TYYANPAVVRPHAQIP | | (SEQ ID NO: 504) | | κ-casein |
| 320 | ANPAVVRPHAQIPQRQY | | | | |
| 323 | LPNSHPPTV | | | | |
| 321 | HPPTVVR | | | | |
| 322 | LPNSHPPT | | | | |
| 328 | TYYANPAVVRPHAQIPQR | | | | |
| 329 | TYYANPAVVRPHAQIPQRQY | | | | |
| 330 | YANPAVVRPHAQIPQR | | | | |
| 127 | GRVMPVLKSPTIPFFDPQIPK | human | QPTIPFFDPQIPK (SEQ ID NO: 505) | Immuno- | human |
| 204 | PTIPFFDPQIPKLTD | β-casein | | modulating | β-casein |
| 251 | SPTIPFFDPQIPK | | | | (105-117) |
| 252 | SPTIPFFDPQIPKL | | | | |
| 253 | SPTIPFFDPQIPKLTD | | | | |
| 117 | FDPQIPK | | | | |
| 128 | GRVMPVLKSPTIPFFDPQIPKLTD | | | | |
| | | human | QELLLNPTHQYPVTQPLAPVHN | Antibacterial | human |
| | | β-casein | PISV (SEQ ID NO: 506) | | β-casein |
| | | | | | (184-210) |
| 82 | AVPVQALLLNQELLLNPTHQIYPVTQPL | | | | |
| | APVHNPISV | | | | |
| 76 | ALLLNQELLLNPTHQIYPVTQPLAPVHNPISV | | | | |
| 101 | ELLLNPTHQIYPVTQPLAPVHNPISV | | | | |
| 99 | ELLLNPTHQIYPVT | | | | |
| 100 | ELLLNPTHQIYPVTQ | | | | |
| 134 | HQIYPVTQPLAPVHNPISV | | | | |
| 146 | LLLNPTHQIYPVT | | | | |
| 147 | LLLNPTHQIYPVTQ | | | | |
| 148 | LLLNPTHQIYPVTQPLAP | | | | |
| 149 | LLLNPTHQIYPVTQPLAPVH | | | | |
| 150 | LLLNPTHQIYPVTQPLAPVHNPISV | | | | |
| 152 | LLLNQELLLNPTHQIYPVTQPLAPVHNPISV | | | | |
| 154 | LLNPTHQIYPVTQPLAPVH | | | | |
| 155 | LLNPTHQIYPVTQPLAPVHNPIS | | | | |
| 156 | LLNPTHQIYPVTQPLAPVHNPISV | | | | |
| 158 | LLNQELLLNPTHQIYPVT | | | | |
| 159 | LLNQELLLNPTHQIYPVTQ | | | | |
| 160 | LLNQELLLNPTHQIYPVTQPLAPVHNPISV | | | | |
| 157 | LLNQELLLNPTHQ | | | | |
| 164 | LNPTHQIYPVTQ | | | | |
| | | human | QELLLNPTHQYPVTQPLAPVHN | Antibacterial | human |
| | | β-casein | PISV (SEQ ID NO: 506) | | β-casein |
| | | | | | (184-210) |
| 165 | LNPTHQIYPVTQPLAPVHNPISV | | | | |
| 166 | LNQELLLNPT | | | | |
| 167 | LNQELLLNPTHQ | | | | |
| 169 | LNQELLLNPTHQIYPVTQPLAPVHNPISV | | | | |
| 185 | NQELLLNPT | | | | |
| 186 | NQELLLNPTHQIYP | | | | |
| 187 | NQELLLNPTHQIYPVT | | | | |
| 188 | NQELLLNPTHQIYPVTQ | | | | |
| 189 | NQELLLNPTHQIYPVTQPLAPVH | | | | |
| 190 | NQELLLNPTHQIYPVTQPLAPVHNPISV | | | | |
| 208 | QELLLNPTHQIYP | | | | |
| 209 | QELLLNPTHQIYPVT | | | | |
| 210 | QELLLNPTHQIYPVTQPLAPVHNPISV | | | | |
| 317 | YPVTQPLAPVH | | | | |
| 318 | YPVTQPLAPVHNPISV | | | | |
| 183 | NPTHQIYPVTQ | | | | |
| 184 | NPTHQIYPVTQPLAPVHNPISV | | | | |
| 197 | PLAPVHNPISV | | | | |
| 203 | PTHQIYPVTQPLAPVHNPISV | | | | |
| 205 | PVHNPISV | | | | |
| 207 | PVTQPLAPVHNPISV | | | | |
| 224 | QPLAPVHNPISV | | | | |
| | | human | QELLLNPTHQYPVTQPLAPVHN | Antibacterial | human |
| | | β-casein | PISV (SEQ ID NO: 506) | | β-casein |
| | | | | | (184-210) |
| 269 | THQIYPVTQPLAPVHNPISV | | | | |
| 275 | TQPLAPVHNPISV | | | | |
| 305 | VTQPLAPVHNPISV | | | | |
| 77 | APVHNPISV | | | | |
| 211 | QIYPVTQPLAPVHNPISV | | | | |
| 137 | IYPVTQPLAPVHNPISV | | | | |
| 168 | LNQELLLNPTHQIYPVT | | | | |
| 223 | QPLAPVH | | | | |
| 146 | LLLNPTHQIYPVT | | | | |
| 148 | LLLNPTHQIYPVTQPLAP | | | | |
| 145 | LLLNPTHQIYP | | | | |
| 99 | ELLLNPTHQIYPVT | | | | |
| 159 | LLNQELLLNPTHQIYPVTQ | | | | |

Identified peptides with over 57% sequence overlap with known bioactive peptides.
The overlap between breast milk peptides and the literature peptide is indicated in bolded amino acids.
An amino acid mismatch between the literature peptide and our set of peptides is indicated by an underlined amino acid.
Insertion of an amino acid is indicated by double-underlining.
See, e.g., Liepke, et al., Journal of Chromatography. B, Analytical technologies in the biomedical and life sciences (2001) 752(2): 369-377 (human milk κ-casein); Azuma, et al., Agricultural and Biological Chemistry (1989) 53(10): 2631-2634 (human β-casein (105-117)); Hayes, et al., Biotechnology Journal (2007) 2(4): 435-449 (human β-casein (184-210)).

Antimicrobial Assays.

For E. coli, all three plates clearly show that growth was inhibited by the 6 μg/μL concentration of milk peptides (See, Example 2 and FIGS. 2A-C). In FIG. 2B, the area around B2 the well loaded with 6 μg/μL of milk peptides shows no bacterial growth. This lack of growth demonstrates that these peptides are antimicrobial. The lower concentrations of milk peptides had no effect on inhibition of E. coli.

The microplate assay further showed that these milk peptides inhibited the growth of S. aureus at 8 μg/μL (See, Example 1 and FIGS. 1A-C). The well loaded with 4 μg/μL of milk peptides did not exhibit growth inhibition for S. aureus.

### Conclusions

This study demonstrated a novel and successful approach for the identification of peptides from human milk. Ferranti et al. (Ferranti, et al., J. Dairy Res. (2004) 71(1):74-87) used three different mass spectrometers and Edman sequencing to determine the sequence of naturally occurring peptides in human milk, whereas the present study employed a single mass spectrometer with automated tandem mass spectrometry. The analytical technique used identified smaller peptides than those identified by a 2D gel method, although the two methods yield complementary information (Armaforte, et al., Int Dairy J (2010) 20(10):715-723).

By putting all identified peptides on the exclusion list for each following round of tandem fragmentation, the number of unique peptides identified increased by nearly 5-fold compared to a single tandem identification run. This strategy is excellent for delving deeper into peptide data, and can be applied to many other molecule types. Similar exclusion list strategies employed for proteomics with offline-LC MALDI MS/MS (Chen, et al., Analytical Chemistry (2005) 77(23):7816-7825; Zerck, et al., Journal of Proteome Research (2009) 8(7):3239-3251) and ESI-MS/MS (Wang, et al., Analytical Chemistry (2008) 80(12):4696-4710; Muntel, et al., Rapid Commun Mass Spec (2012) 26(6):701-709; Voisin, et al., PloS One (2011) 6(1):e16352) increased the number of peptides identified. This technique may be better than dynamic exclusion of precursors (on the fly exclusion within the instrumental settings), as the precursor is often selected at the beginning of the peak, not the apex, resulting in poorer results and less chance of identification.

As a result, more than 500 unique naturally-occurring peptides at 99% confidence were found. Interestingly, no peptides derived from the major human milk proteins—lactoferrin, secretory immunoglobulin A and α-lactalbumin—were present, suggesting either that these proteins have greater resistance to milk enzymes or that there exists a specificity in the hydrolysis mechanism that favors the degradation of certain proteins present in milk over others. A potential protein resistance mechanism may be due to glycosylation and/or a tightly packed tertiary structure. Lactoferrin (Van Berkel, et al., Biochem J (1996) 319(Pt 1); 117; Spik, et al., Advances in Experimental Medicine and Biology (1994) 357:21; Barboza, et al., Mol Cell Proteomics. (2012) June; 11(6):M111.015248) and α-lactalbumin (Picariello, et al., Proteomics (2008) 8(18):3833-3847) are N-glycosylated and sIgA is both N- and O-glycosylated (Pierce-Crétel, et al., Eur. J. Biochem. (1982) 125(2):383-388; Pierce-Crétel, et al., Eur. J. Biochem. (1989) 182(2):457-476; Pierce-Crétel, et al., Eur. J. Biochem. (1984) 139(2):337-349). It has been showed that, for example, N-glycosylated lactoferrin has greater resistance to trypsin than does deglycosylated lactoferrin (van Veen, et al., Eur. J. Biochem. (2004) 271(4):678-684). However, glycosylation alone does not explain which proteins were partially-digested in milk, as many peptides were derived from proteins that are glycosylated. For example, butyrophilin (Picariello, et al., Proteomics (2008) 8(18):3833-3847) is N-glycosylated, kappa-casein (Fiat, et al., Eur. J. Biochem. (1980) 111(2):333-339) is O-glycosylated, and osteopontin (Christensen, et al., Biochem J (2005) 390(Pt 1):285) and mucin-1 (Parry, et al., Glycobiology (2006) 16(7):623-634; Hanisch, et al., Journal of Biological Chemistry (1989) 264(2):872; Hanisch, et al., Glycoconjugate J (1990) 7(6):525-543) are both N- and O-glycosylated.

Of these peptides, 72 were demonstrated to have at least 57% overlap with known bioactive peptides. These results show that pre-digestion of milk proteins within the mammary gland releases potential bioactive peptides with antimicrobial functions. Milk proteases may be specifically releasing bioactive peptides from milk proteins to enhance infant health by preventing bacterial infection.

Peptides isolated from human milk inhibited the growth of E. coli and S. aureus. These naturally-produced milk peptides find use for protecting infection in the infant. Alternatively, the mother may produce these peptides to aid in the prevention and treatment of bacterially-induced mastitis.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1-48. (canceled)

49. A method of reducing, inhibiting or preventing the growth or proliferation of a bacterial organism, comprising contacting the bacterial organism with an antibacterial peptide comprising from 5 to 55 amino acid residues of alpha-S1-casein (CASA1), wherein the peptide comprises or consists essentially of a subsequence of alpha-S1-casein (CASA1) within amino acid positions selected from 16-68, 70-79 and 175-183, wherein the amino acid positions are with reference to UNIPROT code no. P47710.

50-56. (canceled)

57. A method for reducing, preventing, inhibiting and/or mitigating a bacterial infection of the mammary gland in a lactating mammal, comprising administering to a mammary gland of the lactating mammal a therapeutically effective amount of an antibacterial peptide comprising from 5 to 55 amino acid residues of alpha-S1-casein (CASA1), wherein the peptide comprises or consists essentially of a subsequence of alpha-S1-casein (CASA1) within amino acid positions selected from 16-68, 70-79 and 175-183, wherein the amino acid positions are with reference to UNIPROT code no. P47710.

58. A method for reducing, preventing, inhibiting and/or mitigating a bacterial infection in the oral cavity of a nursing mammal, comprising administering to the oral cavity of the nursing mammal a therapeutically effective amount of an antibacterial peptide comprising from 5 to 55 amino acid residues of alpha-S1-casein (CASA1), wherein the peptide comprises or consists essentially of a subsequence of alpha-S1-casein (CASA1) within amino acid positions selected from 16-68, 70-79 and 175-183, wherein the amino acid positions are with reference to UNIPROT code no. P47710.

59-62. (canceled)

63. The method of claim 49, wherein the subsequence or peptide comprises or consists essentially of a subsequence of alpha-S1-casein (CASA1) within amino acid positions 16-68, wherein the amino acid positions are with reference to UNIPROT code no. P47710.

64. The method of claim 49, wherein the CASA1 subsequence or peptide comprises or consists essentially of from 7 to 35 amino acid residues.

65. The method of claim 49, wherein the CASA1 subsequence or peptide comprises or consists essentially of an amino acid sequence selected from the group consisting of RPKLPLR (SEQ ID NO: 406); RLQNPSE (SEQ ID NO: 399); NPSESSEPIP (SEQ ID NO: 394) and NILREKQTDE (SEQ ID NO: 392).

66. The method of claim 49, wherein the CASA1 subsequence or peptide is selected from the group consisting of RPKLPLR (SEQ ID NO: 406); RPKLPLRYPE (SEQ ID NO: 407); RPKLPLRYPERLQ (SEQ ID NO: 408); RPKLPLRYPERLQNPSESSEPIPLESREEYMNGMN (SEQ ID NO: 409); RLQNPSE (SEQ ID NO: 399); RLQNPSESSEPIP (SEQ ID NO: 400); RLQNPSESSEPIPLE (SEQ ID NO: 401); RLQNPSESSEPIPLESR (SEQ ID NO: 402); RLQNPSESSEPIPLESREEYMNGM (SEQ ID NO: 403); RLQNPSESSEPIPLESREEYMNGMN (SEQ ID NO: 404); RLQNPSESSEPIPLESREEYMNGMNR (SEQ ID NO: 405); LQNPSESSEPIPLE (SEQ ID NO: 388); LQNPSESSEPIPLESR (SEQ ID NO: 389); LQNPSESSEPIPLESREEYMNGMN (SEQ ID NO: 390); NPSESSEPIP (SEQ ID NO: 394); NPSESSEPIPLES (SEQ ID NO: 539); NPSESSEPIPLESREEYMNGMN (SEQ ID NO: 396); MNRQRNILR (SEQ ID NO: 391); QRNILREKQTDEIKDTR (SEQ ID NO: 398); NILREKQTDE (SEQ ID NO: 392); NILREKQTDEIKDTR (SEQ ID NO: 393); EKQTDEIKDTR (SEQ ID NO: 387); NYEKNNVML (SEQ ID NO: 397); and YEKNNVML (SEQ ID NO: 410).

67. The method of claim 49, wherein the CASA1 subsequence or peptide is phosphorylated at one or more amino acids.

68. The method of claim 49, wherein the bacterial organism is located within a mammary gland of a lactating mammal.

69. The method of claim 49, wherein the bacterial organism is located within an oral cavity of a mammal.

70. The method of claim 49, wherein the bacterial organism is selected from the group consisting of Staphylococcus aureus and Escherichia coli.

71. The method of claim 49, wherein the CASA1 subsequence or peptide is formulated for topical administration to a mammal.

72. The method of claim 49, further comprising contacting the contacting the bacterial organism with one or more peptides comprising or consisting essentially of a subsequence of a protein selected from the group consisting of: polymeric immunoglobulin receptor (PIGR); beta-casein (CASB); butyrophilin subfamily 1 member A1 (BT1A1); osteopontin (OSTP); mucin-1 (MUC1); perilipin-2 (PLIN2); neural Wiskott-Aldrich syndrome protein (WASL); cancer susceptibility candidate gene 3 protein (CASC3); inositol polyphosphate phosphatase-like 1 (SHIP2); protein diaphanous homolog 1 (DIAP1); ceruloplasmin (CERU); haptoglobin (HPT); complement C3 (CO3); pro-epidermal growth factor (EGF); protein disulfide-isomerase (PDIA1); kappa-casein (CASK); thrombospondin-1 (TSP1); heat shock protein HSP 90-beta (HS90B); complement C4-A (CO4A); receptor-type tyrosine-protein phosphatase alpha (PTPRA); bile salt-activated lipase (CEL); lactoperoxidase (PERL); macrophage mannose receptor 1 (MRC1); tenascin (TENA); xanthine dehydrogenase/oxidase (XDH); paxillin (PAXI); fatty acid synthase (FAS); centromere protein F (CENPF); afadin (AFAD); heterogeneous nuclear ribonucleoprotein K (HNRPK); disks large homolog 4 (DLG4); arginase-2, mitochondrial (ARGI2); tyrosine-protein phosphatase non-receptor type 13 (PTN13); E3 ubiquitin-protein ligase CBL-B (CBLB); protein scribble homolog (SCRIB); dedicator of cytokinesis protein 1 (DOCK1); telomeric repeat-binding factor 2 (TERF2); inverted formin-2 (INF2); programmed cell death protein 4 (PDCD4); E3 ubiquitin-protein ligase UBR4 (UBR4); NMDA receptor-regulated protein 2 (NARG2); 1a-related protein 1 (LARP1); prostate androgen-regulated mucin-like protein 1 (PARM1); ubiquitin carboxyl-terminal hydrolase 51 (UBP51); chromatin complexes subunit BAP18 (BAP18); Armadillo repeat-containing protein 10 (ARM10); misshapen-like kinase 1 (MINK1); protein enabled homolog (ENAH); biorientation of chromosomes in cell division protein 1-like 1 (BD1L1); short transient receptor potential channel 4-associated protein (TP4AP); ankyrin repeat and SAM domain-containing protein 1A (ANS1A); mitogen-activated protein kinase kinase kinase kinase 1 (M4K1); GDP-fucose transporter 1 (FUCT1); E3 ubiquitin-protein ligase UHRF1 (UHRF1); mucin-4 (MUC-4); matrix metalloproteinase-19 (MMP19); serine/threonine-protein kinase 33 (STK33); TR10 and F-actin-binding protein (TARA); apoptotic chromatin condensation inducer in the nucleus (ACINU); UPF0760 protein C2orf29 (CB029); zinc finger protein PLAGL1 (PLAL1); cofilin-2 (COF2); sialic acid-binding Ig-like lectin 9 (SIGL9); protein VPRBP (VPRBP); myosin-4 (MYH4); endoplasmic reticulum mannosyl-oligosaccharide 1,2-alpha-mannosidase (MAN1B1); and cDNA F1157167, highly similar to Etoposide-induced protein 2.4.


SEQUENCE LISTING (572 sequences)

SEQ ID NO: 1 - protein, 14 aa, Homo sapiens
PPSRPSVAVPPPPP

SEQ ID NO: 2 - protein, 9 aa, Homo sapiens
RQAPPPPPP

SEQ ID NO: 3 - protein, 12 aa, Homo sapiens
SRPSVAVPPPPP

SEQ ID NO: 4 - protein, 29 aa, Homo sapiens
PSPEADAPVLGSPEKEEAASEPPAAAPDA

SEQ ID NO: 5 - protein, 14 aa, Homo sapiens
KTLDEVTVTIPHDI

SEQ ID NO: 6 - protein, 13 aa, Homo sapiens
SLPGGTAIPPPPP

SEQ ID NO: 7 - protein, 10 aa, Homo sapiens
GSYKKLVYRE

SEQ ID NO: 8 - protein, 6 aa, Homo sapiens
AGSAFA

SEQ ID NO: 9 - protein, 11 aa, Homo sapiens
ITHRIHWESAS

SEQ ID NO: 10 - protein, 9 aa, Homo sapiens
VLYRIFTVN

SEQ ID NO: 11 - protein, 16 aa, Homo sapiens
KLLSKNPKNPYEESSR

SEQ ID NO: 12 - protein, 18 aa, Homo sapiens
AAPDEKVLDSGFREIENK

SEQ ID NO: 13 - protein, 20 aa, Homo sapiens
ADAAPDEKVLDSGFREIENK

SEQ ID NO: 14 - protein, 28 aa, Homo sapiens
ADTRDQADGSRASVDSGSSEEQGGSSRA

SEQ ID NO: 15 - protein, 17 aa, Homo sapiens
AEEKAVADTRDQADGSR

SEQ ID NO: 16 - protein, 18 aa, Homo sapiens
AIQDPRLFAEEKAVADTR

SEQ ID NO: 17 - protein, 24 aa, Homo sapiens
AIQDPRLFAEEKAVADTRDQADGS

SEQ ID NO: 18 - protein, 24 aa, Homo sapiens
ASVDSGSSEEQGGSSRALVSTLVP

SEQ ID NO: 19 - protein, 26 aa, Homo sapiens
ASVDSGSSEEQGGSSRALVSTLVPLG

SEQ ID NO: 20 - protein, 10 aa, Homo sapiens
AVADTRDQAD

SEQ ID NO: 21 - protein, 11 aa, Homo sapiens
AVADTRDQADG

SEQ ID NO: 22 - protein, 12 aa, Homo sapiens
AVADTRDQADGS

SEQ ID NO: 23 - protein, 15 aa, Homo sapiens
AVADTRDQADGSRAS

SEQ ID NO: 24 - protein, 17 aa, Homo sapiens
AVADTRDQADGSRASVD

SEQ ID NO: 25 - protein, 19 aa, Homo sapiens
AVADTRDQADGSRASVDSG

SEQ ID NO: 26 - protein, 25 aa, Homo sapiens
AVADTRDQADGSRASVDSGSSEEQG

SEQ ID NO: 27 - protein, 26 aa, Homo sapiens
AVADTRDQADGSRASVDSGSSEEQGG

SEQ ID NO: 28 - protein, 28 aa, Homo sapiens
AVADTRDQADGSRASVDSGSSEEQGGSS

SEQ ID NO: 29 - protein, 30 aa, Homo sapiens
AVADTRDQADGSRASVDSGSSEEQGGSSRA

SEQ ID NO: 30 - protein, 31 aa, Homo sapiens
AVADTRDQADGSRASVDSGSSEEQGGSSRAL

SEQ ID NO: 31 - protein, 34 aa, Homo sapiens
AVADTRDQADGSRASVDSGSSEEQGGSSRALVST

SEQ ID NO: 32 - protein, 37 aa, Homo sapiens
AVADTRDQADGSRASVDSGSSEEQGGSSRALVSTLVP

SEQ ID NO: 33 - protein, 39 aa, Homo sapiens
AVADTRDQADGSRASVDSGSSEEQGGSSRALVSTLVPLG

SEQ ID NO: 34 - protein, 19 aa, Homo sapiens
DAAPDEKVLDSGFREIENK

SEQ ID NO: 35 - protein, 20 aa, Homo sapiens
DGSRASVDSGSSEEQGGSSR

SEQ ID NO: 36 - protein, 21 aa, Homo sapiens
DGSRASVDSGSSEEQGGSSRA

SEQ ID NO: 37 - protein, 15 aa, Homo sapiens
DPRLFAEEKAVADTR

SEQ ID NO: 38 - protein, 22 aa, Homo sapiens
DQADGSRASVDSGSSEEQGGSS

SEQ ID NO: 39 - protein, 24 aa, Homo sapiens
DQADGSRASVDSGSSEEQGGSSRA

SEQ ID NO: 40 - protein, 25 aa, Homo sapiens
DQADGSRASVDSGSSEEQGGSSRAL

SEQ ID NO: 41 - protein, 27 aa, Homo sapiens
DQADGSRASVDSGSSEEQGGSSRALVS

SEQ ID NO: 42 - protein, 28 aa, Homo sapiens
DQADGSRASVDSGSSEEQGGSSRALVST

SEQ ID NO: 43 - protein, 31 aa, Homo sapiens
DQADGSRASVDSGSSEEQGGSSRALVSTLVP

SEQ ID NO: 44 - protein, 32 aa, Homo sapiens
DQADGSRASVDSGSSEEQGGSSRALVSTLVPL

SEQ ID NO: 45 - protein, 33 aa, Homo sapiens
DQADGSRASVDSGSSEEQGGSSRALVSTLVPLG

SEQ ID NO: 46 - protein, 15 aa, Homo sapiens
DSGSSEEQGGSSRAL

SEQ ID NO: 47 - protein, 16 aa, Homo sapiens
DSGSSEEQGGSSRALV

SEQ ID NO: 48 - protein, 18 aa, Homo sapiens
DSGSSEEQGGSSRALVST

SEQ ID NO: 49 - protein, 21 aa, Homo sapiens
DSGSSEEQGGSSRALVSTLVP

SEQ ID NO: 50 - protein, 22 aa, Homo sapiens
DSGSSEEQGGSSRALVSTLVPL

SEQ ID NO: 51 - protein, 23 aa, Homo sapiens
DSGSSEEQGGSSRALVSTLVPLG

SEQ ID NO: 52 - protein, 14 aa, Homo sapiens
EEKAVADTRDQADG

SEQ ID NO: 53 - protein, 16 aa, Homo sapiens
EEKAVADTRDQADGSR

SEQ ID NO: 54 - protein, 13 aa, Homo sapiens
EKAVADTRDQADG

SEQ ID NO: 55 - protein, 18 aa, Homo sapiens
FAEEKAVADTRDQADGSR

SEQ ID NO: 56 - protein, 35 aa, Homo sapiens
FAEEKAVADTRDQADGSRASVDSGSSEEQGGSSRA

SEQ ID NO: 57 - protein, 21 aa, Homo sapiens
KADAAPDEKVLDSGFREIENK

SEQ ID NO: 58 - protein, 19 aa, Homo sapiens
LFAEEKAVADTRDQADGSR

SEQ ID NO: 59 - protein, 36 aa, Homo sapiens
LFAEEKAVADTRDQADGSRASVDSGSSEEQGGSSRA

SEQ ID NO: 60 - protein, 23 aa, Homo sapiens
QADGSRASVDSGSSEEQGGSSRA

SEQ ID NO: 61 - protein, 16 aa, Homo sapiens
SVDSGSSEEQGGSSRA

SEQ ID NO: 62 - protein, 20 aa, Homo sapiens
SVDSGSSEEQGGSSRALVST

SEQ ID NO: 63 - protein, 23 aa, Homo sapiens
SVDSGSSEEQGGSSRALVSTLVP

SEQ ID NO: 64 - protein, 24 aa, Homo sapiens
SVDSGSSEEQGGSSRALVSTLVPL

SEQ ID NO: 65 - protein, 25 aa, Homo sapiens
SVDSGSSEEQGGSSRALVSTLVPLG

SEQ ID NO: 66 - protein, 26 aa, Homo sapiens
TRDQADGSRASVDSGSSEEQGGSSRA

SEQ ID NO: 67 - protein, 14 aa, Homo sapiens
VADTRDQADGSRAS

SEQ ID NO: 68 - protein, 27 aa, Homo sapiens
VADTRDQADGSRASVDSGSSEEQGGSS

SEQ ID NO: 69 - protein, 29 aa, Homo sapiens
VADTRDQADGSRASVDSGSSEEQGGSSRA

SEQ ID NO: 70 - protein, 15 aa, Homo sapiens
VDSGSSEEQGGSSRA

SEQ ID NO: 71 - protein, 22 aa, Homo sapiens
VDSGSSEEQGGSSRALVSTLVP

SEQ ID NO: 72 - protein, 24 aa, Homo sapiens
VDSGSSEEQGGSSRALVSTLVPLG

SEQ ID NO: 73 - protein, 20 aa, Homo sapiens
YGETAAVYVAVEERKAAGSR

SEQ ID NO: 74 - protein, 16 aa, Homo sapiens
AKDTVYTKGRVMPVLK

SEQ ID NO: 75 - protein, 20 aa, Homo sapiens
ALLLNQELLLNPTHQIYPVT

SEQ ID NO: 76 - protein, 32 aa, Homo sapiens
ALLLNQELLLNPTHQIYPVTQPLAPVHNPISV

SEQ ID NO: 77 - protein, 9 aa, Homo sapiens
APVHNPISV

SEQ ID NO: 78 - protein, 19 aa, Homo sapiens
AQPAVVLPVPQPEIMEVPK

SEQ ID NO: 79 - protein, 26 aa, Homo sapiens
AQPAVVLPVPQPEIMEVPKAKDTVYT

SEQ ID NO: 80 - protein, 27 aa, Homo sapiens
AQPAVVLPVPQPEIMEVPKAKDTVYTK

SEQ ID NO: 81 - protein, 28 aa, Homo sapiens
AQPAVVLPVPQPEIMEVPKAKDTVYTKG

SEQ ID NO: 82 - protein, 37 aa, Homo sapiens
AVPVQALLLNQELLLNPTHQIYPVTQPLAPVHNPISV

SEQ ID NO: 83 - protein, 7 aa, Homo sapiens
DEHQDKI

SEQ ID NO: 84 - protein, 9 aa, Homo sapiens
DEHQDKIYP

SEQ ID NO: 85 - protein, 8 aa, Homo sapiens
DLENLHLP

SEQ ID NO: 86 - protein, 10 aa, Homo sapiens
DLENLHLPLP

SEQ ID NO: 87 - protein, 7 aa, Homo sapiens
DPQIPKL

SEQ ID NO: 88 - protein, 11 aa, Homo sapiens
DPQIPKLTDLE

SEQ ID NO: 89 - protein, 18 aa, Homo sapiens
DPQIPKLTDLENLHLPLP

SEQ ID NO: 90 - protein, 8 aa, Homo sapiens
DTVYTKGR

SEQ ID NO: 91 - protein, 9 aa, Homo sapiens
DTVYTKGRV

SEQ ID NO: 92 - protein, 13 aa, Homo sapiens
DTVYTKGRVMPVL

SEQ ID NO: 93 - protein, 14 aa, Homo sapiens
DTVYTKGRVMPVLK

SEQ ID NO: 94 - protein, 8 aa, Homo sapiens
EESITEYK

SEQ ID NO: 95 - protein, 7 aa, Homo sapiens
EIMEVPK

SEQ ID NO: 96 - protein, 14 aa, Homo sapiens
EIMEVPKAKDTVYT

SEQ ID NO: 97 - protein, 18 aa, Homo sapiens
EKVKHEDQQQGEDEHQDK

SEQ ID NO: 98 - protein, 12 aa, Homo sapiens
ELLLNPTHQIYP

SEQ ID NO: 99 - protein, 14 aa, Homo sapiens
ELLLNPTHQIYPVT

SEQ ID NO: 100 - protein, 15 aa, Homo sapiens
ELLLNPTHQIYPVTQ

SEQ ID NO: 101 - protein, 26 aa, Homo sapiens
ELLLNPTHQIYPVTQPLAPVHNPISV

SEQ ID NO: 102 - protein, 10 aa, Homo sapiens
ENLHLPLPLL

SEQ ID NO: 103 - protein, 11 aa, Homo sapiens
ENLHLPLPLLQ

SEQ ID NO: 104 - protein, 7 aa, Homo sapiens
ESITEYK

SEQ ID NO: 105 - protein, 12 aa, Homo sapiens
ESLSSSEESITE

SEQ ID NO: 106 - protein, 14 aa, Homo sapiens
ESLSSSEESITEYK

SEQ ID NO: 107 - protein, 11 aa, Homo sapiens
ETIESLSSSEE

SEQ ID NO: 108 - protein, 15 aa, Homo sapiens
ETIESLSSSEESITE

SEQ ID NO: 109 - protein, 16 aa, Homo sapiens
ETIESLSSSEESITEY

SEQ ID NO: 110 - protein, 17 aa, Homo sapiens
ETIESLSSSEESITEYK

SEQ ID NO: 111 - protein, 18 aa, Homo sapiens
ETIESLSSSEESITEYKQ

SEQ ID NO: 112 - protein, 19 aa, Homo sapiens
ETIESLSSSEESITEYKQK

SEQ ID NO: 113 - protein, 22 aa, Homo sapiens
ETIESLSSSEESITEYKQKVEK

SEQ ID NO: 114 - protein, 8 aa, Homo sapiens
EVPKAKDT

SEQ ID NO: 115 - protein, 11 aa, Homo sapiens
EVPKAKDTVYT

SEQ ID NO: 116 - protein, 12 aa, Homo sapiens
EVPKAKDTVYTK

SEQ ID NO: 117 - protein, 7 aa, Homo sapiens
FDPQIPK

SEQ ID NO: 118 - protein, 8 aa, Homo sapiens
FDPQIPKL

SEQ ID NO: 119 - protein, 9 aa, Homo sapiens
FDPQIPKLT

SEQ ID NO: 120 - protein, 10 aa, Homo sapiens
FDPQIPKLTD

SEQ ID NO: 121 - protein, 8 aa, Homo sapiens
FFDPQIPK

SEQ ID NO: 122 - protein, 8 aa, Homo sapiens
GEDEHQDK

SEQ ID NO: 123 - protein, 12 aa, Homo sapiens
GEDEHQDKIYPS

SEQ ID NO: 124 - protein, 8 aa, Homo sapiens
GRVMPVLK

SEQ ID NO: 125 - protein, 11 aa, Homo sapiens
GRVMPVLKSPT

SEQ ID NO: 126 - protein, 13 aa, Homo sapiens
GRVMPVLKSPTIP

SEQ ID NO: 127 - protein, 21 aa, Homo sapiens
GRVMPVLKSPTIPFFDPQIPK

SEQ ID NO: 128 - protein, 24 aa, Homo sapiens
GRVMPVLKSPTIPFFDPQIPKLTD

SEQ ID NO: 129 - protein, 14 aa, Homo sapiens
HEDQQQGEDEHQDK

SEQ ID NO: 130 - protein, 17 aa, Homo sapiens
HEDQQQGEDEHQDKIYP

SEQ ID NO: 131 - protein, 18 aa, Homo sapiens
HEDQQQGEDEHQDKIYPS

SEQ ID NO: 132 - protein, 7 aa, Homo sapiens
HLPLPLL

SEQ ID NO: 133 - protein, 6 aa, Homo sapiens
HNPISV

SEQ ID NO: 134 - protein, 19 aa, Homo sapiens
HQIYPVTQPLAPVHNPISV

SEQ ID NO: 135 - protein, 15 aa, Homo sapiens
IESLSSSEESITEYK

SEQ ID NO: 136 - protein, 17 aa, Homo sapiens
IPQQVVPYPQRAVPVQA

SEQ ID NO: 137 - protein, 17 aa, Homo sapiens
IYPVTQPLAPVHNPISV

SEQ ID NO: 138 - protein, 14 aa, Homo sapiens
KDTVYTKGRVMPVL

SEQ ID NO: 139 - protein, 15 aa, Homo sapiens
KDTVYTKGRVMPVLK

SEQ ID NO: 140 - protein, 14 aa, Homo sapiens
KHEDQQQGEDEHQD

SEQ ID NO: 141 - protein, 13 aa, Homo sapiens
KVEKVKHEDQQQG

SEQ ID NO: 142 - protein, 20 aa, Homo sapiens
KVEKVKHEDQQQGEDEHQDK

SEQ ID NO: 143 - protein, 9 aa, Homo sapiens
LENLHLPLP

SEQ ID NO: 144 - protein, 12 aa, Homo sapiens
LENLHLPLPLLQ

SEQ ID NO: 145 - protein, 11 aa, Homo sapiens
LLLNPTHQIYP

SEQ ID NO: 146 - protein, 13 aa, Homo sapiens
LLLNPTHQIYPVT

SEQ ID NO: 147 - protein, 14 aa, Homo sapiens
LLLNPTHQIYPVTQ

SEQ ID NO: 148 - protein, 18 aa, Homo sapiens
LLLNPTHQIYPVTQPLAP

SEQ ID NO: 149 - protein, 20 aa, Homo sapiens
LLLNPTHQIYPVTQPLAPVH

SEQ ID NO: 150 - protein, 25 aa, Homo sapiens
LLLNPTHQIYPVTQPLAPVHNPISV

SEQ ID NO: 151 - protein, 20 aa, Homo sapiens
LLLNQELLLNPTHQIYPVTQ

SEQ ID NO: 152 - protein, 31 aa, Homo sapiens
LLLNQELLLNPTHQIYPVTQPLAPVHNPISV

SEQ ID NO: 153 - protein, 10 aa, Homo sapiens
LLNPTHQIYP

SEQ ID NO: 154 - protein, 19 aa, Homo sapiens
LLNPTHQIYPVTQPLAPVH

SEQ ID NO: 155 - protein, 23 aa, Homo sapiens
LLNPTHQIYPVTQPLAPVHNPIS

SEQ ID NO: 156 - protein, 24 aa, Homo sapiens
LLNPTHQIYPVTQPLAPVHNPISV

SEQ ID NO: 157 - protein, 13 aa, Homo sapiens
LLNQELLLNPTHQ

SEQ ID NO: 158 - protein, 18 aa, Homo sapiens
LLNQELLLNPTHQIYPVT

SEQ ID NO: 159 - protein, 19 aa, Homo sapiens
LLNQELLLNPTHQIYPVTQ

SEQ ID NO: 160 - protein, 30 aa, Homo sapiens
LLNQELLLNPTHQIYPVTQPLAPVHNPISV

SEQ ID NO: 161 - protein, 16 aa, Homo sapiens
LLQPLMQQVPQPIPQT

SEQ ID NO: 162 - protein, 17 aa, Homo sapiens
LLQPLMQQVPQPIPQTL

SEQ ID NO: 163 - protein, 12 aa, Homo sapiens
LMQQVPQPIPQT

SEQ ID NO: 164 - protein, 12 aa, Homo sapiens
LNPTHQIYPVTQ

SEQ ID NO: 165 - protein, 23 aa, Homo sapiens
LNPTHQIYPVTQPLAPVHNPISV

SEQ ID NO: 166 - protein, 10 aa, Homo sapiens
LNQELLLNPT

SEQ ID NO: 167 - protein, 12 aa, Homo sapiens
LNQELLLNPTHQ

SEQ ID NO: 168 - protein, 17 aa, Homo sapiens
LNQELLLNPTHQIYPVT

SEQ ID NO: 169 - protein, 29 aa, Homo sapiens
LNQELLLNPTHQIYPVTQPLAPVHNPISV

SEQ ID NO: 170 - protein, 11 aa, Homo sapiens
LPIPQQVVPYP

SEQ ID NO: 171 - protein, 16 aa, Homo sapiens
LPIPQQVVPYPQRAVP

SEQ ID NO: 172 - protein, 18 aa, Homo sapiens
LPIPQQVVPYPQRAVPVQ

SEQ ID NO: 173 - protein, 19 aa, Homo sapiens
LPIPQQVVPYPQRAVPVQA

SEQ ID NO: 174 - protein, 8 aa, Homo sapiens
LPVPQPEI

SEQ ID NO: 175 - protein, 9 aa, Homo sapiens
LPVPQPEIM

SEQ ID NO: 176 - protein, 10 aa, Homo sapiens
LPVPQPEIME

SEQ ID NO: 177 - protein, 13 aa, Homo sapiens
LPVPQPEIMEVPK

SEQ ID NO: 178 - protein, 12 aa, Homo sapiens
LSSSEESITEYK

SEQ ID NO: 179 - protein, 17 aa, Homo sapiens
LSSSEESITEYKQKVEK

SEQ ID NO: 180 - protein, 15 aa, Homo sapiens
MEVPKAKDTVYTKGR

SEQ ID NO: 181 - protein, 24 aa, Homo sapiens
NILPLAQPAVVLPVPQPEIMEVPK

SEQ ID NO: 182 - protein, 7 aa, Homo sapiens
NLHLPLP

SEQ ID NO: 183 - protein, 11 aa, Homo sapiens
NPTHQIYPVTQ

SEQ ID NO: 184 - protein, 22 aa, Homo sapiens
NPTHQIYPVTQPLAPVHNPISV

SEQ ID NO: 185 - protein, 9 aa, Homo sapiens
NQELLLNPT

SEQ ID NO: 186 - protein, 14 aa, Homo sapiens
NQELLLNPTHQIYP

SEQ ID NO: 187 - protein, 16 aa, Homo sapiens
NQELLLNPTHQIYPVT

SEQ ID NO: 188 - protein, 17 aa, Homo sapiens
NQELLLNPTHQIYPVTQ

SEQ ID NO: 189 - protein, 23 aa, Homo sapiens
NQELLLNPTHQIYPVTQPLAPVH

SEQ ID NO: 190 - protein, 28 aa, Homo sapiens
NQELLLNPTHQIYPVTQPLAPVHNPISV

SEQ ID NO: 191 - protein, 12 aa, Homo sapiens
PAVVLPVPQPEI

SEQ ID NO: 192 - protein, 14 aa, Homo sapiens
PAVVLPVPQPEIME

SEQ ID NO: 193 - protein, 19 aa, Homo sapiens
PAVVLPVPQPEIMEVPKAK

SEQ ID NO: 194 - protein, 27 aa, Homo sapiens
PAVVLPVPQPEIMEVPKAKDTVYTKGR

SEQ ID NO: 195 - protein, 14 aa, Homo sapiens
PIPQQVVPYPQRAV

SEQ ID NO: 196 - protein, 17 aa, Homo sapiens
PIPQQVVPYPQRAVPVQ

SEQ ID NO: 197 - protein, 11 aa, Homo sapiens
PLAPVHNPISV

SEQ ID NO: 198 - protein, 16 aa, Homo sapiens
PLAQPAVVLPVPQPEI

SEQ ID NO: 199 - protein, 14 aa, Homo sapiens
PLMQQVPQPIPQTL

SEQ ID NO: 200 - protein, 8 aa, Homo sapiens
PQIPKLTD

SEQ ID NO: 201 - protein, 12 aa, Homo sapiens
PQIPKLTDLENL

SEQ ID NO: 202 - protein, 10 aa, Homo sapiens
PTHQIYPVTQ

SEQ ID NO: 203 - protein, 21 aa, Homo sapiens
PTHQIYPVTQPLAPVHNPISV

SEQ ID NO: 204 - protein, 15 aa, Homo sapiens
PTIPFFDPQIPKLTD

SEQ ID NO: 205 - protein, 8 aa, Homo sapiens
PVHNPISV

SEQ ID NO: 206 - protein, 7 aa, Homo sapiens
PVPQPEI

SEQ ID NO: 207 - protein, 15 aa, Homo sapiens
PVTQPLAPVHNPISV

SEQ ID NO: 208 - protein, 13 aa, Homo sapiens
QELLLNPTHQIYP

SEQ ID NO: 209 - protein, 15 aa, Homo sapiens
QELLLNPTHQIYPVT

SEQ ID NO: 210 - protein, 27 aa, Homo sapiens
QELLLNPTHQIYPVTQPLAPVHNPISV

SEQ ID NO: 211 - protein, 18 aa, Homo sapiens
QIYPVTQPLAPVHNPISV

SEQ ID NO: 212 - protein, 7 aa, Homo sapiens
QKVEKVK

SEQ ID NO: 213 - protein, 10 aa, Homo sapiens
QKVEKVKHED

SEQ ID NO: 214 - protein, 20 aa, Homo sapiens
QKVEKVKHEDQQQGEDEHQD

SEQ ID NO: 215 - protein, 21 aa, Homo sapiens
QKVEKVKHEDQQQGEDEHQDK

SEQ ID NO: 216 - protein, 13 aa, Homo sapiens
QPAVVLPVPQPEI

SEQ ID NO: 217 - protein, 14 aa, Homo sapiens
QPAVVLPVPQPEIM

SEQ ID NO: 218 - protein, 18 aa, Homo sapiens
QPAVVLPVPQPEIMEVPK

SEQ ID NO: 219 - protein, 19 aa, Homo sapiens
QPAVVLPVPQPEIMEVPKA

SEQ ID NO: 220 - protein, 20 aa, Homo sapiens
QPAVVLPVPQPEIMEVPKAK

SEQ ID NO: 221 - protein, 25 aa, Homo sapiens
QPAVVLPVPQPEIMEVPKAKDTVYT

SEQ ID NO: 222 - protein, 26 aa, Homo sapiens
QPAVVLPVPQPEIMEVPKAKDTVYTK

SEQ ID NO: 223 - protein, 7 aa, Homo sapiens
QPLAPVH

SEQ ID NO: 224 - protein, 12 aa, Homo sapiens
QPLAPVHNPISV

SEQ ID NO: 225 - protein, 8 aa, Homo sapiens
QQVPQPIP

SEQ ID NO: 226 - protein, 8 aa, Homo sapiens
QVPQPIPQ

SEQ ID NO: 227 - protein, 10 aa, Homo sapiens
QVPQPIPQTL

SEQ ID NO: 228 - protein, 7 aa, Homo sapiens
RETIESL

SEQ ID NO: 229 - protein, 9 aa, Homo sapiens
RETIESLSS

SEQ ID NO: 230 - protein, 12 aa, Homo sapiens
RETIESLSSSEE

SEQ ID NO: 231 - protein, 14 aa, Homo sapiens
RETIESLSSSEESI

SEQ ID NO: 232 - protein, 16 aa, Homo sapiens
RETIESLSSSEESITE

SEQ ID NO: 233 - protein, 17 aa, Homo sapiens
RETIESLSSSEESITEY

SEQ ID NO: 234 - protein, 18 aa, Homo sapiens
RETIESLSSSEESITEYK

SEQ ID NO: 235 - protein, 19 aa, Homo sapiens
RETIESLSSSEESITEYKQ

SEQ ID NO: 236 - protein, 20 aa, Homo sapiens
RETIESLSSSEESITEYKQK

SEQ ID NO: 237 - protein, 22 aa, Homo sapiens
RETIESLSSSEESITEYKQKVE

SEQ ID NO: 238 - protein, 23 aa, Homo sapiens
RETIESLSSSEESITEYKQKVEK

SEQ ID NO: 239 - protein, 24 aa, Homo sapiens
RETIESLSSSEESITEYKQKVEKV

SEQ ID NO: 240 - protein, 25 aa, Homo sapiens
RETIESLSSSEESITEYKQKVEKVK

SEQ ID NO: 241 - protein, 27 aa, Homo sapiens
RETIESLSSSEESITEYKQKVEKVKHE

SEQ ID NO: 242 - protein, 32 aa, Homo sapiens
RETIESLSSSEESITEYKQKVEKVKHEDQQQG

SEQ ID NO: 243 - protein, 7 aa, Homo sapiens
SEESITE

SEQ ID NO: 244 - protein, 9 aa, Homo sapiens
SEESITEYK

SEQ ID NO: 245 - protein, 13 aa, Homo sapiens
SEESITEYKQKVE

SEQ ID NO: 246 - protein, 11 aa, Homo sapiens
SLSSSEESITE

SEQ ID NO: 247 - protein, 13 aa, Homo sapiens
SLSSSEESITEYK

SEQ ID NO: 248 - protein, 18 aa, Homo sapiens
SLSSSEESITEYKQKVEK

SEQ ID NO: 249 - protein, 7 aa, Homo sapiens
SPTIPFF

SEQ ID NO: 250 - protein, 8 aa, Homo sapiens
SPTIPFFD

SEQ ID NO: 251 - protein, 13 aa, Homo sapiens
SPTIPFFDPQIPK

SEQ ID NO: 252 - protein, 14 aa, Homo sapiens
SPTIPFFDPQIPKL

SEQ ID NO: 253 - protein, 16 aa, Homo sapiens
SPTIPFFDPQIPKLTD

SEQ ID NO: 254 - protein, 8 aa, Homo sapiens
SSEESITE

SEQ ID NO: 255 - protein, 9 aa, Homo sapiens
SSEESITEY

SEQ ID NO: 256 - protein, 10 aa, Homo sapiens
SSEESITEYK

SEQ ID NO: 257 - protein, 9 aa, Homo sapiens
SSSEESITE

SEQ ID NO: 258 - protein, 11 aa, Homo sapiens
SSSEESITEYK

SEQ ID NO: 259 - protein, 15 aa, Homo sapiens
SSSEESITEYKQKVE

SEQ ID NO: 260 - protein, 16 aa, Homo sapiens
SSSEESITEYKQKVEK

SEQ ID NO: 261 - protein, 20 aa, Homo sapiens
SVPQPKVLPIPQQVVPYPQR

SEQ ID NO: 262 - protein, 25 aa, Homo sapiens
SVPQPKVLPIPQQVVPYPQRAVPVQ

SEQ ID NO: 263 - protein, 26 aa, Homo sapiens
SVPQPKVLPIPQQVVPYPQRAVPVQA

SEQ ID NO: 264 - protein, 7 aa, Homo sapiens
TDLENLH

SEQ ID NO: 265 - protein, 9 aa, Homo sapiens
TDLENLHLP

SEQ ID NO: 266 - protein, 11 aa, Homo sapiens
TDLENLHLPLP

SEQ ID NO: 267 - protein, 8 aa, Homo sapiens
TEYKQKVE

SEQ ID NO: 268 - protein, 14 aa, Homo sapiens
TEYKQKVEKVKHED

SEQ ID NO: 269 - protein, 20 aa, Homo sapiens
THQIYPVTQPLAPVHNPISV

SEQ ID NO: 270 - protein, 14 aa, Homo sapiens
TIESLSSSEESITE

SEQ ID NO: 271 - protein, 15 aa, Homo sapiens
TIESLSSSEESITEY

SEQ ID NO: 272 - protein, 16 aa, Homo sapiens
TIESLSSSEESITEYK

SEQ ID NO: 273 - protein, 21 aa, Homo sapiens
TIESLSSSEESITEYKQKVEK

SEQ ID NO: 274 - protein, 8 aa, Homo sapiens
TQPLAPVH

SEQ ID NO: 275 - protein, 13 aa, Homo sapiens
TQPLAPVHNPISV

SEQ ID NO: 276 - protein, 19 aa, Homo sapiens
VEKVKHEDQQQGEDEHQDK

SEQ ID NO: 277 - protein, 23 aa, Homo sapiens
VEKVKHEDQQQGEDEHQDKIYPS

SEQ ID NO: 278 - protein, 11 aa, Homo sapiens
VEPIPYGFLPQ

SEQ ID NO: 279 - protein, 14 aa, Homo sapiens
VKHEDQQQGEDEHQ

SEQ ID NO: 280 - protein, 15 aa, Homo sapiens
VKHEDQQQGEDEHQD

SEQ ID NO: 281 - protein, 16 aa, Homo sapiens
VKHEDQQQGEDEHQDK

SEQ ID NO: 282 - protein, 19 aa, Homo sapiens
VKHEDQQQGEDEHQDKIYP

SEQ ID NO: 283 - protein, 20 aa, Homo sapiens
VKHEDQQQGEDEHQDKIYPS

SEQ ID NO: 284 - protein, 6 aa, Homo sapiens
VLPIPQ

SEQ ID NO: 285 - protein, 8 aa, Homo sapiens
VLPIPQQV

SEQ ID NO: 286 - protein, 10 aa, Homo sapiens
VLPIPQQVVP

SEQ ID NO: 287 - protein, 12 aa, Homo sapiens
VLPIPQQVVPYP

SEQ ID NO: 288 - protein, 13 aa, Homo sapiens
VLPIPQQVVPYPQ

SEQ ID NO: 289 - protein, 14 aa, Homo sapiens
VLPIPQQVVPYPQR

SEQ ID NO: 290 - protein, 15 aa, Homo sapiens
VLPIPQQVVPYPQRA

SEQ ID NO: 291 - protein, 19 aa, Homo sapiens
VLPIPQQVVPYPQRAVPVQ

SEQ ID NO: 292 - protein, 20 aa, Homo sapiens
VLPIPQQVVPYPQRAVPVQA

SEQ ID NO: 293 - protein, 21 aa, Homo sapiens
VLPIPQQVVPYPQRAVPVQAL

SEQ ID NO: 294 - protein, 9 aa, Homo sapiens
VLPVPQPEI

SEQ ID NO: 295 - protein, 10 aa, Homo sapiens
VLPVPQPEIM

SEQ ID NO: 296 - protein, 11 aa, Homo sapiens
VLPVPQPEIME

SEQ ID NO: 297 - protein, 14 aa, Homo sapiens
VLPVPQPEIMEVPK

SEQ ID NO: 298 - protein, 11 aa, Homo sapiens
VMPVLKSPTIP

SEQ ID NO: 299 - protein, 10 aa, Homo sapiens
VPKAKDTVYT

SEQ ID NO: 300 - protein, 12 aa, Homo sapiens
VPKAKDTVYTKG

SEQ ID NO: 301 - protein, 6 aa, Homo sapiens
VPQPIP

SEQ ID NO: 302 - protein, 7 aa, Homo sapiens
VPQPIPQ

SEQ ID NO: 303 - protein, 13 aa, Homo sapiens
VPQPKVLPIPQQV

SEQ ID NO: 304 - protein, 12 aa, Homo sapiens
VPYPQRAVPVQA

SEQ ID NO: 305 - protein, 14 aa, Homo sapiens
VTQPLAPVHNPISV

SEQ ID NO: 306 - protein, 12 aa, Homo sapiens
VVLPVPQPEIME

SEQ ID NO: 307 - protein, 15 aa, Homo sapiens
VVLPVPQPEIMEVPK

SEQ ID NO: 308 - protein, 16 aa, Homo sapiens
VVLPVPQPEIMEVPKA

SEQ ID NO: 309 - protein, 17 aa, Homo sapiens
VVLPVPQPEIMEVPKAK

SEQ ID NO: 310 - protein, 19 aa, Homo sapiens
VVLPVPQPEIMEVPKAKDT

SEQ ID NO: 311 - protein, 22 aa, Homo sapiens
VVLPVPQPEIMEVPKAKDTVYT

SEQ ID NO: 312 - protein, 23 aa, Homo sapiens
VVLPVPQPEIMEVPKAKDTVYTK

SEQ ID NO: 313 - protein, 24 aa, Homo sapiens
VVLPVPQPEIMEVPKAKDTVYTKG

SEQ ID NO: 314 - protein, 25 aa, Homo sapiens
VVLPVPQPEIMEVPKAKDTVYTKGR

SEQ ID NO: 315 - protein, 12 aa, Homo sapiens
VVPYPQRAVPVQ

SEQ ID NO: 316 - protein, 13 aa, Homo sapiens
VVPYPQRAVPVQA

SEQ ID NO: 317 - protein, 11 aa, Homo sapiens
YPVTQPLAPVH

SEQ ID NO: 318 - protein, 16 aa, Homo sapiens
YPVTQPLAPVHNPISV

SEQ ID NO: 319 - protein, 16 aa, Homo sapiens
QLAPIWDKLGETYKDH

SEQ ID NO: 320 - protein, 17 aa, Homo sapiens
ANPAVVRPHAQIPQRQY

SEQ ID NO: 321 - protein, 7 aa, Homo sapiens
HPPTVVR

SEQ ID NO: 322 - protein, 8 aa, Homo sapiens
LPNSHPPT

SEQ ID NO: 323 - protein, 9 aa, Homo sapiens
LPNSHPPTV

SEQ ID NO: 324 - protein, 11 aa, Homo sapiens
LPNSHPPTVVR

SEQ ID NO: 325 - protein, 9 aa, Homo sapiens
TTTVAVTPP

SEQ ID NO: 326 - protein, 13 aa, Homo sapiens
TYYANPAVVRPHA

SEQ ID NO: 327 - protein, 16 aa, Homo sapiens
TYYANPAVVRPHAQIP

SEQ ID NO: 328 - protein, 18 aa, Homo sapiens
TYYANPAVVRPHAQIPQR

SEQ ID NO: 329 - protein, 20 aa, Homo sapiens
TYYANPAVVRPHAQIPQRQY

SEQ ID NO: 330 - protein, 16 aa, Homo sapiens
YANPAVVRPHAQIPQR

SEQ ID NO: 331 - protein, 11 aa, Homo sapiens
IYDKTYAGGRL

SEQ ID NO: 332 - protein, 8 aa, Homo sapiens
DDEEKPKI

SEQ ID NO: 333 - protein, 6 aa, Homo sapiens
AGGGGL

SEQ ID NO: 334 - protein, 21 aa, Homo sapiens
DDPDAPLQPVTPLQLFEGRRN

SEQ ID NO: 335 - protein, 9 aa, Homo sapiens
ELTSWYFVS

SEQ ID NO: 336 - protein, 30 aa, Homo sapiens
KINVKVGGNSKGTLKVLRTYNVLDMKNTTC

SEQ ID NO: 337 - protein, 12 aa, Homo sapiens
AIPVAQDLNAPS

SEQ ID NO: 338 - protein, 13 aa, Homo sapiens
AIPVAQDLNAPSD

SEQ ID NO: 339 - protein, 9 aa, Homo sapiens
ATDEDITSH

SEQ ID NO: 340 - protein, 15 aa, Homo sapiens
DIQYPDATDEDITSH

SEQ ID NO: 341 - protein, 15 aa, Homo sapiens
DQSAETHSHKQSRLY

SEQ ID NO: 342 - protein, 8 aa, Homo sapiens
EDITSHME

SEQ ID NO: 343 - protein, 10 aa, Homo sapiens
ESEELNGAYK

SEQ ID NO: 344 - protein, 9 aa, Homo sapiens
GDSVVYGLR

SEQ ID NO: 345 - protein, 11 aa, Homo sapiens
HELDSASSEVN

SEQ ID NO: 346 - protein, 6 aa, Homo sapiens
IPVAQD

SEQ ID NO: 347 - protein, 11 aa, Homo sapiens
IPVAQDLNAPS

SEQ ID NO: 348 - protein, 8 aa, Homo sapiens
IPVKQADS

SEQ ID NO: 349 - protein, 9 aa, Homo sapiens
IPVKQADSG

SEQ ID NO: 350 - protein, 13 aa, Homo sapiens
ISHELDSASSEVN

SEQ ID NO: 351 - protein, 9 aa, Homo sapiens
NKYPDAVAT

SEQ ID NO: 352 - protein, 14 aa, Homo sapiens
RISHELDSASSEVN

SEQ ID NO: 353 - protein, 10 aa, Homo sapiens
RPDIQYPDAT

SEQ ID NO: 354 - protein, 11 aa, Homo sapiens
RPDIQYPDATD

SEQ ID NO: 355 - protein, 15 aa, Homo sapiens
RPDIQYPDATDEDIT

SEQ ID NO: 356 - protein, 17 aa, Homo sapiens
RPDIQYPDATDEDITSH

SEQ ID NO: 357 - protein, 28 aa, Homo sapiens
RPDIQYPDATDEDITSHMESEELNGAYK

SEQ ID NO: 358 - protein, 16 aa, Homo sapiens
RRPDIQYPDATDEDIT

SEQ ID NO: 359 - protein, 18 aa, Homo sapiens
RRPDIQYPDATDEDITSH

SEQ ID NO: 360 - protein, 29 aa, Homo sapiens
RRPDIQYPDATDEDITSHMESEELNGAYK

SEQ ID NO: 361 - protein, 9 aa, Homo sapiens
SEELNGAYK

SEQ ID NO: 362 - protein, 12 aa, Homo sapiens
SHELDSASSEVN

SEQ ID NO: 363 - protein, 18 aa, Homo sapiens
SKSKKFRRPDIQYPDATD

SEQ ID NO: 364 - protein, 24 aa, Homo sapiens
SKSKKFRRPDIQYPDATDEDITSH

SEQ ID NO: 365 - protein, 35 aa, Homo sapiens
SKSKKFRRPDIQYPDATDEDITSHMESEELNGAYK

SEQ ID NO: 366 - protein, 14 aa, Homo sapiens
TYDGRGDSVVYGLR

SEQ ID NO: 367 - protein, 12 aa, Homo sapiens
YPDATDEDITSH

SEQ ID NO: 368 - protein, 18 aa, Homo sapiens
DRSPYEKVSAGNGGSSLS

SEQ ID NO: 369 - protein, 14 aa, Homo sapiens
SPYEKVSAGNGGSS

SEQ ID NO: 370 - protein, 15 aa, Homo sapiens
SPYEKVSAGNGGSSL

SEQ ID NO: 371 - protein, 16 aa, Homo sapiens
SPYEKVSAGNGGSSLS

SEQ ID NO: 372 - protein, 21 aa, Homo sapiens
STDRSPYEKVSAGNGGSSLSY

SEQ ID NO: 373 - protein, 19 aa, Homo sapiens
TDRSPYEKVSAGNGGSSLS

SEQ ID NO: 374 - protein, 20 aa, Homo sapiens
TDRSPYEKVSAGNGGSSLSY

SEQ ID NO: 375 - protein, 32 aa, Homo sapiens
TDRSPYEKVSAGNGGSSLSYTNPAVAATSANL

SEQ ID NO: 376 - protein, 12 aa, Homo sapiens
TNPAVAATSANL

SEQ ID NO: 377 - protein, 26 aa, Homo sapiens
SGNHPITVHCSAGAGRTGTFCALSTV

SEQ ID NO: 378 - protein, 10 aa, Homo sapiens
EGGFVEGVNK

SEQ ID NO: 379 - protein, 17 aa, Homo sapiens
KLGAVYTEGGFVEGVNK

SEQ ID NO: 380 - protein, 31 aa, Homo sapiens
RQKASLTNVTDPSLDLTSLSLEVGCGAPAPV

SEQ ID NO: 381 - protein, 15 aa, Homo sapiens
DPSKPSSNVAGVVII

SEQ ID NO: 382 - protein, 16 aa, Homo sapiens
DPSKPSSNVAGVVIIV

SEQ ID NO: 383 - protein, 8 aa, Homo sapiens
QRHNSSIN

SEQ ID NO: 384 - protein, 19 aa, Homo sapiens
SLWNKDPLTSVSYQINSKS

SEQ ID NO: 385 - protein, 6 aa, Homo sapiens
AEMKLR

SEQ ID NO: 386 - protein, 9 aa, Homo sapiens
YRLNYSLPT

SEQ ID NO: 387 - protein, 11 aa, Homo sapiens
EKQTDEIKDTR

SEQ ID NO: 388 - protein, 14 aa, Homo sapiens
LQNPSESSEPIPLE

SEQ ID NO: 389 - protein, 16 aa, Homo sapiens
LQNPSESSEPIPLESR

SEQ ID NO: 390 - protein, 24 aa, Homo sapiens
LQNPSESSEPIPLESREEYMNGMN

SEQ ID NO: 391 - protein, 9 aa, Homo sapiens
MNRQRNILR

SEQ ID NO: 392 - protein, 10 aa, Homo sapiens
NILREKQTDE

SEQ ID NO: 393 - protein, 15 aa, Homo sapiens
NILREKQTDEIKDTR

SEQ ID NO: 394 - protein, 10 aa, Homo sapiens
NPSESSEPIP

SEQ ID NO: 395 - protein, 14 aa, Homo sapiens
NPSESSEPIPLESR

SEQ ID NO: 396 - protein, 22 aa, Homo sapiens
NPSESSEPIPLESREEYMNGMN

SEQ ID NO: 397 - protein, 9 aa, Homo sapiens
NYEKNNVML

SEQ ID NO: 398 - protein, 17 aa, Homo sapiens
QRNILREKQTDEIKDTR

SEQ ID NO: 399 - protein, 7 aa, Homo sapiens
RLQNPSE

SEQ ID NO: 400 - protein, 13 aa, Homo sapiens
RLQNPSESSEPIP

SEQ ID NO: 401 - protein, 15 aa, Homo sapiens
RLQNPSESSEPIPLE

SEQ ID NO: 402 - protein, 17 aa, Homo sapiens
RLQNPSESSEPIPLESR

SEQ ID NO: 403 - protein, 24 aa, Homo sapiens
RLQNPSESSEPIPLESREEYMNGM

SEQ ID NO: 404 - protein, 25 aa, Homo sapiens
RLQNPSESSEPIPLESREEYMNGMN

SEQ ID NO: 405 - protein, 26 aa, Homo sapiens
RLQNPSESSEPIPLESREEYMNGMNR

SEQ ID NO: 406 - protein, 7 aa, Homo sapiens
RPKLPLR

SEQ ID NO: 407 - protein, 10 aa, Homo sapiens
RPKLPLRYPE

SEQ ID NO: 408 - protein, 13 aa, Homo sapiens
RPKLPLRYPERLQ

SEQ ID NO: 409 - protein, 35 aa, Homo sapiens
RPKLPLRYPERLQNPSESSEPIPLESREEYMNGMN

SEQ ID NO: 410 - protein, 8 aa, Homo sapiens
YEKNNVML

SEQ ID NO: 411 - protein, 12 aa, Homo sapiens
AIYASKAVGEPP

SEQ ID NO: 412 - protein, 8 aa, Homo sapiens
DTSEAKKV

SEQ ID NO: 413 - protein, 8 aa, Homo sapiens
VPANRIVV

SEQ ID NO: 414 - protein, 21 aa, Homo sapiens
TRELDELMASLSDFKIQGLEQ

SEQ ID NO: 415 - protein, 31 aa, Homo sapiens
WPRDGGRSSPGGQDEGGFMAQGKTGSSSPPG

SEQ ID NO: 416 - protein, 12 aa, Homo sapiens
FPAPRGTPLISP

SEQ ID NO: 417 - protein, 9 aa, Homo sapiens
SGVVGLVNC

SEQ ID NO: 418 - protein, 14 aa, Homo sapiens
AESSKPTAGGSRSQ

SEQ ID NO: 419 - protein, 9 aa, Homo sapiens
LPPPPPPPP

SEQ ID NO: 420 - protein, 20 aa, Homo sapiens
GRGGRGGSRARNLPLPPPPP

SEQ ID NO: 421 - protein, 9 aa, Homo sapiens
LPLPPPPPP

SEQ ID NO: 422 - protein, 16 aa, Homo sapiens
TKIIEGGAAHKDGRLQ

SEQ ID NO: 423 - protein, 26 aa, Homo sapiens
ADINTPLTTSSGNLHGQPVSFLLREL

SEQ ID NO: 424 - protein, 25 aa, Homo sapiens
HHAAIEILQNAPEDVTLVISQPKEK

SEQ ID NO: 425 - protein, 12 aa, Homo sapiens
VPLPPARPPTRD

SEQ ID NO: 426 - protein, 18 aa, Homo sapiens
ADTLHSKLIPTQPSQGAP

SEQ ID NO: 427 - protein, 14 aa, Homo sapiens
APFDVIGPPEPILA

SEQ ID NO: 428 - protein, 22 aa, Homo sapiens
APRDADTLHSKLIPTQPSQGAP

SEQ ID NO: 429 - protein, 16 aa, Homo sapiens
DGPERVTVIANAQDLS

SEQ ID NO: 430 - protein, 13 aa, Homo sapiens
DGREQEAEQMPEY

SEQ ID NO: 431 - protein, 14 aa, Homo sapiens
DGREQEAEQMPEYR

SEQ ID NO: 432 - protein, 15 aa, Homo sapiens
DGREQEAEQMPEYRG

SEQ ID NO: 433 - protein, 16 aa, Homo sapiens
DGREQEAEQMPEYRGR

SEQ ID NO: 434 - protein, 6 aa, Homo sapiens
DVIGPP

SEQ ID NO: 435 - protein, 12 aa, Homo sapiens
EDSAPRDADTLH

SEQ ID NO: 436 - protein, 14 aa, Homo sapiens
EIPLSPMGEDSAPR

SEQ ID NO: 437 - protein, 20 aa, Homo sapiens
EIPLSPMGEDSAPRDADTLH

SEQ ID NO: 438 - protein, 12 aa, Homo sapiens
GRATLVQDGIAK

SEQ ID NO: 439 - protein, 16 aa, Homo sapiens
GRATLVQDGIAKGRVA

SEQ ID NO: 440 - protein, 13 aa, Homo sapiens
GREQEAEQMPEYR

SEQ ID NO: 441 - protein, 15 aa, Homo sapiens
GREQEAEQMPEYRGR

SEQ ID NO: 442 - protein, 10 aa, Homo sapiens
IPLSPMGEDS

SEQ ID NO: 443 - protein, 13 aa, Homo sapiens
IPLSPMGEDSAPR

SEQ ID NO: 444 - protein, 19 aa, Homo sapiens
IPLSPMGEDSAPRDADTLH

SEQ ID NO: 445 - protein, 11 aa, Homo sapiens
KEIPLSPMGED

SEQ ID NO: 446 - protein, 15 aa, Homo sapiens
KEIPLSPMGEDSAPR

SEQ ID NO: 447 - protein, 19 aa, Homo sapiens
KEIPLSPMGEDSAPRDADT

SEQ ID NO: 448 - protein, 21 aa, Homo sapiens
KEIPLSPMGEDSAPRDADTLH

SEQ ID NO: 449 - protein, 22 aa, Homo sapiens
KEIPLSPMGEDSAPRDADTLHS

SEQ ID NO: 450 - protein, 23 aa, Homo sapiens
KEIPLSPMGEDSAPRDADTLHSK

SEQ ID NO: 451 - protein, 31 aa, Homo sapiens
KEIPLSPMGEDSAPRDADTLHSKLIPTQPSQ

SEQ ID NO: 452 - protein, 34 aa, Homo sapiens
KEIPLSPMGEDSAPRDADTLHSKLIPTQPSQGAP

SEQ ID NO: 453 - protein, 6 aa, Homo sapiens
LPLAGP

SEQ ID NO: 454 - protein, 25 aa, Homo sapiens
QDLSKEIPLSPMGEDSAPRDADTLH

SEQ ID NO: 455 - protein, 11 aa, Homo sapiens
SKLIPTQPSQG

SEQ ID NO: 456 - protein, 13 aa, Homo sapiens
SKLIPTQPSQGAP

SEQ ID NO: 457 - protein, 16 aa, Homo sapiens
SPMGEDSAPRDADTLH

SEQ ID NO: 458 - protein, 9 aa, Homo sapiens
TLVQDGIAK

SEQ ID NO: 459 - protein, 13 aa, Homo sapiens
TLVQDGIAKGRVA

SEQ ID NO: 460 - protein, 21 aa, Homo sapiens
SPLPHSSPPTAAVATTSITTA

SEQ ID NO: 461 - protein, 15 aa, Homo sapiens
LPSKTPPPPPPKTTR

SEQ ID NO: 462 - protein, 35 aa, Homo sapiens
AAFKTLSGAQDSEAAFAKLDQKDLVLPTQALPASP

SEQ ID NO: 463 - protein, 22 aa, Homo sapiens
PPPPPPPPLLPGSSAEPPPPPP

SEQ ID NO: 464 - protein, 8 aa, Homo sapiens
SEGDGGRL

SEQ ID NO: 465 - protein, 20 aa, Homo sapiens
LEKQLESSQARKAMEEFFSD

SEQ ID NO: 466 - protein, 10 aa, Homo sapiens
EKLSALKISN

SEQ ID NO: 467 - protein, 18 aa, Homo sapiens
KVNMISREQFDTLTPEPP

SEQ ID NO: 468 - protein, 27 aa, Homo sapiens
AAEPPTLISPQAPASSPSSLSTSPPEV

SEQ ID NO: 469 - protein, 8 aa, Homo sapiens
RPPPPPPP

SEQ ID NO: 470 - protein, 14 aa, Homo sapiens
TKVGEIFSAAGAAF

SEQ ID NO: 471 - protein, 51 aa, Homo sapiens
LNLSENPAMTEGLLRAQVDSSFLSLYDSHVAKEILLRVLTLFQNIKNCLKI

SEQ ID NO: 472 - protein, 20 aa, Homo sapiens
QRTSSIATALNTSGAGGSRP

SEQ ID NO: 473 - protein, 13 aa, Homo sapiens
SLDDIDLSALRDP

SEQ ID NO: 474 - protein, 14 aa, Homo sapiens
SVALPPPPGPPPPP

SEQ ID NO: 475 - protein, 9 aa, Homo sapiens
PPAPPPPPP

SEQ ID NO: 476 - protein, 9 aa, Homo sapiens
PPPAPPPPP

SEQ ID NO: 477 - protein, 13 aa, Homo sapiens
DLLVEILMRPTIS

SEQ ID NO: 478 - protein, 6 aa, Homo sapiens
ERPPPP

SEQ ID NO: 479 - protein, 18 aa, Homo sapiens
NRNDQEATLEMLFPSRTT

SEQ ID NO: 480 - protein, 8 aa, Homo sapiens
IIGGFWLG

SEQ ID NO: 481 - protein, 34 aa, Homo sapiens
PMRRKSGPSCKHCKDDVNRLCRVCACHLCGGRQD

SEQ ID NO: 482 - protein, 9 aa, Homo sapiens
TTLIQYTSN

SEQ ID NO: 483 - protein, 18 aa, Homo sapiens
AEMDKSSQETQRSEHKTH

SEQ ID NO: 484 - protein, 21 aa, Homo sapiens
DQGAEMDKSSQETQRSEHKTH

SEQ ID NO: 485 - protein, 17 aa, Homo sapiens
EMDKSSQETQRSEHKTH

SEQ ID NO: 486 - protein, 10 aa, Homo sapiens
LPIIQKLEPQ

SEQ ID NO: 487 - protein, 12 aa, Homo sapiens
LPIIQKLEPQIA

SEQ ID NO: 488 - protein, 11 aa, Homo sapiens
LVSSGVENALT

SEQ ID NO: 489 - protein, 9 aa, Homo sapiens
VMDKTKGAV

SEQ ID NO: 490 - protein, 17 aa, Homo sapiens
KGYPRNISHNWMHCRPR

SEQ ID NO: 491 - protein, 13 aa, Homo sapiens
WGRGNFTEGKVPH

SEQ ID NO: 492 - protein, 19 aa, Homo sapiens
CAQRDNPRASSPSRATRDN

SEQ ID NO: 493 - protein, 11 aa, Homo sapiens
PRPLHPPPPPP

SEQ ID NO: 494 - protein, 9 aa, Homo sapiens
DSSVASQIT

SEQ ID NO: 495 - protein, 34 aa, Homo sapiens
LAKGNAGKVNLPKELPADAVNLTIPASLDLSPLL

SEQ ID NO: 496 - protein, 13 aa, Homo sapiens
LGEKLGGNVVVSL

SEQ ID NO: 497 - protein, 23 aa, Homo sapiens
TVLGNGSSLSLPEGQSLRLVCAV

SEQ ID NO: 498 - protein, 27 aa, Homo sapiens
TVLGNGSSLSLPEGQSLRLVCAVDAVD

SEQ ID NO: 499 - protein, 29 aa, Homo sapiens
PSAPTAHPQPRPPQGPLALPGPSYAGNSP

SEQ ID NO: 500 - protein, 14 aa, Homo sapiens
MIYTYSGLFCVTVN

SEQ ID NO: 501 - protein, 16 aa, Homo sapiens
DHLVCFLPGTLALGVY

SEQ ID NO: 502 - protein, 10 aa, Homo sapiens
QLVSLLHMSL

SEQ ID NO: 503 - protein, 9 aa, Homo sapiens
SPPPPPPPP

SEQ ID NO: 504 - protein, 55 aa, Homo sapiens
YQRRPAIAINNPYVPRTYYANPAVVRPHAQIPQRQYLPNSHPPTVVRRPNLHPSF

SEQ ID NO: 505 - protein, 13 aa, Homo sapiens
QPTIPFFDPQIPK

SEQ ID NO: 506 - protein, 26 aa, Homo sapiens
QELLLNPTHQYPVTQPLAPVHNPISV

SEQ ID NO: 507 - protein, 15 aa, Homo sapiens
DQQQGEDEHQDKIYP

SEQ ID NO: 508 - protein, 11 aa, Homo sapiens
EESITEYKQKV

SEQ ID NO: 509 - protein, 13 aa, Homo sapiens
EVPKAKDTVYTKG

SEQ ID NO: 510 - protein, 21 aa, Homo sapiens
AQPAVVLPVPQPEIMEVPKAK

SEQ ID NO: 511 - protein, 14 aa, Homo sapiens
LPVPQPEIMEVPKA

SEQ ID NO: 512 - protein, 23 aa, Homo sapiens
DQADGSRASVDSGSSEEQGGSSR

SEQ ID NO: 513 - protein, 14 aa, Homo sapiens
GSSEEQGGSSRALV

SEQ ID NO: 514 - protein, 10 aa, Homo sapiens
PDPAKQTDRV

SEQ ID NO: 515 - protein, 12 aa, Homo sapiens
VTAEKAPPPPPP

SEQ ID NO: 516 - protein, 20 aa, Homo sapiens
ATDEDITSHMESEELNGAYK

SEQ ID NO: 517 - protein, 17 aa, Homo sapiens
EDITSHMESEELNGAYK

SEQ ID NO: 518 - protein, 26 aa, Homo sapiens
DIQYPDATDEDITSHMESEELNGAYK

SEQ ID NO: 519 - protein, 16 aa, Homo sapiens
DDQSAETHSHKQSRLY

SEQ ID NO: 520 - protein, 25 aa, Homo sapiens
AKSQTEQTQPLSLSLKPDPLAHLSM

SEQ ID NO: 521 - protein, 17 aa, Homo sapiens
SFRVRASSDGEGTMSRP

SEQ ID NO: 522 - protein, 11 aa, Homo sapiens
CSSPNDSEHGP

SEQ ID NO: 523 - protein, 10 aa, Homo sapiens
QWLHTQVGVH

SEQ ID NO: 524 - protein, 33 aa, Homo sapiens
LAGDALLSLLAGDLGVEVPSAVPRPTLEPAEQL

SEQ ID NO: 525 - protein, 10 aa, Homo sapiens
EHSESTLNVM

SEQ ID NO: 526 - protein, 30 aa, Homo sapiens
GLNYHKRCAFSIPNNCSGARKRRLSSTSLA

SEQ ID NO: 527 - protein, 19 aa, Homo sapiens
AVSEHQLLHDKGKSIQDLR

SEQ ID NO: 528 - protein, 20 aa, Homo sapiens
IIIGIGNSGGDLAVEISQTA

SEQ ID NO: 529 - protein, 6 aa, Homo sapiens
THTVTY

SEQ ID NO: 530 - protein, 13 aa, Homo sapiens
GPEAAKSDETAAK

SEQ ID NO: 531 - protein, 28 aa, Homo sapiens
GGGGGGGGGGGGGGGGEAGAVAPYGYTR

SEQ ID NO: 532 - protein, 9 aa, Homo sapiens
SPPPPPPPP

SEQ ID NO: 533 - protein, 10 aa, Homo sapiens
PPPLPPPPPP

SEQ ID NO: 534 - protein, 7 aa, Homo sapiens
IPPPPPP

SEQ ID NO: 535 - protein, 10 aa, Homo sapiens
YPPPPPPPPP

SEQ ID NO: 536 - protein, 6 aa, Homo sapiens
TDLENL

SEQ ID NO: 537 - protein, 7 aa, Homo sapiens
PTHQIYP

SEQ ID NO: 538 - protein, 22 aa, Homo sapiens
DTVYTKGRVMPVLKGRVMPVLK

SEQ ID NO: 539 - protein, 13 aa, Homo sapiens
NPSESSEPIPLES

SEQ ID NO: 540 - protein, 6 aa, Homo sapiens
EDITSH

SEQ ID NO: 541 - protein, 7 aa, Homo sapiens
EDITSHM

SEQ ID NO: 542 - protein, 17 aa, Homo sapiens
RETIESLSSEESITEYK

SEQ ID NO: 543 - protein, 5 aa, Artificial Sequence
  Description of Artificial Sequence Synthetic peptide
GGGGS

SEQ ID NO: 544 - protein, 4 aa, Homo sapiens
RETI

SEQ ID NO: 545 - protein, 4 aa, Homo sapiens
TEYK

SEQ ID NO: 546 - protein, 5 aa, Homo sapiens
RETIE

SEQ ID NO: 547 - protein, 5 aa, Homo sapiens
ITEYK

SEQ ID NO: 548 - protein, 6 aa, Homo sapiens
RETIES

SEQ ID NO: 549 - protein, 6 aa, Homo sapiens
SITEYK

SEQ ID NO: 550 - protein, 8 aa, Homo sapiens
RETIESLS

SEQ ID NO: 551 - protein, 46 aa, Homo sapiens
YGETAAVYVAVEERKAAGSRDVSLAKADAAPDEKVLDSGFREIENK

SEQ ID NO: 552 - protein, 51 aa, Homo sapiens
AIQDPRLFAEEKAVADTRDQADGSRASVDSGSSEEQGGSSRALVSTLVPLG

SEQ ID NO: 553 - protein, 70 aa, Homo sapiens
MKVLILACLVALALARETIESLSSSEESITEYKQKVEKVKHEDQQQGEDEHQDKIYPSFQPQPLIYPFVE

SEQ ID NO: 554 - protein, 52 aa, Homo sapiens
VEPIPYGFLPQNILPLAQPAVVLPVPQPEIMEVPKAKDTVYTKGRVMPVLKS

SEQ ID NO: 555 - protein, 56 aa, Homo sapiens
DTVYTKGRVMPVLKSPTIPFFDPQIPKLTDLENLHLPLPLLQPLMQQVPQPIPQTL

SEQ ID NO: 556 - protein, 30 aa, Homo sapiens
PLWSVPQPKVLPIPQQVVPYPQRAVPVQAL

SEQ ID NO: 557 - protein, 41 aa, Homo sapiens
MAVFPSSGLPRCLLTLILLQLPKLDSAPFDVIGPPEPILAV

SEQ ID NO: 558 - protein, 41 aa, Homo sapiens
LVHRDGREQEAEQMPEYRGRATLVQDGIAKGRVALRIRGVR

SEQ ID NO: 559 - protein, 8 aa, Homo sapiens
LPLAGPPR

SEQ ID NO: 560 - protein, 52 aa, Homo sapiens
IADGPERVTVIANAQDLSKEIPLSPMGEDSAPRDADTLHSKLIPTQPSQGAP

SEQ ID NO: 561 - protein, 54 aa, Homo sapiens
ARPKLPLRYPERLQNPSESSEPIPLESREEYMNGMNRQRNILREKQTDEIKDTR

SEQ ID NO: 562 - protein, 13 aa, Homo sapiens
HENYEKNNVMLQW

SEQ ID NO: 563 - protein, 44 aa, Homo sapiens
MRIAVICFCLLGITCAIPVKQADSGSSEEKQLYNKYPDAVATWL

SEQ ID NO: 564 - protein, 51 aa, Homo sapiens
VDTYDGRGDSVVYGLRSKSKKFRRPDIQYPDATDEDITSHMESEELNGAYK

SEQ ID NO: 565 - protein, 43 aa, Homo sapiens
AIPVAQDLNAPSDWDSRGKDSYETSQLDDQSAETHSHKQSRLY

SEQ ID NO: 566 - protein, 15 aa, Homo sapiens
ALPIIQKLEPQIAVA

SEQ ID NO: 567 - protein, 48 aa, Homo sapiens
TGVMDKTKGAVTGSVEKTKSVVSGSINTVLGSRMMQLVSSGVENALTK

SEQ ID NO: 568 - protein, 23 aa, Homo sapiens
AQDQGAEMDKSSQETQRSEHKTH

SEQ ID NO: 569 - protein, 36 aa, Homo sapiens
PPSSTDRSPYEKVSAGNGGSSLSYTNPAVAATSANL

SEQ ID NO: 570 - protein, 33 aa, Homo sapiens
RTYYANPAVVRPHAQIPQRQYLPNSHPPTVVRR

SEQ ID NO: 571 - protein, 28 aa, Homo sapiens
VVTPEAFSESIITSTPETTTVAVTPPTA

SEQ ID NO: 572 - protein, 9 aa, Homo sapiens
DSGSSEEQG